(19) United States
(12) Patent Application Publication
(10) Pub. No.: US 2021/0147544 A1
(43) Pub. Date: May 20, 2021

(54) METHODS AND COMPOSITIONS FOR TARGETED DELIVERY OF ACTIVE AGENTS AND IMMUNOMODULATORY AGENTS TO LYMPH NODES

(71) Applicant: IMMUNOVACCINE TECHNOLOGIES INC., Dartmouth (CA)

(72) Inventors: Rajkannan RAJAGOPALAN, Dartmouth (CA); Leeladhar SAMMATUR, Irvine, CA (US); Marianne STANFORD, Upper Tantallon (CA); Heather TORREY, Halifax (CA); Genevieve WEIR, Lawrencetown (CA)

(73) Assignee: IMMUNOVACCINE TECHNOLOGIES INC., Dartmouth, NS (CA)

(21) Appl. No.: 16/981,758

(22) PCT Filed: Mar. 18, 2019
(86) PCT No.: PCT/CA2019/050328

§ 371 Date: Sep. 17, 2020

Related U.S. Application Data
(60) Provisional application No. 62/645,249, filed on Mar. 20, 2018.

(51) Int. Cl. C07K 16/28; A61K 45/06

(52) U.S. Cl. CPC C07K 16/2818; A61K 2039/505; A61K 45/06

(57) ABSTRACT

The present disclosure relates to the targeted delivery of defined active agents and/or immunomodulatory agents to lymph nodes or lipoid cells in a lymphatic tissue. More particularly, the invention provides methods for targeted delivery of a defined active agent or immunomodulatory agent to lymph nodes or lymphoid cells in a lymphatic tissue, comprising administering to a subject in need thereof a composition comprising the active agent or immunomodulatory agent, one or more lipid-based structures, and a hydrophobic carrier.

### FIELD

This application claims the benefit of and priority from U.S. Provisional Patent Application No. 62/645,249 filed Mar. 20, 2018, incorporated by reference herein in its entirety.

The present application relates to methods and compositions for targeted delivery of active agents (e.g. small molecule drugs and antibodies) and immunomodulatory agents, to lymph nodes or lymphoid cells in a lymphatic tissue.

### BACKGROUND

Drugs, small molecules and antibodies are actively under investigation as potential therapies for many different types of diseases and disorders, including cancer and infectious disease. Most pharmaceuticals are small molecules—they are potent, structurally defined and are generally cost effective.

Generally, small molecules, drugs and antibodies are administered as oral tablets or intravenous (IV) injection. These administration routes result in systemic delivery of the small molecule, drug or antibody. Many immunomodulatory agents are active primarily on specific immune cells, such as dendritic cells or T cells, and administering them systemically may result in lower efficacy and potential off-target toxicity.

Various approaches of targeted therapies have been developed over the years. One approach has been to couple active agents to a targeting agent, such as an antibody. The antibody is used to change the biodistribution of the active agent such that more of it can localize where it is needed in vivo. As an alternative to antibodies, liposomal nanoparticles conjugated to targeting ligands have gained attention for their potential to provide selective delivery of therapeutic agents with reduced side effects. The challenge has been to identify a ligand that provides sufficient selectivity for the targeted cell type. Immuno-liposomes use antibodies as targeting agents, but have not to date provided a therapeutic index commensurate with their promise.

Because lymph nodes are the primary site for the priming and activation of immune cells (e.g. cytotoxic CD8+ T cells) in a wide variety of diseases and disorders, it is essential to develop effective methods for targeted delivery of drugs to lymph nodes. Targeted delivery of drugs to lymph nodes and lymphoid cells in lymphatic tissues holds the potential to increase the efficacy of immune and cancer therapies. Targeted delivery may also allow for reduced dosing as compared to systemic delivery, with corresponding reductions in off-target toxicities.

There is therefore a need in the art for new and effective means for targeted delivery of active agents and immunomodulatory agents to lymph nodes or lymphoid cells in a lymphatic tissue with the aim of increasing efficacy and potency, and reducing off-target side effects.

### SUMMARY

In an embodiment, the present disclosure relates to a method for targeted delivery of an active agent to lymph nodes or lymphoid cells in a lymphatic tissue, said method comprising administering to a subject in need thereof a composition comprising: (a) an active agent, wherein the active agent is a small molecule drug; an antibody, an antibody mimetic, or a functional equivalent or functional fragment of any one thereof; or a mixture thereof, (b) one or more lipid-based structures, and (c) a hydrophobic carrier.

In an embodiment, the present disclosure relates to a method for targeted delivery of an immunomodulatory agent to lymph nodes or lymphoid cells in a lymphatic tissue, said method comprising administering to a subject in need thereof a composition comprising: (a) an immunomodulatory agent, (b) one or more lipid-based structures, and (c) a hydrophobic carrier.

In an embodiment, the methods disclosed herein are for modulating an immune response in a subject.

In an embodiment, the methods disclosed herein are for treating or preventing a disease or disorder in the subject.

In an embodiment, the present disclosure relates to the use of a composition comprising: (a) an active agent, wherein the active agent is a small molecule drug; an antibody, an antibody mimetic, or a functional equivalent or functional fragment of any one thereof; or a mixture thereof; (b) one or more lipid-based structures; and (c) a hydrophobic carrier, for targeting the active agent to lymph nodes or lymphoid cells in a lymphatic tissue in a subject.

In an embodiment, the present disclosure relates to the use of a composition comprising: (a) an immunomodulatory agent; (b) one or more lipid-based structures; and (c) a hydrophobic carrier, for targeting the immunomodulatory agent to lymph nodes or lymphoid cells in a lymphatic tissue in a subject.

In an embodiment, the uses disclosed herein are for modulating an immune response in a subject.

In an embodiment, the uses disclosed herein are for treating or preventing a disease or disorder in the subject.

Other aspects and features of the present invention will become apparent to those of ordinary skill in the art upon review of the following description in conjunction with the accompanying figures.

### BRIEF DESCRIPTION OF THE FIGURES

The accompanying figures, which constitute a part of this specification, illustrate embodiments of the invention by way of example only:

FIG. 1 shows photographs of a CPA Composition according to the invention (Panel A), an FP/DNA based PolyI:C Composition (Panel B), a CPA/FP/DNA based PolyI:C Composition according the invention (Panel C), a FP/EPA/ DNA based PolyI:C Composition (Panel D), and a FP/Anti-CTL-4/DNA based PolyI:C Composition (Panel E.

FIG. 2 shows an HPLC chromatogram of a reference standard containing 15 μg/mL CPA.

FIG. 3 shows an HPLC chromatogram of a sample of a CPA preparation after freeze-drying, obtained from the manufacture of a CPA Composition according to the invention.

FIG. 4 shows an HPLC chromatogram of a sample of a CPA preparation after freeze-drying, obtained from the manufacture of a CPA/FP/PolyI:C Composition according to the invention.

FIG. 5 shows the total cell counts in the inguinal lymph node draining the injection with the FP antigen. Groups A, B, E and F were injected in the left side with a composition containing FP antigen, and left lymph node cell counts are shown. Groups C and D were injected in the right side with a composition containing the FP antigen, and right lymph node cell counts are shown. Statistics performed by student's t-test (unpaired, one-tailed) comparing each group to group F, *p<0.05, **p<0.01.

FIG. 6 shows (A) Schedule of experimental treatment; (B) Tumour growth; (C) Survival; and (D) Body Weights of mice during experimental treatment. DPX is administered by subcutaneous injection. Oral administration denoted as PO. Statistics of survival by Mantel-Cox *p<0.0332 comparing, DPX-FP/EPA mCPA (PO) and DPX-FP mCPA (PO) EPA (PO). Statistics of body weights by two-way ANOVA with Turkey's multiple comparison test **p<0.01, ***p<0.0005 and tumour volumes by linear regression **p<0.005, ***p<0.0005.

FIG. 7 shows (A) Schedule of experimental treatment; (B) Survival of mice monitored from study day 0 to 72; and (C) Tumour volume (mm3) in mice monitored from study day 0 to 72. Survival statistical analysis was performed using the Mantel-Cox test, ***p<0.001, *p<0.05. Tumour volume statistical analysis was performed by linear regression comparison, ***p<0.0001.

FIG. 8 shows (A) % of CD3+ T cells in the blood that are IgG2b+ on study day 16 and 30 and (B) % of CD8+ T cells in the blood that are IgG2b+ on study day 16 and 30. Blood samples collected at study day 16 (n=4) and 30 (n=4, No treatment n=2) assessed for mouse IgG2b bound to T cells by flow cytometry. Statistics performed by two-way ANOVA with Turkey's multiple comparison test *p<0.05, ***p<0.001.

FIG. 9 shows (A) Detection of anti-drug antibody (ADA) against anti-CTLA-4 (anti-CTLA-4 coating and detection antibody); (B) Control ELISA using IgG2b isotype control coating antibody and anti-CTLA-4 detection antibody; and (C) Control ELISA using IgG1 isotype control coating antibody and anti-CTLA-4 detection antibody. Serum samples collected from mice at 28 and 42 days post-injection (one of the DPX-FP, mCPA (water) samples was collected on day 41) assessed for ADA formation by bridging ELISA. Significant difference was detected by two-way ANOVA using Tukey's multiple comparisons test, *p<0.05. Detection limit indicated by dashed line.

FIG. 10 shows average percent±SEM of CD11b⁺ macrophages (A, D, G, J), CD11c⁺ dendritic cells (B, E, H, K), and CD3⁺ T cells (C, F, I, L) that are positive for Evans Blue (EVB) dye over time in the vaccine-draining inguinal lymph nodes (LN; A, B, C), blood (D, E, F), liver (G, H, I), and spleen (J, K, L), measured by flow cytometry. *p<0.05, **p<0.01, ***p<0.001, ****p<0.0001, Two-way ANOVA, Tukey's Multiple Comparison Test compared to control group 3.

FIG. 11 shows average percent±SEM of CD11b⁺macrophages (A, D, G, J), CD11c⁺dendritic cells (B, E, H, K), and CD3⁺ T cells (C, F, I, L) that are positive for AlexaFluor488 (AF488) dye over time in the vaccine-draining inguinal lymph nodes (LN; A, B, C), blood (D, E, F), liver (G, H, I), and spleen (J, K, L), measured by flow cytometry. *p<0.05, **p<0.01, ***p<0.001, ****p<0.0001, Two-way ANOVA, Tukey's Multiple Comparison Test compared to control group 3.

FIG. 12 shows photographs of plasma taken from the blood of (A) group 1 mice injected with EVB in DPX and (B) group 2 mice injected with EVB in an aqueous solution at days 1, 2, 5, and 7 after injection.

FIG. 13 shows photographs of (A) skin of a mouse group 2 injected with EVB in an aqueous solution; (B) blue draining lymph node of a group 1 mouse injected with EVB in DPX.

### DETAILED DESCRIPTION

The present invention relates to methods and compositions for targeted delivery of an active agent and/or an immunomodulatory agent to lymph nodes or lymphoid cells in a lymphatic tissue.

As used herein, by “targeted” or “targeting”, it is meant that the active agent and/or immunomodulatory agent is preferentially delivered to lymph nodes or lymphoid cells in a lymphatic tissue.

In an embodiment, “preferentially delivered” refers to the fact that the active agent and/or immunomodulatory agent is delivered to the lymph nodes or lymphoid cells in a lymphatic tissue as opposed to being delivered to other regions of the body, being delivered systemically or being eliminated (e.g. excreted) from the body without effective delivery. In an embodiment, by “preferentially delivered”, it is meant that the concentration or amount of the active agent and/or immunomodulatory agent is increased in the lymph nodes or lymphoid cells in a lymphatic tissue relative to the concentration or amount of the active agent and/or immunomodulatory agent in other parts of the body. In an embodiment, by “preferentially delivered”, it is meant that the active agent and/or immunomodulatory agent is delivered and taken up by cells in the lymph nodes or lymphatic tissue more effectively than if the active agent and/or immunomodulatory agent were not delivered in a composition of the invention.

As used herein, and without being bound by theory, the term “targeted delivery” encompasses embodiments whereby the targeting to the lymph nodes or lymphoid cells in a lymphatic tissue is accomplished by upstream events whereby the active agents and/or immunomodulatory agents are more effectively delivered to cells that are capable of trafficking the active agent and/or immunomodulatory agent to lymph nodes or lymphatic tissue. In an embodiment, the cells may be immune cells, such as for example and without limitation, monocytes, macrophages, dendritic cells, T cells and/or B cells. Thus, in an embodiment, “targeted delivery to lymph nodes or lymphoid cells in a lymphatic tissue” includes preferential delivery of the active agent and/or immunomodulatory agent to immune cells in a non-lymphatic fluid or tissue in the body whereby cells are then trafficked to lymph nodes or lymphoid cells in a lymphatic tissue to provide targeted delivery.

As used herein, and without being bound by theory, the term “targeted delivery” encompasses embodiments whereby the targeting to the lymph nodes or lymphoid cells in a lymphatic tissue is accomplished by a more effective delivery (e.g. trafficking) and/or uptake of the active agent and/or immunomodulatory agent to cells in the lymph nodes or lymphatic tissue.

In an embodiment, “targeted delivery” means that by using a composition of the invention as disclosed herein, the active agent and/or immunomodulatory agent is delivered to lymph nodes or lymphoid cells in a lymphatic tissue more effectively than by using a comparable composition that does not comprise one or both of the lipid-based structures and the hydrophobic carrier.

In an embodiment, “targeted delivery” means that by using a composition of the invention as disclosed herein, the active agent and/or immunomodulatory agent is delivered to lymph nodes or lymphoid cells in a lymphatic tissue more effectively than by oral or intravenous administration of the active agent and/or immunomodulatory agent alone or in a different composition (i.e. not a composition of the invention). In an embodiment, the methods and compositions disclosed herein are thereby capable of achieving an equivalent or better therapeutic result in the lymph nodes while using less active agent and/or immunomodulatory agent as compared to oral or intravenous administration of the agent(s) alone or in a different composition.

As used herein, “lymph nodes” refers to any one or more lymph nodes that are present throughout the body of an animal, such as for example a human. In an embodiment, the lymph nodes are any one or more of the following types, based on anatomical location: inguinal (groin), femoral (upper inner thigh), mesentery (lower body below rib cage), mediastinal (upper body behind the sternum); supraclavicular (collar bone); axillary (armpits); and cervical (neck). The lymph node to which the methods and compositions preferentially target may depend on the route of administration (e.g. injection) and location of administration. In an embodiment, the lymph nodes are the inguinal lymph nodes.

As used herein, the term “lymphatic tissue” refers to the cells and organs that make up the lymphatic system. It includes, without limitation, the lymph nodes, spleen, thymus and mucosal-associated lymphoid tissue (e.g., in the lung, lamina propria of the of the intestinal wall, Peyer's patches of the small intestine, or lingual, palatine and pharyngeal tonsils, or Waldeyer's neck ring). The lymphoid cells of the lymphatic tissue include, for example, leukocytes (white blood cells), T cells (T-lymphocytes), B cells (B-lymphocytes), macrophages, dendritic cells and reticular cells. In an embodiment, the targeted delivery of the methods and compositions disclosed herein is to T-lymphocytes and/or B-lymphocytes in the lymph nodes or lymphatic tissue.

The methods and compositions of the present invention are advantageous for targeted delivery of the active agents and/or immunomodulatory agents as defined herein to lymph nodes or lymphoid cells in a lymphatic tissue. Without the need for complex steps of conjugating targeting moieties (such as ligands or antibodies) to active agents and/or immunomodulatory agents, it was found that active agents and/or immunomodulatory agents could be preferentially delivered to the lymph nodes using a composition comprising one or more lipid-based structures and a hydrophobic carrier.

As shown in Example 1 and FIG. 5, by administering a cytotoxic active agent in a composition comprising one or more lipid-based structures and a hydrophobic carrier, a significant reduction in the number of lymph node cells was observed as compared to a control (compare Groups B/C with Group F). Thus, the methods of the invention are effective in targeting the delivery of the active agent to the lymph nodes. This result was observed irrespective of whether the active agent was administered alone (Group B) or together with an antigen (Group C). Thus, targeted delivery of the active agent was independent of classical activation of immune cells (e.g. monocytes, macrophages, dendritic cells, T cells and/or B cells) by antigen to become antigen-presenting cells. This is further supported by the fact that in Group B, the active agent composition was administered in a different flank (left flank) than the vaccine composition (right flank).

Moreover, the methods of the invention provided surprisingly effective reductions in lymph node cells after only a single administration. With only a single administration of a composition of the invention comprising 0.4 mg of active agent, lymph node cell counts were reduced to a level equivalent to that achieved with daily oral administration for one week, totalling about 2.8 mg of active agent. Thus, an equivalent therapeutic benefit was achieved with about seven times less active agent by using the methods and compositions of the invention.

Methods

Methods are provided for targeted delivery of an active agent and/or immunomodulatory agent to lymph nodes or lymphoid cells in a lymphatic tissue.

In an embodiment, the method is for targeted delivery of an active agent to lymph nodes or lymphoid cells in a lymphatic tissue, comprising administering to a subject in need thereof a composition comprising: (a) an active agent, wherein the active agent is a small molecule drug; an antibody, an antibody mimetic, or a functional equivalent or functional fragment of any one thereof; or a mixture thereof, (b) one or more lipid-based structures, and (c) a hydrophobic carrier.

In an embodiment, the method is for targeted delivery of an immunomodulatory agent to lymph nodes or lymphoid cells in a lymphatic tissue, comprising administering to a subject in need thereof a composition comprising: (a) an immunomodulatory agent, (b) one or more lipid-based structures, and (c) a hydrophobic carrier.

The methods disclosed herein may find application in any instance in which it is desired to target the delivery of an active agent and/or immunomodulatory agent to lymph nodes or lymphoid cells in a lymphatic tissue of a subject. The subject may be a vertebrate, such as a fish, bird or mammal. In an embodiment, the subject is a mammal. In an embodiment, the subject is a human.

The compositions that may be used in the practice of the methods of the invention are described in greater detail elsewhere herein. In an embodiment, as described herein, the compositions are water-free and comprise a 100% oil-based (i.e. hydrophobic) carrier.

Without being bound by theory, it is believed that the compositions provide for targeted delivery of the active agents and immunomodulatory agents to lymph nodes or lymphoid cells in a lymphatic tissues by one or more of: (i) promoting effective uptake of the active agent and/or immunomodulatory agent by immune cells (e.g. monocytes, macrophages, dendritic cells, T cells and/or B cells) at or near the site of administration due to the formation of a unique ‘depot’ that attracts immune cells and provides extended exposure to the active agent and/or immunomodulatory agent; (ii) promoting migration of such immune cells to lymph nodes, even despite the absence of traditional processes of immune cell activation by presentable antigens (e.g. to become an activated antigen-presenting cell); and (iii) promoting uptake of the active agent and/or immunomodulatory agent by cells in the lymph nodes or lymphoid cells in a lymphatic tissue. Each of these elements not only represents a surprising advantage of the disclosed methods, but also a hurdle that was unexpectedly overcome.

Prior to encountering foreign antigen, immune cells (e.g. monocytes, macrophages, dendritic cells, T cells and/or B cells) exist in an immature state. Upon phagocytosis of a presentable antigen, these cells become activated resulting in an upregulated expression of MHC class I/II molecules and maturation into mature antigen-presenting cells that migrate to the lymph nodes where they interact with T cells and B cells by receptor-mediated interactions. In the case of immunotherapy, appropriate activation of immune cells typically also requires administration of an adjuvant to improve routing and adaptive immune responses. In the absence of these features (e.g. presentable antigen and/or a suitable adjuvant), it was not thought that effective targeted delivery to the lymph nodes would occur, in part because there would be: (1) a lack of activation, (2) a lack of maturation and active migration to lymph nodes, and/or (3) a lack of receptor-mediated interactions in lymph nodes or lymphatic tissue with T-lymphocytes and B-lymphocytes. However, as shown herein, the disclosed methods exhibit targeted delivery of active agent/immunomodulatory agent independent of classical activation of immune cells by antigen and adjuvant. That the active agent/immunomodulatory agent was taken up by cells in the lymph nodes is further evident by the cytotoxic reduction of lymph node cells.

Thus, in an embodiment, the methods disclosed herein provide for targeted delivery (and uptake) of the active agents and/or immunomodulatory agents defined herein to immune cells in the lymph nodes or lymphoid cells in a lymphatic tissue. The immune cells in the lymph nodes or lymphoid cells in a lymphatic tissue may include, without limitation, myeloid progenitor cells, monocytes, dendritic cells, macrophages, T-lymphocytes and/or B-lymphocytes. In an embodiment, the immune cells are T-lymphocytes or B-lymphocytes in the lymph nodes or lymphatic tissue. In an embodiment, the immune cells are T-lymphocytes or B-lymphocytes in the lymph nodes.

In an embodiment, administration of the composition is by injection. Injection may be, for example, by subcutaneous, subdermal, submucosal, intramuscular or intraperitoneal injection. In an embodiment, the administration is by way of subcutaneous injection. Administration is to a region of the body other than the lymph nodes. In an embodiment, the injection is into the subject's arm, leg, belly, or buttock, but any convenient site may be chosen for injection. In an embodiment, injection of the composition is to a region of the body that directly or indirectly drains to lymph nodes. In an embodiment, injection of the composition is into a tissue of the body. In an embodiment the tissue is epithelial tissue, connective tissue, muscle tissue or nervous tissue. In an embodiment, the tissue is epithelial tissue or muscle tissue.

Since the compositions of the invention comprise a hydrophobic carrier, injection of the compositions will form a ‘depot’ at the site of injection (i.e. the hydrophobic carrier being immiscible with the aqueous host environment). The one or more lipid-based structures stabilize the active agent and/or immunomodulatory agent in the hydrophobic carrier in this environment. It is understood that this combined effect will allow the components of the composition to continuously interact with the microenvironment for an extended period of time. In this regard, in an embodiment, the methods disclosed herein involve an active, rather than passive, uptake of active agents and/or immunomodulatory agents for targeted delivery to the lymph nodes or lymphatic tissue. In an embodiment, the active agent and/or immunomodulatory agent is delivered to immune cells (e.g. monocytes, macrophages, dendritic cells, T cells and/or B cells) at or near the site of administration of the composition. In an embodiment, the active agents and/or immunomodulatory agents are delivered to lymph nodes or lymphoid cells in a lymphatic tissue by immune cells. In an embodiment, the immune cells are dendritic cells or macrophages. In an embodiment, the immune cells are dendritic cells.

In an embodiment of the methods disclosed herein, at least 50%, at least 55%, at least 60%, at least 65%, at least 70%, at least 75%, at least 80%, at least 85%, at least 90%, at least at least 95%, at least 96%, at least 97%, at least 98% or at least 99% of the administered active agent and/or immunomodulatory agent is delivered to lymph nodes or to lymphoid cells in a lymphatic tissues. In an embodiment, at least 50%, at least 55%, at least 60%, at least 65%, at least 70%, at least 75%, at least 80%, at least 85%, at least 90%, at least at least 95%, at least 96%, at least 97%, at least 98% or at least 99% of the administered active agent and/or immunomodulatory agent is delivered to lymph nodes. In an embodiment, absent the use of compositions comprising either lipid-based structures or hydrophobic carrier, the active agent and/or immunomodulatory agent exhibits systemic delivery in a subject. In an embodiment, absent the use of compositions comprising either lipid-based structures or hydrophobic carrier, the active agent and/or immunomodulatory agent is cleared from the body of the subject without preferential delivery to the lymph nodes or lymphoid cells in a lymphatic tissue.

By targeted delivery of a substantial quantity of the active agents and immunomodulatory agents to the lymph nodes, an advantage may be provided in the reduction or avoidance of undesired immune responses and/or reactivity against certain active agents and/or immunomodulatory agents. Such undesired effects sometimes occur with systemic delivery of active agents and/or immunomodulatory agents, and are routinely mediated either by discontinuing administration of the active agent and/or immunomodulatory agent or by administering other drugs to block or reduce the undesired effects (e.g. immunosuppressants). These approaches are not desired since the subject would either not receive the required therapeutic treatment or treatment would involve additional costs and possibly undesired suppression of the immune system generally. This may be avoided by the methods of targeted delivery disclosed herein.

By targeted delivery of a substantial quantity of the active agents and immunomodulatory agents to the lymph nodes, a further advantage may be provided in the duration, ease, and acceptance of administration of the active agents and immunomodulatory agents to a patient. Some agents conventionally require intravenous administration, requiring that the patient be connected to a volumetric pump via a venous catheter for an extended period of time. By way of example, anti-CTLA-4 antibody is conventionally administered to patients 1-10 mg/kg intravenously over a period of 30-90 minutes. This may be avoided by the methods of targeted delivery disclosed herein.

In an embodiment, by the methods disclosed herein, targeted delivery of the active agent and/or immunomodulatory agent allows for reduced dosing as compared to alternative methods, such as systemic delivery by oral administration using a different composition (i.e. not a composition of the invention). In an embodiment, the total amount of active agent and/or immunomodulatory agent administered to a subject during a course of treatment using the methods of the invention may be only about 1%, 2%, 3%, 4%, 5%, 6%, 7%, 8%, 9%, 10%, 15%, 20%, 25%, 30%, 35%, 40%, 45%, 50%, 55%, 60%, 65%, 70% or 75% of the total amount of agent(s) needed using an alternative method. In an embodiment, the total amount of active agent and/or immunomodulatory agent administered to a subject during a course of treatment using the methods of the invention may be between 1-50%, 1-25%, 1-20%, 1-15%, 1-10% or 1-5% of the total amount of agent(s) needed using an alternative method. In an embodiment, the total amount of active agent and/or immunomodulatory agent administered to a subject during a course of treatment using the methods of the invention may be only about 2%, 3%, 4%, 5%, 6%, 7%, 8%, 9% or 10% of the total amount of agent(s) needed using an alternative method. By “course of treatment”, it is meant any length of time and/or any number of administrations to obtain the desired therapeutic, diagnostic or biological effect.

In an embodiment, by the methods disclosed herein, delivery of the active agent and/or immunomodulatory agent to lymph nodes or lymphoid cells in a lymphatic tissue does not activate an immune response and/or other undesired reactivity against the active agent and/or immunomodulatory agent. In a preferred embodiment of the methods herein, the active agent and/or immunomodulatory agent is a compound, substance or molecule that is non-immunogenic. In a further embodiment, it is contemplated that the active agents and/or immunomodulatory agents are delivered to the lymph nodes or lymphatic tissues within immune cells, possibly shielding any immunogenicity and/or reactivity that the active agent and/or immunomodulatory agent may display.

In an embodiment, the methods and uses disclosed herein are for modulating an immune response in a subject. By “modulate”, it is meant that the methods may be used to enhance (upregulate), suppress (downregulate), direct, redirect or reprogram an immune response in a subject. As used herein, “immune response” may be a cell-mediated immune response or an antibody (humoral) immune response.

Lymph nodes and lymphatic tissues are major sites of B-lymphocytes and T-lymphocytes, and are important sites in the body for the functioning of the immune system and development of immune responses. Methods that target the delivery of active agents and/or immunomodulatory agents to lymph nodes or lymphoid cells in a lymphatic tissue would be beneficial in modulating immune responses, whether it be a cell-mediated immune response, an antibody immune response, or both.

In an embodiment, the methods and uses disclosed herein are for modulating a cell-mediated immune response in a subject.

As used herein, the terms “cell-mediated immune response”, “cellular immunity”, “cellular immune response” or “cytotoxic T-lymphocyte (CTL) immune response” (used interchangeably herein) refer to an immune response characterized by the activation of macrophages and natural killer cells, the production of antigen-specific cytotoxic T lymphocytes and/or the release of various cytokines in response to an immunogen. Cytotoxic T lymphocytes are a sub-group of T lymphocytes (a type of white blood cell) which are capable of inducing the death of infected somatic or tumor cells; they kill cells that are infected with viruses (or other pathogens), or that are otherwise damaged or dysfunctional.

Cellular immunity protects the body by, for example, activating antigen-specific cytotoxic T-lymphocytes (e.g. antigen-specific CD8+ T cells) that are able to lyse body cells displaying epitopes of foreign or mutated antigen on their surface, such as cancer cells displaying tumor-specific antigens; activating macrophages and natural killer cells, enabling them to destroy intracellular pathogens; and stimulating cells to secrete a variety of cytokines that influence the function of other cells involved in adaptive immune responses and innate immune responses.

Cellular immunity is an important component of the adaptive immune response and following recognition of antigen by cells through their interaction with antigen-presenting cells such as dendritic cells, B lymphocytes and to a lesser extent, macrophages, protect the body by various mechanisms such as:

1. activating antigen-specific cytotoxic T-lymphocytes that are able to induce apoptosis in body cells displaying epitopes of foreign or mutated antigen on their surface, such as cancer cells displaying tumor-specific antigens;

2. activating macrophages and natural killer cells, enabling them to destroy intracellular pathogens; and 3. stimulating cells to secrete a variety of cytokines that influence the function of other cells involved in adaptive immune responses and innate immune responses.

Cell-mediated immunity is most effective in removing virus-infected cells, but also participates in defending against fungi, protozoans, cancers, and intracellular bacteria. It also plays a major role in transplant rejection.

Tumor induced immune suppression is one of the hallmarks of cancer and a significant hurdle to using immunotherapy to treat cancer. As they develop, tumors adapt to avoid and escape immune detection through several mechanisms. The tumor microenvironment, for example, upregulates many factors that promote the development of suppressive immune cells, such as CD4⁺FoxP3⁺ regulatory T cells (Tregs) (Curiel, 2004a) and myeloid-derived suppressor cells (MDSCs) (Nagaraj and Gabrilovich, 2008). The tumor microenvironment also contributes to the direct suppression of activated CD8+ T cells by releasing immunosuppressive cytokines such as TNF-β (Yang, 2010). Other tumor escape mechanisms that respond to immune pressure are immunoediting, downregulation of MEW class I and alterations in antigen processing and presentation. The use of immunomodulatory agents to counteract tumor induced immune suppression could improve the efficacy of treatments (Yong, 2012).

Since cell-mediated immunity involves the participation of various cell types and is mediated by different mechanisms, several methods could be used to determine efficacy or activity of a cell-mediated immune response. These could be broadly classified into detection of: i) specific antigen presenting cells; ii) specific effector cells and their functions, iii) release of soluble mediators such as cytokines, and iv) detection and counts of immune cells in lymph nodes, lymphatic tissue or at a desired site of immune reaction (e.g. tumor site).

In an embodiment, the methods disclosed herein are capable of enhancing or reducing a cell-mediated immune response by at least 2-fold, at least 3-fold, at least 4-fold, at least 5-fold, at least 6-fold, at least 7-fold, at least 8-fold, at least 9-fold or at least 10-fold, as compared to the cell-mediated immune response of a control not subject to the methods of the invention. In an embodiment, the methods disclosed herein are capable of enhancing or reducing a cell-mediated immune response with only a single administration of the composition. In an embodiment, the methods disclosed herein are capable of enhancing or reducing a cell-mediated immune response with the administration of less total active agent and/or immunomodulatory agent as compared to other methods, such as for example methods involving oral administration of the same active agent and/or immunomodulatory agent.

In an embodiment, the methods and uses disclosed herein are for modulating an antibody immune response in a subject.

An “antibody immune response” or “humoral immune response” (used interchangeably herein), as opposed to cell-mediated immunity, is mediated by secreted antibodies which are produced in the cells of the B lymphocyte lineage (B cells). Such secreted antibodies bind to epitopes, such as for example those on the surfaces of foreign substances, pathogens (e.g. viruses, bacteria, etc.) and/or cancer cells, and flag them for destruction.

As used herein, “humoral immune response” refers to antibody production and may also include, in addition or alternatively, the accessory processes that accompany it, such as for example the generation and/or activation of T-helper 2 (Th2) or T-helper 17 (Th17) cells, cytokine production, isotype switching, affinity maturation and memory cell activation. “Humoral immune response” may also include the effector functions of an antibody, such as for example toxin neutralization, classical complement activation, and promotion of phagocytosis and pathogen elimination. The humoral immune response is often aided by CD4+Th2 cells and therefore the activation or generation of this cell type may also be indicative of the efficacy of a humoral immune response.

A humoral immune response is one of the common mechanisms for combating infectious disease (e.g. to protect against viral or bacterial invaders). However, a humoral immune response can also be useful for combating cancer. B cell mediated responses may target cancer cells through mechanisms which may, in some instances, cooperate with a cytotoxic T cell for maximum benefit. Examples of B cell mediated (e.g. humoral immune response mediated) anti-tumor responses include, without limitation: 1) Antibodies produced by B cells that bind to surface epitopes found on tumor cells or other cells that influence tumorigenesis. Such antibodies can, for example, induce killing of target cells through antibody-dependant cell-mediated cytotoxicity (ADCC) or complement fixation; 2) Antibodies that bind to receptors on tumor cells to block their stimulation and in effect neutralize their effects; 3) Antibodies that bind to factors released by or associated with a tumor or tumor-associated cells to modulate a signaling or cellular pathway that supports cancer; and 4) Antibodies that bind to intracellular targets and mediate anti-tumor activity through a currently unknown mechanism.

One method of evaluating an antibody response is to measure the titers of antibodies. This may be performed using a variety of methods known in the art such as enzyme-linked immunosorbent assay (ELISA) of antibody-containing substances obtained from animals. Without limitation, other assays that may be used include immunological assays (e.g. radioimmunoassay (RIA)), immunoprecipitation assays, and protein blot (e.g. Western blot) assays; and neutralization assays (e.g., neutralization of viral infectivity in an in vitro or in vivo assay).

In an embodiment, the methods disclosed herein are capable of enhancing or reducing an antibody immune response by at least 2-fold, at least 3-fold, at least 4-fold, at least 5-fold, at least 6-fold, at least 7-fold, at least 8-fold, at least 9-fold or at least 10-fold, as compared to the antibody immune response of a control not subject to the methods of the invention. In an embodiment, the methods disclosed herein are capable of enhancing or reducing an antibody immune response with only a single administration of the composition. In an embodiment, the methods disclosed herein are capable of enhancing or reducing an antibody immune response with the administration of less total active agent and/or immunomodulatory agent as compared to other methods, such as for example methods involving oral administration of the same active agent and/or immunomodulatory agent.

In an embodiment, the methods disclosed herein are for modulating an immune response in a subject by the targeted delivery to the lymph nodes of a small molecule drug as described herein. In an embodiment, the small molecule drug is any one or more of epacadostat, rapamycin, doxorubicin, valproic acid, mitoxantrone, vorinostat, cyclophosphamide, irinotecan, cisplatin, methotrexate, tacrolimus or a pharmaceutically acceptable salt of any one thereof.

In an embodiment, the methods disclosed herein are for modulating an immune response in a subject by the targeted delivery to the lymph nodes of an antibody. In an embodiment, the antibody is an anti-CTLA-4 antibody (e.g. ipilimumab, tremelimumab, BN-13, UC10-4F10-11, 9D9 or 9H10). In an embodiment, the antibody is an anti-PD-1 antibody or an anti-PD-L1 antibody (e.g. pembrolizumab, nivolumab, pidilizumab, AMP-224, RMP1-4, J43, atezolizumab, avelumab, BMS-936559 or durvalumab).

In an embodiment, the methods disclosed herein are for modulating an immune response in a subject by the targeted delivery to the lymph nodes of an immunomodulatory agent as described herein. In an embodiment, the immunomodulatory agent is one that binds a checkpoint receptor on the surface of T-lymphocytes, such as for example CTLA-4 or PD-1.

In an embodiment of modulating an immune response in a subject, the active agent and/or immunomodulatory agent counteracts one or more immunosuppressive mechanisms of cancer cells.

In an embodiment of modulating an immune response in a subject, the active agent and/or immunomodulatory agent enhances an immune response to cancer, such as an immune response activated by an anti-cancer vaccine (e.g. a vaccine that delivers a cancer-specific antigen or neoantigen).

In an embodiment of modulating an immune response in a subject, the active agent and/or immunomodulatory agent enhances an immune response to an infectious disease, such as an immune response activated by an antiviral or antibacterial vaccine.

In an embodiment of modulating an immune response in a subject, the active agent and/or immunomodulatory agent reduces an autoimmune response.

In an embodiment of modulating the immune response of a subject, the active agent and/or immunomodulatory agent can increase or decrease a T_{H}1 immune response, a T_{H}2 immune response or both a T_{H}1 immune response and a T_{H}2 immune response.

The methods and uses disclosed herein may also be used for treating or preventing a disease or disorder in a subject by preferentially delivering the active agent and/or immunomodulatory agent to the lymph nodes or lymphoid cells in a lymphatic tissue. “Treating” or “treatment of”, or “preventing” or “prevention of”, as used herein, refers to an approach for obtaining beneficial or desired results. Beneficial or desired results can include, but are not limited to, alleviation or amelioration of one or more symptoms or conditions, diminishment of extent of disease, stabilisation of the state of disease, prevention of development of disease, prevention of spread of disease, delay or slowing of disease progression (e.g. suppression), delay or slowing of disease onset, conferring protective immunity against a disease-causing agent and amelioration or palliation of the disease state. “Treating” or “preventing” can also mean prolonging survival of a patient beyond that expected in the absence of treatment and can also mean inhibiting the progression of disease temporarily or preventing the occurrence of disease, such as by preventing infection in a subject. “Treating” or “preventing” may also refer to a reduction in the size of a tumor mass, reduction in tumor aggressiveness, etc.

As will be appreciated, there may be overlap in treatment and prevention. For example, it is possible to be “treating” a disease in a subject, while at same time “preventing” symptoms or progression of the disease. Moreover, “treating” and “preventing” may overlap in that the treatment of a subject to induce an immune response (e.g. vaccination) may have the subsequent effect of preventing infection by a pathogen or preventing the underlying disease or symptoms caused by infection with the pathogen. These preventive aspects are encompassed herein by expressions such as “treatment of an infectious disease” or “treatment of cancer”.

In an embodiment, the methods disclosed herein may be useful for treating or preventing diseases and/or disorders ameliorated by a cell-mediated immune response or a humoral immune response. In such instances, the methods disclosed herein may be useful for treating and/or preventing the disease or disorder by modulating the respective immune responses.

In an embodiment, the methods disclosed herein may be useful for treating or preventing diseases and/or disorders of lymph nodes or lymphatic tissues or diseases and/or disorders that have aspects that localize to lymph nodes or lymphatic tissues (e.g. metastatic cancers). In such instances, the methods disclosed herein may be useful in the delivery of specific active agents and/or immunomodulatory agents that have a therapeutic effect or activity on the disease or disorder, including but not limited to immunomodulation. For example, the methods may be useful for the targeted delivery of cytotoxic agents, anti-tumor agents, chemotherapeutic agents, antiviral agents, antibacterial agents, anti-inflammatory agents, biological response modifiers, steroids, etc.

In an embodiment, and without limitation, the disease of the lymph nodes or lymphatic tissue may be swollen lymph nodes, lymphoma, Hodgkin's lymphoma, non-Hodgkin's lymphoma, acute lymphoblastic leukemia, lymphatic disease, Castleman's disease, lymphedema, sarcoidosis, cat-scratch disease, tonsillitis, acute tonsillitis, generalized lymphadenopathy, lymphangitis, lymphadenitis, lymphocytosis, streptococcal pharyngitis, Kikuchi disease, Kawasaki disease, adenitis, filariasis, or spleomegaly. The skilled person will be well aware of other diseases and/or disorders of the lymph nodes or lymphatic tissue for which the methods and uses of the present invention may find application.

In an embodiment, the methods disclosed herein may be used for treating and/or preventing an infectious disease, such as caused by a viral infection, in a subject in need thereof. The subject may be infected with a virus or may be at risk of developing a viral infection. Viral infections that may be treated and/or prevented include, without limitation, Cowpoxvirus, Vaccinia virus, Pseudocowpox virus, Human herpesvirus 1 , Human herpesvirus 2, Cytomegalovirus, Human adenovirus A-F, Polyomavirus, Human papillomavirus (HPV), Parvovirus, Hepatitis A virus, Hepatitis B virus, Hepatitis C virus, Human immunodeficiency virus, Orthoreovirus, Rotavirus, Ebola virus, parainfluenza virus, influenza A virus, influenza B virus, influenza C virus, Measles virus, Mumps virus, Rubella virus, Pneumovirus, respiratory syncytial virus (RSV), Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus and varicella. In an embodiment, the viral infection is Human papillomavirus, Ebola virus, respiratory syncytial virus or an influenza virus.

In an embodiment, the methods disclosed herein may be used for treating and/or preventing an infectious disease caused by a non-viral pathogen (such as a bacterium or protozoan) in a subject in need thereof. The subject may be infected with the pathogen or may be at risk of developing an infection by the pathogen. Without limitation, exemplary bacterial pathogens may include Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli O157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica. In a particular embodiment, the bacterial infection is Anthrax. Without limitation, exemplary protozoan pathogens may include those of the genus Plasmodium (Plasmodium falciparum, Plasmodium malariae, Plasmodium vivax, Plasmodium ovale or Plasmodium knowlesi), which cause malaria.

In an embodiment, the methods disclosed herein may be for use in treating and/or preventing cancer in a subject in need thereof. The subject may have cancer or may be at risk of developing cancer.

As used herein, the terms “cancer”, “cancer cells”, “tumor” and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

The methods are not limited to any particular type of cancer. In an embodiment, the cancer may be a primary cancer of the lymph nodes or lymphatic tissue, or a cancer that has spread to the lymph nodes or lymphatic tissue by metastases, e.g. a secondary cancer. In an embodiment, the cancer may be one that is being, or will be, treated by immunotherapy. In an embodiment, the cancer may be one that has adapted to avoid and escape immune detection through an immunosuppressive mechanism.

Without limitation, the cancer may be a carcinoma, adenocarcinoma, lymphoma, leukemia, sarcoma, blastoma, myeloma, or a germ cell cancer. More particularly, the cancer may be glioblastoma, multiple myeloma, ovarian cancer, breast cancer, fallopian tube cancer, prostate cancer, fallopian tube cancer, peritoneal cancer, diffuse large B cell lymphoma or any cancer of the lymph nodes or lymphatic tissue.

In an embodiment, the cancer may be caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukaemia virus-1. In an embodiment, the cancer is one that expresses a cancer-specific antigen (e.g. survivin protein). In an embodiment, the cancer is one that expresses one or more neoantigens. In a particular embodiment, a tumor-specific neoantigen.

In the methods disclosed herein, the amount of any specific active agent and/or immunomodulatory agent may depend on the type of agent, the disease or disorder to be treated, and/or particular characteristics of the subject (e.g. age, weight, sex, immune status, etc.). One skilled in the art can readily determine the amount of active agent and/or immunomodulatory agent needed in a particular application by empirical testing.

Compositions

The compositions of the invention comprise an active agent or immunomodulatory agent as defined herein, one or more lipid-based structures and a hydrophobic carrier. Each of these components is individually defined and described in greater detail herein.

In some embodiments, the compositions may optionally and additionally comprise an antigen, with or without an adjuvanting agent, and in such embodiments the composition may be referred to as a “vaccine composition” or “vaccine”, used interchangeably.

For the methods disclosed herein, it is not required that the compositions comprise an antigen or an adjuvanting agent to achieve targeted delivery of the active agents and/or immunomodulatory agents to lymph nodes or lymphoid cells in a lymphatic tissue. Targeted delivery is independent of classical antigen processing and activation of immune cells, including any necessary assistance by adjuvanting agents. In this regard, in an embodiment, the compositions of the invention do not comprise an antigen, an adjuvant or both. As used herein, by “antigen”, it is meant a compound or substance that induces an antibody and/or cell-mediated immune response (i.e. an immunogen). As used herein, by “adjuvanting agent”, it is meant a compound (i.e. a molecule) that is administered together with an antigen to improve routing and adaptive immune responses to the antigen.

In an embodiment, the active agent and/or immunomodulatory agent for use in the compositions of the invention is a compound, substance or molecule that is not capable of being processed and/or presented to immune cells via classical mechanisms of antigen processing. In this regard, in an embodiment, the active agent and/or immunomodulatory agent is delivered intact to lymph nodes or lymphoid cells in a lymphatic tissue. By “intact”, it is meant that the active agent and/or immunomodulatory agent has not been subject to endosome or proteasome degradation, and the active agent and/or immunomodulatory agent maintains its desired functionality (e.g. biological, pharmaceutical and/or therapeutic activity). In an embodiment, “intact” means that the active agent and/or immunomodulatory agent delivered to the lymph nodes is identical in primary, secondary, tertiary and/or quaternary structure to the active agent and/or immunomodulatory agent as administered.

In an embodiment, the active agent and/or immunomodulatory agent for use in the compositions herein is a compound, substance or molecule that does not directly bind to a major histocompatibility complex (MHC) class I protein, an MHC class II protein, or both. As the skilled person will appreciate, MHC molecules are cell surface proteins that bind polypeptides and display them on the cell surface for recognition by appropriate T cells. For example, immature dendritic cells phagocytose pathogens, degrade their proteins into small pieces, and upon maturation present those fragments at their cell surface using MHC molecules. MHC molecules mediate interactions between immune cells.

A composition as disclosed herein may be administered to a subject in a therapeutically effect amount. As used herein, a “therapeutically effective amount” means an amount of the composition or active agent/immunomodulatory agent contained therein effective to provide a therapeutic, prophylactic or diagnostic benefit to a subject, and/or an amount sufficient to modulate an immune response in a subject. As used herein, to “modulate” an immune response is distinct and different from activating an immune response. By “modulate”, it is meant that the active agents and/or immunomodulatory agents herein enhance or suppress an immune response that is activated by other mechanisms or compounds (e.g. by an antigen or immunogen). In an embodiment, the immune response was activated before the compositions herein are administered. In another embodiment, the immune response may be activated commensurately or subsequently to administration of the compositions described herein.

In some embodiments, a therapeutically effective amount of the composition is an amount capable of inducing a clinical response in a subject in the treatment of a particular disease or disorder. Determination of a therapeutically effective amount of the composition is well within the capability of those skilled in the art, especially in light of the disclosure provided herein. The therapeutically effective amount may vary according to a variety of factors such as the subject's condition, weight, sex and age.

In an embodiment, the compositions disclosed herein are water-free. As used herein, “water-free” means completely or substantially free of water, i.e. the compositions are not emulsions.

By “completely free of water” it is meant that the compositions contain no water at all. In contrast, the term “substantially free of water” is intended to encompass embodiments where the hydrophobic carrier may still contain small quantities of water, provided that the water is present in the non-continuous phase of the carrier. For example, individual components of the composition (e.g. active agents and/or immunomodulatory agents as described herein) may have small quantities of bound water that may not be completely removed by processes such as lyophilization or evaporation and certain hydrophobic carriers may contain small amounts of water dissolved therein. Generally, compositions as disclosed herein that are “substantially free of water” contain, for example, less than about 5%, 4%, 3%, 2%, 1%, 0.5%, 0.1%, 0.05% or 0.01% water on a weight/weight basis of the total weight of the carrier component of the composition.

In the context of a composition for pharmaceutical use, it may be desirable that the final composition be a clear or only slightly hazy solution. In an embodiment, the compositions herein are clear or slightly hazy. As used herein, an embodiment of “slightly hazy” means that the composition displays some turbidity in the solution, but does not have any visible particulates or precipitate or is substantially free of particulates or precipitate. By “substantially free” it is meant that the compositions comprise such a minor content of particulates or precipitate that it would not affect the therapeutic efficacy of the compositions or other pertinent characteristics, such as stability. In an embodiment, the compositions herein are clear. In an embodiment, the compositions herein are substantially free of visible particulates or precipitate. In an embodiment, the compositions herein have no visible particulates or precipitate. The clarity of a composition may be determined visually by the naked eye by observing the solution or by measurement using a spectrophotometer. In an embodiment, the compositions may be visually inspected according to the European Pharmacopoeia (Ph. Eur.), 9^{th} edition, Section 2.9.20.

Active Agents

The compositions disclosed herein are for the targeted delivery of active agents to lymph nodes or lymphoid cells in a lymphatic tissue.

The term “agent” includes any substance, molecule, element, compound, entity, or a combination thereof. It can be a natural product, a synthetic compound, or a combination of two or more substances. Unless otherwise specified, the terms “agent”, “substance” and “compound” are used interchangeably herein.

As used herein, an “active agent” refers to a pharmaceutically or therapeutically active agent or diagnostic agent. The active agent is small molecule drug; an antibody, an antibody mimetic, or a functional equivalent or functional fragment of any one thereof.

Small Molecule Drugs

In an embodiment, the active agent is a small molecule drug. The term “small molecule drug” refers an organic or inorganic compound that may be used to treat, cure, prevent or diagnose a disease, disorder or condition.

As used herein, the term “small molecule” refers to a low molecular weight compound which may be synthetically produced or obtained from natural sources and has a molecular weight of less than 2000 Daltons (Da), less than 1500 Da, less than 1000 Da, less than 900 Da, less than 800 Da, less than 700 Da, less than 600 Da or less than 500 Da. In an embodiment, the small molecule drug has a molecular weight of about 900 Da or less than 900 Da. More particularly, in an embodiment, the small molecule drug has a molecular weight of less than 600 Da, and even more particularly less than 500 Da.

In an embodiment, the small molecule drug has a molecule weight of between about 100 Da to about 2000 Da; about 100 Da to about 1500 Da; about 100 Da to about 1000 Da; about 100 Da to about 900 Da; about 100 Da to about 800 Da; about 100 Da to about 700 Da; about 100 Da to about 600 Da; or about 100 Da to about 500 Da. In an embodiment, the small molecule drug has a molecular weight of about 100 Da, about 150 Da, about 200 Da, about 250 Da, about 300 Da, about 350 Da, about 400 Da, about 450 Da, about 500 Da, about 550 Da, about 600 Da, about 650 Da, about 700 Da, about 750 Da, about 800 Da, about 850 Da, about 900 Da, about 950 Da, about 1000 Da, or about 2000 Da. In an embodiment, the small molecule drug may have a size on the order of 1 nm.

In an embodiment, the small molecule drug is a chemically manufactured active substance or compound (i.e. it is not produced by a biological process). Generally, these compounds are synthesized in the classical way by chemical reactions between different organic and/or inorganic compounds. As used herein, the term “small molecule drug” does not encompass larger structures, such as polynucleotides, proteins and polysaccharides, which are made by a biological process.

In an embodiment, as used herein, the term “small molecule” refers to compounds or molecules that selectively bind specific biological macromolecules and act as an effector, altering the activity or function of the target. Thus, in an embodiment, the small molecule drug is a substance or compound that regulates a biological process in the body of a subject, and more particularly within a cell. The small molecule drug may exert its activity in the form in which it is administered, or the small molecule drug may be a prodrug. In this regard, the term “small molecule drug”, as used herein, encompasses both the active form and the prodrug.

The term “prodrug” refers to a compound or substance that, under physiological conditions, is converted into the therapeutically active agent. In an embodiment, a prodrug is a compound or substance that, after administration, is metabolized in the body of a subject into the pharmaceutically active form (e.g. by enzymatic activity in the body of the subject). A common method for making a prodrug is to include selected moieties that are hydrolyzed under physiological conditions to reveal the pharmaceutically active form.

In an embodiment, and without limitation, the small molecule drug is a cytotoxic agent, an anti-cancer agent, an anti-tumor agent, a chemotherapeutic agent, an anti-neoplastic agent, an antiviral agent, an antibacterial agent, an anti-inflammatory agent, an immunomodulatory agent (e.g. an immune enhancer or suppressor), an immune response checkpoint agent, a biological response modifier, a prodrug, a cytokine, a chemokine, a vitamin, a steroid, a ligand, an analgesic, a radiopharmaceutical, a radioisotope or a dye for visual detection.

The small molecule drug may be any of those described herein, or may be a pharmaceutically acceptable salt thereof. As used herein, the term “pharmaceutically acceptable salt(s)” refers to any salt form of an active agent and/or immunomodulatory agent described herein that are safe and effective for administration to a subject of interest, and that possess the desired biological, pharmaceutical and/or therapeutic activity. Pharmaceutically acceptable salts include salts of acidic or basic groups. Pharmaceutically acceptable acid addition salts may include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Suitable base salts may include, but are not limited to, aluminum, calcium, lithium, magnesium, potassium, sodium, zinc, and diethanolamine salts. A review of pharmaceutically acceptable salts can be found, for example, in Berge, 1977.

In an embodiment, the small molecule drug is an agent that interferes with DNA replication. As used herein, the expression “interferes with DNA replication” is intended to encompass any action that prevents, inhibits or delays the biological process of copying (i.e., replicating) the DNA of a cell. The skilled person will appreciate that there exist various mechanisms for preventing, inhibiting or delaying DNA replication, such as for example DNA cross-linking, methylation of DNA, base substitution, etc. The present disclosure encompasses the use of any agent that interferes with DNA replication. Exemplary, non-limiting embodiments of such agents that may be used are described, for example, in WO2014/153636 and in PCT/CA2017/050539. In an embodiment, the agent that interferes with DNA replication is an alkylating agent, such as for example a nitrogen mustard alkylating agent. In an embodiment, the agent that interferes with DNA replication is cyclophosphamide.

In an embodiment, the small molecule drug is cyclophosphamide, ifosfamide, afosfamide, melphalan, bendamustine, uramustine, palifosfamide, chlorambucil, busulfan, 4-hydroxycyclophosphamide, bis-chloroethylnitrosourea (BCNU), mitomycin C, yondelis, procarbazine, dacarbazine, temozolomide, cisplatin, carboplatin, oxaliplatin, acyclovir, gemcitabine, 5-fluorouracil, cytosine arabinoside, ganciclovir, camptothecin, topotecan, irinotecan, doxorubicin, daunorubicin, epirubicin, idarubicin, etoposide, teniposide, mitoxantrone or pixantrone, or a pharmaceutically acceptable salt of any one thereof.

In an embodiment, the small molecule drug is ifosfamide. Ifosfamide is a nitrogen mustard alkylating agent. The IUPAC name for ifosfamide is N-3-bis(2-chloroethyl)-1,3,2-oxazaphosphinan-2-amide-2-oxide. Ifosfamide is commonly known as Ifex®. The chemical structure of ifosfamide is:

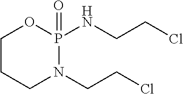 [SMILES: O=P1(NCCCl)OCCCN1CCCl]

In an embodiment, the small molecule drug is palifosfamide. Palifosfamide is an active metabolite of ifosfamide that is covalently linked to the amino acid lysine for stability. Palifosfamide irreversibly alkylates and cross-links DNA through GC base pairs, resulting in irreparable 7-atom inter-strand cross-links; inhibition of DNA replication and/or cell death. Palifosfamide is also known as Zymafos®.

In an embodiment, the small molecule drug is bendamustine. Bendamustine is another nitrogen mustard alkylating agent. The IUPAC name for Bendamustine is 4-[5-[Bis(2-chloroethyl)amino]-1-methylbenzimidazol-2-yl]butanoic acid, and it is commonly referred to as Treakisym®, Ribomustin®, Levact® and Treanda®. The chemical structure of bendamustine is:

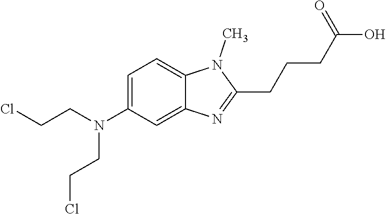 [SMILES: Cn1c(CCCC(=O)O)nc2cc(N(CCCl)CCCl)ccc21]

In an embodiment, the small molecule drug is an immune response checkpoint agent. As used herein, an “immune response checkpoint agent” refers to any compound or molecule that totally or partially modulates (e.g. activates or inhibits) the activity or function of one or more checkpoint molecules (e.g. proteins). Checkpoint molecules are responsible for co-stimulatory or inhibitory interactions of T-cell responses. Checkpoint molecules regulate and maintain self-tolerance and the duration and amplitude of physiological immune responses. Generally, there are two types of checkpoint molecules: stimulatory checkpoint molecules and inhibitory checkpoint molecules.

Stimulatory checkpoint molecules serve a role in enhancing the immune response. Numerous stimulatory checkpoint molecules are known, such as for example and without limitation: CD27, CD28, CD40, CD122, CD137, CD137/4-1BB, ICOS, IL-10, OX40 TGF-beta, TOR receptor, and glucocorticoid-induced TNFR-related protein GITR. In an embodiment, the small molecule drug is an agonist or antagonist of one or more stimulatory checkpoint molecules. In an embodiment, the small molecule drug is an agonist or superagonist of one or more stimulatory checkpoint molecules. The skilled person will be well aware of small molecule drugs that may be used to modulate stimulatory checkpoint molecules.

Inhibitory checkpoint molecules serve a role in reducing or blocking the immune response (e.g. a negative feedback loop). Numerous inhibitory checkpoint proteins are known, such as for example CTLA-4 and its ligands CD80 and CD86; and PD-1 and its ligands PD-L1 and PD-L2. Other inhibitory checkpoint molecules include, without limitation, adenosine A2A receptor (A2AR); B7-H3 (CD276); B7-H4 (VTCN1); BTLA (CD272); killer-cell immunoglobulin-like receptor (KIR); lymphocyte activation gene-3 (LAG3); V-domain Ig suppressor of T cell activation (VISTA) T-cell immunoglobulin domain and mucin domain 3 (TIM-3); and indoleamine 2,3-dioxygenase (IDO), as well as their ligands and/or receptors. In an embodiment, the small molecule drug is an agonist or antagonist of one or more inhibitory checkpoint molecules. In an embodiment, the small molecule drug is an antagonist (i.e. an inhibitor) of one or more inhibitory checkpoint molecules. The skilled person will be well aware of small molecule drugs that may be used to modulate inhibitory checkpoint molecules.

In an embodiment, the small molecule drug is an immune response checkpoint agent that is an inhibitor of Programmed Death-Ligand 1 (PD-L1, also known as B7-H1, CD274), Programmed Death 1 (PD-1, CD279), CTLA-4 (CD154), PD-L2 (B7-DC, CD273), LAG3 (CD223), TIM3 (HAVCR2, CD366), 41BB (CD137), 2B4, A2aR, B7H1, B7H3, B7H4, B- and T-lymphocyte attenuator (BTLA), CD2, CD27, CD28, CD30, CD33, CD40, CD70, CD80, CD86, CD160, CD226, CD276, DR3, GALS, GITR, HVEM, IDO1, IDO2, ICOS (inducible T cell costimulator), Killer inhibitory receptor (KIR), LAG-3, LAIR1, LIGHT, MARCO (macrophage receptor with collageneous structure), phosphatidylserine (PS), OX-40, Siglec-5, Siglec-7, Siglec-9, Siglec-11, SLAM, TIGIT, TIM3, TNF-α, VISTA, VTCN1, or any combination thereof.

In an embodiment, the small molecule drug an immune response checkpoint agent that is an inhibitor of PD-L1, PD-1, CTLA-4 or any combination thereof.

In an embodiment, the small molecule drug may be epacadostat, rapamycin, doxorubicin, valproic acid, mitoxantrone, vorinostat, cyclophosphamide, irinotecan, cisplatin, methotrexate, tacrolimus or a pharmaceutically acceptable salt of any one thereof.

In an embodiment, the small molecule drug is cyclophosphamide or a pharmaceutically acceptable salt thereof. Cyclophosphamide (N,N-bis(2-chloroethyl)-1,3,2-oxazaphosphinan-2-amine 2-oxide). The chemical structure of cyclophosphamide is:

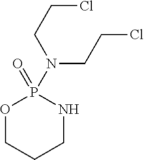 [SMILES: O=P1(N(CCCl)CCCl)NCCCO1]

Cyclophosphamide is also known and referred to under the trade-marks Endoxan®, Cytoxan®, Neosar®, Procytox® and Revimmune®. Cyclophosphamide is a prodrug which is converted to its active metabolites, 4-hydroxy-cyclophosphamide and aldophosphamide, by oxidation by P450 enzymes. Intracellular 4-hydroxy-cyclophosphamide spontaneously decomposes into phosphoramide mustard which is the ultimate active metabolite.

In an embodiment, the small molecule drug is epacadostat:

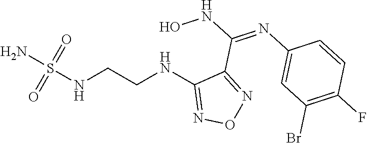 [SMILES: NS(=O)(=O)NCCNc1nonc1/C(=N\c1ccc(F)c(Br)c1)NO]

or a pharmaceutically acceptable salt thereof.

In an embodiment, the small molecule drug is rapamycin:

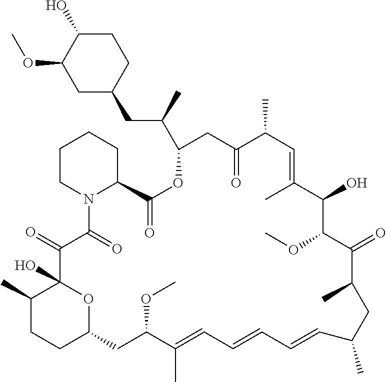 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

or a pharmaceutically acceptable salt thereof.

In an embodiment, the small molecule drug is doxorubicin:

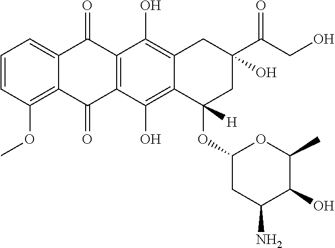 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

or a pharmaceutically acceptable salt thereof.

In an embodiment, the small molecule drug is valproic acid:

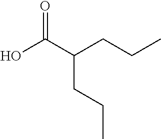

or a pharmaceutically acceptable salt thereof.

In an embodiment, the small molecule drug is mitoxantrone:

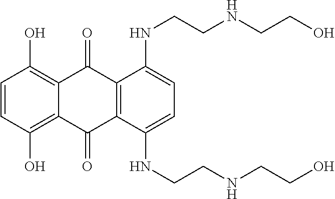 [SMILES: O=C1c2c(O)ccc(O)c2C(=O)c2c(NCCNCCO)ccc(NCCNCCO)c21]

or a pharmaceutically acceptable salt thereof.

In an embodiment, the small molecule drug is vorinostat:

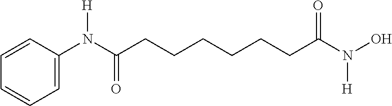 [SMILES: O=C(CCCCCCC(=O)Nc1ccccc1)NO]

or a pharmaceutically acceptable salt thereof.

In an embodiment, the small molecule drug is irinotecan:

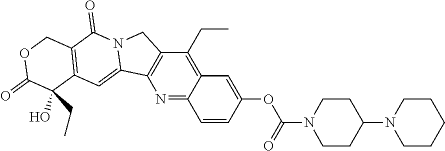 [SMILES: CCc1c2c(nc3ccc(OC(=O)N4CCC(N5CCCCC5)CC4)cc13)-c1cc3c(c(=O)n1C2)COC(=O)[C@]3(O)CC]

or a pharmaceutically acceptable salt thereof.

In an embodiment, the small molecule drug is cisplatin:

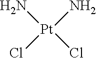

or a pharmaceutically acceptable salt thereof.

In an embodiment, the small molecule drug is methotrexate:

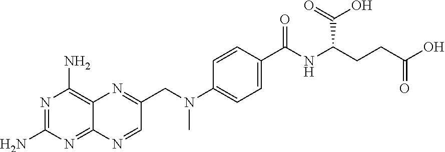 [SMILES: CN(Cc1cnc2nc(N)nc(N)c2n1)c1ccc(C(=O)N[C@@H](CCC(=O)O)C(=O)O)cc1]

or a pharmaceutically acceptable salt thereof.

In an embodiment, the small molecule drug is tacrolimus:

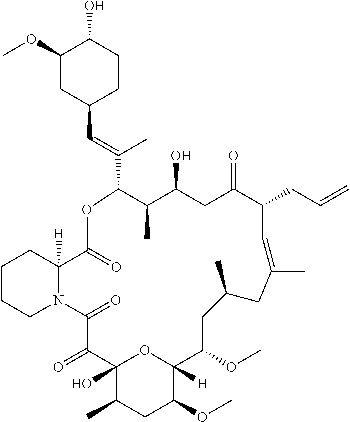 [SMILES: C=CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

or a pharmaceutically acceptable salt thereof.

In an embodiment, the small molecule drug is a shuttle, e.g. a molecular shuttle. As used herein, the term “shuttle” refers to a compound or molecule that can transport other molecules or ions from one location to another. Without limitation, the shuttle may be a peptide that is capable of transporting cargo to cells, such as for example a cell-penetrating peptide (CPP), a peptide transduction domain (PTD) and/or a dendritic cell peptide (DCpep). These types of shuttles are described, for example, in Delcroix, 2010; Zhang, 2016; Zahid, 2012; and Curiel, 2004b. The skilled person will be well aware of other shuttles that may be used in the practice of the invention.

The skilled person would be well aware of other small molecule drugs that may be used in the practice of the invention. As an example, and without limitation, reference is made to DrugBank™ (Wishart, 2017). Version 5.0.11 of DrugBank™, released Dec. 20, 2017, contains 10,990 drug entries, including over 2,500 approved small molecule drugs.

Antibodies, Antibody Mimetics or Functional Equivalents or Fragments

In an embodiment, the active agent is an antibody, a functional equivalent of an antibody or a functional fragment of an antibody.

Broadly, an “antibody” refers to a polypeptide or protein that consists of or comprises antibody domains, which are understood as constant and/or variable domains of the heavy and/or light chains of immunoglobulins, with or without a linker sequence. In an embodiment, polypeptides are understood as antibody domains if they comprise a beta-barrel sequence consisting of at least two beta-strands of an antibody domain structure connected by a loop sequence. Antibody domains may be of native structure or modified by mutagenesis or derivatization, e.g. to modify binding specificity or any other property.

The term “antibody” refers to an intact antibody. In an embodiment, an “antibody” may comprise a complete (i.e. full-length) immunoglobulin molecule, including e.g. polyclonal, monoclonal, chimeric, humanized and/or human versions having full length heavy and/or light chains. The term “antibody” encompasses any and all isotypes and subclasses, including without limitation the major classes of IgA, IgD, IgE, IgG and IgM, and the subclasses IgG1, IgG2, IgG3, IgG4, IgA1 and IgA2. In an embodiment, the antibody is an IgG. The antibody may be one that is naturally occurring or one that is prepared by any means available to the skilled person, such as for example by using animals or hybridomas, and/or by immunoglobulin gene fragment recombinatorial processes. Antibodies are generally described in, for example, Greenfield, 2014).

In an embodiment, the antibody is in an isolated form, meaning that the antibody is substantially free of other antibodies against a different target antigen and/or comprising a different structural arrangement of antibody domains. In an embodiment, the antibody can be an antibody isolated from the serum sample of mammal. In an embodiment, the antibody is in a purified form, such as provided in a preparation comprising only the isolated and purified antibody as the active agent. This preparation may be used in the preparation of a composition of the invention. In an embodiment, the antibody is an affinity purified antibody.

The antibody may be of any origin, including natural, recombinant and/or synthetic sources. In an embodiment, the antibody may be of animal origin. In an embodiment, the antibody may be of mammalian origin, including without limitation human, murine, rabbit and goat. In an embodiment, the antibody may be a recombinant antibody.

In an embodiment, the antibody may be a monoclonal antibody, a polyclonal antibody, a chimeric antibody, a humanized antibody, a human antibody or a fully human antibody. The meaning applied to these terms and the types of antibodies encompassed therein will be well understood by the skilled person.

Briefly, and without limitation, the term “chimeric antibody” as used herein refers to a recombinant protein that contains the variable domains (including the complementarity determining regions (CDRs)) of an antibody derived from one species, such for example a rodent, while the constant domains of the antibody are derived from a different species, such as a human. For veterinary applications, the constant domains of the chimeric antibody may be derived from that of an animal, such as for example a cat or dog.

Without limitation, a “humanized antibody” as used herein refers to a recombinant protein in which the CDRs from an antibody from one species; e.g., a rodent, are transferred from the heavy and light variable chains of the rodent antibody into human heavy and light variable domains, including human framework region (FR) sequences. The constant domains of the humanized antibody are likewise derived from a human antibody.

Without limitation, a “human antibody” as used herein refers to an antibody obtained from transgenic animals (e.g. mice) that have been genetically engineered to produce specific human antibodies in response to antigenic challenge. In this technique, elements of the human heavy and light chain loci are introduced into strains of mice derived from embryonic stem cell lines that contain targeted disruptions of the endogenous heavy chain and light chain loci. The transgenic animal can synthesize human antibodies specific for human antigens, and the animal can be used to produce human antibody-secreting hybridomas. Methods for obtaining human antibodies from transgenic mice are described e.g. by Green, 1994; Lonberg, 1994; and Taylor, 1994. A fully human antibody also can be constructed by genetic or chromosomal transfection methods, as well as phage display technology, all of which are known in the art. (See, e.g., McCafferty, 1990, for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, 1993. Human antibodies may also be generated by in vitro activated B cells (see, e.g., U.S. Pat. Nos. 5,567,610 and 5,229,275).

As used herein, the term “functional fragment”, with respect to an antibody, refers to an antigen-binding portion of an antibody. In this context, by “functional” it is meant that the fragment maintains its ability to bind to the target antigen. In an embodiment, the binding affinity may be equivalent to, or greater than, that of parent antibody. In an embodiment, the binding affinity may be less than the parent antibody, but nevertheless the functional fragment maintains a specificity and/or selectivity for the target antigen.

In an embodiment, in addition to the functional fragment maintaining its ability to bind to the target antigen of the parent antibody, the functional fragment also maintains the effector function of the antibody, if applicable (e.g. activation of the classical complement pathway; antibody dependent cellular cytotoxicity (ADCC); other downstream signalling processes).

Functional fragments of antibodies include, without limitation, a portion of an antibody such as a F(ab′)₂, a F(ab)₂, a Fab′, a Fab, a Fab₂, a Fab₃, a single domain antibody (e.g. a Dab or VHHs) and the like, including half-molecules of IgG4 (van der Neut Kolfschoten, 2007). Regardless of structure, a functional fragment of an antibody binds with the same antigen that is recognized by the intact antibody. The term “functional fragment”, in relation to antibodies, also includes isolated fragments consisting of the variable regions, such as the “Fv” fragments consisting of the variable regions of the heavy and light chains and recombinant single chain polypeptide molecules in which light and heavy chain variable regions are connected by a peptide linker (“scFv proteins”). As used herein, the term “functional fragment” does not include fragments such as Fc fragments that do not contain antigen-binding sites.

Antibody fragments, such as those described herein, can be incorporated into single domain antibodies (e.g. nanobodies), single-chain antibodies, maxibodies, evibodies, minibodies, intrabodies, diabodies, triabodies, tetrabodies, vNAR, bis-scFv and other like structures (see e.g. Hollinger and Hudson, 2005). Antibody polypeptides including fibronectin polypeptide monobodies, also are disclosed in U.S. Pat. No. 6,703,199. Other antibody polypeptides are disclosed in U.S. Patent Publication No. 20050238646.

Another form of a functional fragment is a peptide comprising one or more CDRs of an antibody or one or more portions of the CDRs, provided the resultant peptide retains the ability to bind the target antigen.

A functional fragment may be a synthetic or genetically engineer protein. For example, functional fragments include isolated fragments consisting of the light chain variable region, “Fv” fragments consisting of the variable regions of the heavy and light chains, and recombinant single chain polypeptide molecules which light and heavy regions are connected by a peptide linker (scFv proteins).

As used herein, the terms “antibody” and “functional fragments” of antibodies encompass any derivatives thereof. By “derivatives” it is meant any modification to the antibody or functional fragment, including both modifications that occur naturally (e.g. in vivo) or that are artificially introduced (e.g. by experimental design). Non-limiting examples of such modifications include, for example, sequence modifications (e.g. amino acid substitutions, insertions or deletions), post-translational modifications (e.g. phosphorylation, N-linked glycosylation, O-linked glycosylation, acetylation, hydroxylation, methylation, ubiquitylation, amidation, etc.), or any other covalent attachment or incorporation otherwise of a heterologous molecule (e.g. a polypeptide, a localization signal, a label, a targeting molecule, etc.). In an embodiment, modification of the antibody or functional fragment thereof may be made to generate a bispecific antibody or fragment (i.e. having more than one antigen-binding specificity) or a bifunctional antibody or fragment (i.e. having more than one effector function).

As used herein, a “functional equivalent” in the context of an antibody refers to a polypeptide or other compound or molecule having similar binding characteristics as an antibody to a particular target, but not necessarily being a recognizable “fragment” of an antibody. In an embodiment, a functional equivalent is a polypeptide having an equilibrium dissociation constant (K_{D}) for a particular target in the range of 10⁻⁷ to 10⁻¹². In an embodiment, the functional equivalent has a K_{D} for a particular target of 10⁻⁸ or lower. In an embodiment, the functional equivalent has a K_{D} for a particular target of 10⁻¹⁰ or lower. In an embodiment, the functional equivalent has a K_{D} for a particular target of 10⁻¹¹ or lower. In an embodiment, the functional equivalent has a K_{D} for a particular target of 10⁻¹² or lower. The equilibrium constant (K_{D}) as defined herein is the ratio of the dissociation rate (K-off) and the association rate (K-on) of a compound to its target.

In an embodiment, the antibody, functional fragment thereof or functional equivalent thereof, is one that is preferentially targeted to lymph nodes or lymphoid cells in a lymphatic tissue to exert its pharmacological and/or therapeutic activity. For example and without limitation, the antibody, functional fragment thereof or functional equivalent thereof may be one that binds to an immune cell in lymph nodes or lymphatic tissue, binds to a desired target expressed or found in lymph nodes or lymphatic tissue (e.g. immune stimulatory or inhibitory molecules) and/or binds to cells, proteins, polypeptides or other targets that may be sequestered or delivered to lymph nodes or lymphatic tissue.

In an embodiment, the antibody, functional fragment thereof or functional equivalent thereof, is one that binds a target on an immune cell, binds a protein or polypeptide produced by an immune cell, or binds a protein or polypeptide that interacts with or exerts a function upon immune cells (e.g. a ligand).

In an embodiment, the antibody, functional fragment thereof or functional equivalent thereof, is one that has an immunomodulatory activity or function. By “immunomodulatory activity or function”, it is meant that the antibody, functional fragment thereof or functional equivalent thereof can enhance (upregulate), suppress (downregulate), direct, redirect or reprogram the immune response.

In an embodiment, the antibody, functional fragment thereof or functional equivalent thereof, is one that binds to a stimulatory checkpoint molecule and/or an inhibitory checkpoint molecule, such has for example, and without limitation, those described herein. In an embodiment, the antibody, functional fragment thereof or functional equivalent thereof, is an agonist or an antagonist of a stimulatory checkpoint molecule and/or an inhibitory checkpoint molecule. In an embodiment, the antibody, functional fragment thereof or functional equivalent thereof, is an antagonist of an inhibitory checkpoint molecule. In an embodiment, the antibody, functional fragment thereof or functional equivalent thereof, is an agonist or super agonist of a stimulatory checkpoint molecule.

In an embodiment, the antibody is an anti-CTLA-4 antibody, a functional fragment thereof or a functional equivalent thereof, or any combination thereof. CTLA-4 (CD152) is a protein receptor that, functioning as an immune checkpoint, downregulates immune responses. In an embodiment, the anti-CTLA-4 antibody inhibits CTLA-4 activity or function, thereby enhancing immune responses. In an embodiment, the anti-CTLA-4 antibody is ipilimumab (Bristol-Myers Squibb), tremelimumab (Pfizer; AstraZeneca) or BN-13 (BioXCell). In another embodiment, the anti-CTLA-4 antibody is UC10-4F10-11, 9D9 or 9H10 (BioXCell) or a human or humanized counterpart thereof.

In an embodiment, the antibody is an anti-PD-1 antibody, a functional fragment thereof or a functional equivalent thereof, or any combination thereof. PD-1 (CD279) is a cell surface receptor that, functioning as an immune checkpoint, downregulates immune responses and promotes self tolerance. In an embodiment, the PD-1 antibody is nivolumab (Opdivo™; Bristol-Myers Squibb). In an embodiment, the PD-1 antibody is pembrolizumab (Keytruda™; Merck). In an embodiment, the PD-1 antibody is pidilizumab (Cure Tech). In an embodiment, the anti-PD-1 antibody is AMP-224 (MedImmune & GSK). In an embodiment, the anti-PD-1 antibody is RMP1-4 or J43 (BioXCell) or a human or humanized counterpart thereof.

In an embodiment, the antibody is an anti-PD-L1 antibody, a functional fragment thereof or a functional equivalent thereof, or any combination thereof. PD-L1 is a ligand of the PD-1 receptor, and binding to its receptor transmits an inhibitory signal that reduces proliferation of CD8+ T cells and can also induce apoptosis. In an embodiment, the PD-L1 antibody is BMS-936559 (Bristol Myers Squibb). In an embodiment, the PD-L1 antibody is atezolizumab (MPDL3280A; Roche). In an embodiment, the PD-L1 antibody is avelumab (Merck & Pfizer). In an embodiment, the PD-L1 antibody is durvalumab (MEDI4736; MedImmune/AstraZeneca).

In other embodiments, and without limitation, the antibody, functional fragment or functional equivalent thereof, may be an anti-PD1 or anti-PDL1 antibody, such as for example those disclosed in WO 2015/103602.

In an embodiment, the active agent is an antibody mimetic, a functional equivalent of an antibody mimetic, or a functional fragment of an antibody mimetic.

As used herein, the term “antibody mimetic” refers to compounds which, like antibodies, can specifically and/or selectively bind antigens or other targets, but which are not structurally related to antibodies. Antibody mimetics are usually artificial peptides or proteins, but they are not limited to such embodiments. Typically, antibody mimetics are smaller than antibodies, with a molar mass of about 3-20 kDa (whereas antibodies are generally about 150 kDa). Non-limiting examples of antibody mimetics include peptide aptamers, affimers, affilins, affibodies, affitins, alphabodies, anticalins, avimers, DARPins™, fynomers, Kunitz domain peptides, nanoCLAMPs™, affinity reagents and scaffold proteins. Nucleic acids and small molecules may also be antibody mimetics.

The term “peptide aptamer”, as used herein, refers to peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is a commonly used scaffold protein, the variable peptide loop being inserted within the redox-active site, which is a -Cys-Gly-Pro-Cys-loop in the wild protein, the two cysteins lateral chains being able to form a disulfide bridge. Peptide aptamer selection can be made using different systems, but the most widely used is currently the yeast two-hybrid system.

The term “affimer”, as used herein, represents an evolution of peptide aptamers. An affimer is a small, highly stable protein engineered to display peptide loops which provides a high affinity binding surface for a specific target protein or antigen. Affimers can have the same specificity advantage of antibodies, but are smaller, can be chemically synthesized or chemically modified and have the advantage of being free from cell culture contaminants. Affimers are proteins of low molecular weight, typically 12 to 14 kDa, derived from the cysteine protease inhibitor family of cystatins. The affimer scaffold is a stable protein based on the cystatin protein fold. It displays two peptide loops and an N-terminal sequence that can be randomised to bind different target proteins with high affinity and specificity.

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity is effected, for example, by phage display or ribosome display techniques. Depending on the scaffold, affilins have a molecular weight of approximately 10 kDa (ubiquitin) or 20 kDa (gamma-B crystalline). As used herein, the term affilin also refers to di- or multimerised forms of affilins (Weidle, 2013).

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomization of 13 amino acids located in two alpha-helices involved in the binding activity of the parent protein domain (Feldwisch and Tolmachev, 2012). In an embodiment, it is an Affibody™ sourced from Affibody AB, Stockholm, Sweden.

A “affitin” (also known as nanofitin) is an antibody mimetic protein that is derived from the DNA binding protein Sac7d of Sulfolobus acidocaldarius. Affitins usually have a molecular weight of around 7 kDa and are designed to specifically bind a target molecule by randomising the amino acids on the binding surface (Mouratou, 2012). In an embodiment, the affitin is as described in WO 2012/085861.

The term “alphabody”, as used herein, refers to small 10 kDa proteins engineered to bind to a variety of antigens. Alphabodies are developed as scaffolds with a set of amino acid residues that can be modified to bind protein targets, while maintaining correct folding and thermostability. The alphabody scaffold is computationally designed based on coiled-coil structures, but it has no known counterpart in nature. Initially, the scaffold was made of three peptides that associated non-covalently to form a parallel coiled-coil trimer (US Patent Publication No. 20100305304), but was later redesigned as a single peptide chain containing three a-helices connected by linker regions (Desmet, 2014).

The term “anticalin”, as used herein, refers to an engineered protein derived from a lipocalin (Beste, 1999); Gebauer and Skerra, 2009). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

The term “avimer” (avidity multimers), as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity can be selected, for example, by phage display techniques. The binding specificity of the different A-domains contained in an avimer may, but does not have to be identical (Weidle, 2013).

The term “DARPin™”, as used herein, refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “Fynomer™”, as used herein, refers to a non-immunoglobulin-derived binding polypeptide derived from the human Fyn SH3 domain. Fyn SH3-derived polypeptides are well-known in the art and have been described, e.g. in Grabulovski, 2007; WO 2008/022759; Bertschinger, 2007; Gebauer and Skerra, 2009; and Schlatter, 2012).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity can be selected by display techniques such as phage display (Weidle, 2013).

The term “monobody” (also referred to as “adnectin”), as used herein, relates to a molecule based on the 10th extracellular domain of human fibronectin III (10Fn3), which adopts an Ig- like (3-sandwich fold of 94 residues with 2 to 3 exposed loops, but lacks the central disulphide bridge (Gebauer and Skerra, 2009). Monobodies with the desired target specificity can be genetically engineered by introducing modifications in specific loops of the protein. In an embodiment, the monobody is an ADNECTIN™ (Bristol-Myers Squibb, New York, N.Y.).

The term “nanoCLAMP” (CLostridal Antibody Mimetic Proteins), as used herein, refers to affinity reagents that are 15 kDa proteins having tight, selective and gently reversible binding to target molecules. The nanoCLAMP scaffold is based on an IgG-like, thermostable carbohydrate binding module family 32 (CBM32) from a Clostridium perfringens hyaluronidase (Mu toxin). The shape of nanoCLAMPs approximates a cylinder of approximately 4 nm in length and 2.5 nm in diameter, roughly the same size as a nanobody. nanoCLAMPs to specific targets are generated by varying the amino acid sequences and sometimes the length of three solvent exposed, adjacent loops that connect the beta strands making up the beta-sandwich fold, conferring binding affinity and specificity for the target (Suderman, 2017).

The term “affinity reagent”, as used herein, refers to any compound or substance that binds to a larger target molecule to identify, track, capture or influence its activity. Although antibodies and peptide aptamers are common examples, many different types of affinity reagents are available to the skilled person. In an embodiment, the affinity reagent is one that provides a viable scaffold that can be engineered to specifically bind a target (e.g. Top7 is a scaffold engineered specifically to bind CD4; Boschek, 2009).

The term “scaffold proteins”, as used herein, refers polypeptides or proteins that interact and/or bind with multiple members of a signalling pathway. They are regulators of many key signalling pathways. In such pathways, they regulate signal transduction and help localize pathway components. Herein, they are encompassed by the term “antibody mimetics” for their ability to specifically and/or selectively bind target proteins, much like antibodies. In addition to their binding function and specificity, scaffold proteins may also have enzymatic activity. Exemplary scaffold proteins include, without limitation, kinase suppressor of Ras 1 (KNS), MEK kinase 1 (MEKK1), B-cell lymphoma/leukemia 10 (BCL-10), A-kinase-anchoring protein (AKAP), Neuroblast differentiation-associated protein AHNAK, HOMER1, pellino proteins, NLRP family, discs large homolog 1 (DLG1) and spinophillin (PPP1R9B).

Other embodiments of antibody mimetics include, without limitation, Z domain of Protein A, Gamma B crystalline, ubiquitin, cystatin, Sac7D from Sulfolobus acidocaldarius, lipocalin, A domain of a membrane receptor, ankyrin repeat motive, SH3 domain of Fyn, Kunits domain of protease inhibitors, the 10^{th} type III domain of fibronectin, 3- or 4-helix bundle proteins, an armadillo repeat domain, a leucine-rich repeat domain, a PDZ domain, a SUMO or SUMO-like domain, an immunoglobulin-like domain, phosphotyrosine-binding domain, pleckstrin homology domain, or src homology 2 domain.

As used herein, the term “functional fragment”, with respect to an antibody mimetic, refers any portion or fragment of an antibody mimetic that maintains the ability to bind to its target molecule. The functional fragment of an antibody mimetic may be, for example, a portion of any of the antibody mimetics as described herein. In an embodiment, the binding affinity may be equivalent to, or greater than, that of parent antibody mimetic. In an embodiment, the binding affinity may be less than the parent antibody mimetic, but nevertheless the functional fragment maintains a specificity and/or selectivity for the target antigen.

In an embodiment, in addition to the functional fragment of an antibody mimetic maintaining its ability to bind to the target molecule of the parent antibody mimetic, the functional fragment also maintains the effector function of the antibody mimetic, if applicable (e.g. downstream signalling).

As used herein, a “functional equivalent” in the context of an antibody mimetic refers to a polypeptide or other compound or molecule having similar binding characteristics to an antibody mimetic, but not necessarily being a recognizable “fragment” of an antibody mimetic. In an embodiment, a functional equivalent is a polypeptide having an equilibrium dissociation constant (K_{D}) for a particular target in the range of 10⁻⁷ to 10⁻¹². In an embodiment, the functional equivalent has a K_{D} for a particular target of 10⁻⁸ or lower. In an embodiment, the functional equivalent has a K_{D} for a particular target of 10⁻¹⁰ or lower. In an embodiment, the functional equivalent has a K_{D} for a particular target of 10⁻¹¹ or lower. In an embodiment, the functional equivalent has a K_{D} for a particular target of 10⁻¹² or lower. The equilibrium constant (K_{D}) as defined herein is the ratio of the dissociation rate (K-off) and the association rate (K-on) of a compound to its target.

In an embodiment, the antibody mimetic, functional fragment thereof or functional equivalent thereof, is one that is preferentially targeted to lymph nodes or lymphoid cells in a lymphatic tissue to exert its pharmacological and/or therapeutic activity. For example and without limitation, the antibody mimetic, functional fragment thereof or functional equivalent thereof may be one that binds to an immune cell in lymph nodes or lymphatic tissue, binds to a desired target expressed or found in lymph nodes or lymphatic tissue (e.g. immune stimulatory or inhibitory molecules) and/or binds to cells, proteins, polypeptides or other targets that may be sequestered or delivered to lymph nodes or lymphatic tissue.

In an embodiment, the antibody mimetic, functional fragment thereof or functional equivalent thereof, is one that binds a target on an immune cell, binds a protein or polypeptide produced by an immune cell, or binds a protein or polypeptide that interacts with or exerts a function upon immune cells (e.g. a ligand).

In an embodiment, the antibody mimetic, functional fragment thereof or functional equivalent thereof, is one that has an immunomodulatory activity or function. In an embodiment, the antibody mimetic, functional fragment thereof or functional equivalent thereof, is one that binds to a stimulatory checkpoint molecule and/or an inhibitory checkpoint molecule, such has for example, and without limitation, those described herein. In an embodiment, the antibody mimetic, functional fragment thereof or functional equivalent thereof, is an agonist or an antagonist of a stimulatory checkpoint molecule and/or an inhibitory checkpoint molecule. In an embodiment, the antibody mimetic, functional fragment thereof or functional equivalent thereof, is an antagonist of an inhibitory checkpoint molecule (e.g. CTLA-4, PD-1 or PD-L1). In an embodiment, the antibody mimetic, functional fragment thereof or functional equivalent thereof, is an agonist or super agonist of a stimulatory checkpoint molecule.

The amount of any specific active agent as described herein may depend on the type of agent (e.g. small molecule drug, antibody, functional fragment, etc.). One skilled in the art can readily determine the amount of active agent needed in a particular application by empirical testing.

Immunomodulatory Agent

As used herein, an “immunomodulatory agent” is a compound or molecule that modulates the activity and/or effectiveness of an immune response. “Modulate”, as used herein, means to enhance (upregulate), suppress (downregulate), direct, redirect or reprogram an immune response. The term “modulate” is not intended to mean activate or induce. By this, it is meant that the immunomodulatory agent modulates (enhances, reduces or directs) an immune response that is activated, initiated or induced by a particular substance (e.g. an antigen), but the immunomodulatory agent is not itself the substance against which the immune response is directed, nor is the immunomodulatory agent derived from that substance.

In an embodiment, the immunomodulatory agent is one that modulates myeloid cells (monocytes, macrophages, dendritic cells, magakaryocytes and granulocytes) or lymphoid cells (T cells, B cells and natural killer (NK) cells). In a particular embodiment, the immunomodulatory agent is one that modulates only lymphoid cells. In an embodiment, the immunomodulatory agent is a therapeutic agent that, when administered, stimulates immune cells to proliferate or become activated.

In an embodiment, the immunomodulatory agent is one that enhances the immune response. The immune response may be one that was previously activated or initiated, but is of insufficient efficacy to provide an appropriate or desired therapeutic benefit. Alternatively, the immunomodulatory agent may be provided in advance to prime the immune system, thereby enhancing a subsequently activated immune response.

In an embodiment, an immunomodulatory agent that enhances the immune response may be selected from cytokines (e.g. certain interleukins and interferons), stem cell growth factors, lymphotoxins, co-stimulatory molecules, hematopoietic factors, colony stimulating factors, erythropoietins, thrombopoietins, and the like, and synthetic analogs of these molecules.

In an embodiment, an immunomodulatory agent that enhances the immune response may be selected from: lymphotoxins, such as tumor necrosis factor (TNF); hematopoietic factors, such as interleukin (IL); colony stimulating factor, such as granulocyte-colony stimulating factor (G-CSF) or granulocyte macrophage-colony stimulating factor (GM-CSF); interferon, such as interferons-alpha, -beta or -lamda; and stem cell growth factor, such as that designated “SI factor”.

Included among the cytokines are growth hormones, such as human growth hormone, N-methionyl human growth hormone, and bovine growth hormone; parathyroid hormone; thyroxine; insulin; proinsulin; relaxin; prorelaxin; glycoprotein hormones, such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors, such as NGF-beta; platelet-growth factor; transforming growth factors (TGFs), such as TGF-alpha and TGFP; insulin-like growth factor-I and -II; erythropoietin (EPO); osteoinductive factors; interferons, such as interferon-alpha, -beta, and -gamma; colony stimulating factors (CSFs), such as macrophage-CSF (M-CSF); interleukins (ILs), such as IL-1, IL-lalpha, IL-2, IL-3, IL-4, IL-5, IL-6, IL-7, IL-8, IL- 9, IL-10, IL-1 1, IL-12; IL-13, IL-14, IL-15, IL-16, IL-17, IL-18, IL-21, IL-25, LIF, kit-ligand or FLT-3, angiostatin, thrombospondin, endostatin and tumor necrosis factor.

In an embodiment, the immunomodulatory agent can be an agent which modulates a checkpoint inhibitor. Immune checkpoint proteins are signaling proteins that play a role in regulating immune response. Some checkpoint inhibitors are receptors located on the surface of a cell that respond to extracellular signaling. For example, many checkpoints are initiated by ligand-receptor interactions. When activated, inhibitory checkpoint proteins produce an anti-inflammatory response that can include activation of regulatory T cells and inhibition of cytotoxic or killer T cells. Cancer cells have been shown to express inhibitory checkpoint proteins as a way to avoid recognition by immune cells. Accordingly, inhibitors of inhibitory checkpoint proteins (i.e. “immune checkpoint inhibitors”) can be used to activate the immune system in an individual to kill cancer cells (see e.g. Pardoll, 2012).

In an embodiment, the immunomodulatory agent is any compound, molecule or substance that is an immune checkpoint inhibitor, including but not limited to, an inhibitor of an immune checkpoint protein selected from Programmed Death-Ligand 1 (PD-L1, also known as B7-H1, CD274), Programmed Death 1 (PD-1, CD279), CTLA-4 (CD154), PD-L2 (B7-DC, CD273), LAG3 (CD223), TIM3 (HAVCR2, CD366), 41BB (CD137), 2B4, A2aR, B7H1, B7H3, B7H4, B- and T-lymphocyte attenuator (BTLA), CD2, CD27, CD28, CD30, CD33, CD40, CD70, CD80, CD86, CD160, CD226, CD276, DR3, GALS, GITR, HVEM, ID01, IDO2, ICOS (inducible T cell costimulator), Killer inhibitory receptor (KIR), LAG-3, LAIR1, LIGHT, MARCO (macrophage receptor with collageneous structure), phosphatidylserine (PS), OX-40, Siglec-5, Siglec-7, Siglec-9, Siglec-11, SLAM, TIGIT, TIM3, TNF-a, VISTA, VTCN1, or any combination thereof.

In an embodiment, the immunomodulatory agent is any compound, molecule or substance that inhibits or blocks CTLA-4. CTLA-4 signaling inhibits T-cell activation, particularly during strong T-cell responses. CTLA-4 blockade using CTLA-4 inhibitors, such as anti-CTLA-4 monoclonal antibodies, has great appeal because suppression of inhibitory signals results in the generation of an antitumor T-cell response. Both clinical and preclinical data indicate that CTLA-4 blockade results in direct activation of CD4+ and CD8+ effector cells, and anti-CTLA-4 monoclonal antibody therapy has shown promise in a number of cancers.

In an embodiment, the immunomodulatory agent is any compound, molecule or substance that inhibits or blocks PD-1. Like CTLA-4 signaling, PD-1/PD-L1 modulates T-cell response. Tregs that express PD-1 have been shown to have an immune inhibitor response and PD-1/PD-L1 expression is thus thought to play a role in self-tolerance. In the context of cancer, tumor cells over express PD-1 and PD-L1 in order to evade recognition by the immune system. Anti-cancer therapy that blocks the PD-L1/PD-1 increases effector T cell activity and decreases suppressive Treg activity which allows recognition and destruction of the tumor by an individual's immune system.

Various checkpoint inhibitors may be used. For example, the checkpoint inhibitor may be an antibody that binds to and antagonizes an inhibitory checkpoint protein. Exemplary antibodies include anti-PD1 antibodies (pembrolizumab, nivolumab, pidilizumab, AMP-224, RMP1-4 or J43), anti-PD-L1 antibodies (atezolizumab, avelumab, BMS-936559 or durvalumab), anti-CTLA-4 antibodies (ipilimumab, tremelimumab, BN-13, UC10-4F10-11, 9D9 or 9H10) and the like. In some embodiments, the checkpoint inhibitor may be a small molecule or an RNAi that targets an inhibitory checkpoint protein. In some embodiments, the checkpoint inhibitor may be a peptidomimetic or a polypeptide.

In an embodiment, the immunomodulatory agent may be an immune costimulatory molecule agonist. Immune costimulatory molecules are signaling proteins that play a role in regulating immune response. Some immune costimulatory molecules are receptors located on the surface of a cell that respond to extracellular signaling. When activated, immune costimulatory molecules produce a pro-inflammatory response that can include suppression of regulatory T cells and activation of cytotoxic or killer T cells. Accordingly, immune costimulatory molecule agonists can be used to activate the immune system in an individual to kill cancer cells.

Exemplary immune costimulatory molecules include any of CD27, CD28, CD40, CD122, CD137, CD137/4-1BB, ICOS, IL-10, OX40 TGF-beta, TOR receptor, and glucocorticoid-induced TNFR-related protein GITR. For example OX40 stimulation suppresses Treg cell function while enhancing effector T cell survival and activity, thereby increasing anti-tumor immunity.

In an embodiment, the immunomodulatory agent is any compound, molecule or substance that is an agonist of a costimulatory immune molecule, including, but not limited to, a costimulatory immune molecule selected from CD27, CD28, CD40, CD122, CD137, CD137/4-1BB, ICOS, IL-10, OX40 TGF-beta, TOR receptor, and glucocorticoid-induced TNFR-related protein GITR.

Various immune costimulatory molecule agonists may be used. For example, the immune costimulatory molecule agonist may be an antibody that binds to and activates an immune costimulatory molecule. In further embodiments, the immune costimulatory molecule agonist may be a small molecule that targets and activates an immune costimulatory molecule.

In an embodiment, the immunomodulatory agent is any compound, molecule or substance that is an immunosuppressive agent. By “immunosuppressive agent”, it is meant that the compound, molecule or substance reduces (downregulates) the activity and/or efficacy of the immune response, or directs, redirects or reprograms the immune response in a manner that alleviates an undesired result (e.g. an autoimmune response or allergy). There are many different types of immunosuppressive agent, including, without limitation, calcineurin inhibitors, interleukin inhibitors, selective immunosuppressants and THF-alpha inhibitors.

In an embodiment, and without limitation, the immunomodulatory agent may be an immunosuppressant selected from 5-fluorouracil, 6-thioguanine, adalimumab, anakinra, Atgam, abatacept, alefacept, azathioprine, basiliximab, belatacept, belimumab, benralizumab, brodalumab, canakinumab, certolizumab, chlorambucil, cyclosporine, daclizumab, dimethyl fumerate, dupilumab, eculizumab, efalizumab, ethanercept, everolimus, fingolimod, golimumab, guselkumab, imiquimod, infliximab, ixekizumab, leflunomide, lenlidomide, mechlorethamine, mepolizumab, methotrexate, muromonab-cd3, mycophenolate mofetil, mycophenolic acid, natallizumab, omalizumab, pomalidomide, pimecrolimus, reslizumab, rilonacept, sarilumab, secukinumab, siltuximab, sirolimus, tacrolimus, teriflunomide, thalidomide, Thymoglobulin, tocilizumab, ustekinumab and vedolizumab.

In an embodiment, the immunomodulatory agent is any compound, molecule or substance that is an immunosuppressive cytotoxic drug. In an embodiment, the immunosuppressive cytotoxic drug is a glucocorticoid, a cytostatic (e.g. alkylating agents, antimetabolites), an antibody, a drug acting on immunophilins, an interferon, an opioid, or a TNF binding protein. Immunosuppressive cytotoxic drugs include, without limitation, nitrogen mustards (e.g. cyclophosphamide), nitrosoureas, platinum compounds, folic acid analogs (e.g. methotrexate), purine analogs (e.g. azathioprine and mercaptopurine), pyrimidine analogs (e.g. fluorouracil), protein synthesis inhibitors, cytotoxic antibiotics (e.g. dactinomycin, anthracyclines, mitomycin C, bleomycin and mithramycin), cyclosporine, tacrolimus, sirolimus/rapamycin, everolimus, prednisone, dexamethasone, hydrocortisone, mechlorethamine, clorambucil, mycopholic acid, fingolimod, myriocin, infliximab, etanercept, or adalimumab.

In an embodiment, the immunomodulatory agent is an anti-inflammatory agent. In one embodiment, the anti-inflammatory agent is a non-steroidal anti-inflammatory agent. In an embodiment, the non-steroidal anti-inflammatory agent is a Cox-1 and/or Cox-2 inhibitor. In an embodiment, anti-inflammatory agent includes, without limitation, aspirin, salsalate, diflunisal, ibuprofen, fenoprofen, flubiprofen, fenamate, ketoprofen, nabumetone, piroxicam, naproxen, diclofenac, indomethacin, sulindac, tolmetin, etodolac, ketorolac, oxaprozin, or celecoxib. In an embodiment, the anti-inflammatory agent is a steroidal anti-inflammatory agent. In an embodiment, the steroidal anti-inflammatory agent is a corticosteroid.

In an embodiment, the immunomodulatory agent is an anti-rheumatic agent. In an embodiment, the anti-rheumatic agent is a non-steroidal anti-inflammatory agent. In an embodiment, the anti-rheumatic agent is a corticosteroid. In an embodiment, the corticosteroid is prednisone or dexamethasone. In an embodiment, the anti-rheumatic agent is a disease modifying anti-rheumatic drug. In an embodiment, disease modifying anti-rheumatic drugs include but are not limited to chloroquine, hydroxychloroquine, methotrexate, sulfasalazine, cyclosporine, azathioprine, cyclophosphamide, azathioprine, sulfasalazine, penicillamine, aurothioglucose, gold sodium thiomalate, or auranofin. In an embodiment, the anti-rheumatic agent is an immunosuppressive cytotoxic drug. In one embodiment, immunosuppressive cytotoxic drugs include but are not limited to methotrexate, mechlorethamine, cyclophosphamide, chlorambucil or azathioprine.

In an embodiment, the immunomodulatory agent is any one or more of the active agents as described herein (e.g. a small molecule drug, antibody, antibody mimetic or functional equivalent or fragment thereof), whereby the active agent has an immunomodulatory function. In an embodiment, the immunomodulatory agent is any one or more of epacadostat, rapamycin, doxorubicin, valproic acid, mitoxantrone, vorinostat, cyclophosphamide, irinotecan, cisplatin, methotrexate, tacrolimus, an anti-CTLA-4 antibody or an anti-PD-1 antibody.

The skilled person will be well aware of other immunomodulatory agents encompassed within the above. Notably, the term “immunomodulatory agent”, as used herein, does not encompass compounds or compositions that function to enhance the immunogenicity of an antigen by prolonging the exposure of the antigen to immune cells (i.e. by a delivery platform, such as Freund'™ complete or incomplete adjuvant, Montanide™ ISA, or other oil-based carriers). Rather, the term “immunomodulatory agent”, as used herein, refers to a compound or composition that can be specifically delivered to the lymph nodes or lymphoid cells in a lymphatic tissue.

The amount of any specific immunomodulatory agent as described herein may depend on the type of agent (e.g. small molecule drug, antibody, etc.). One skilled in the art can readily determine the amount of immunomodulatory agent needed in a particular application by empirical testing.

Lipid-Based Structures

The compositions of the invention comprise one or more lipid-based structures. As used herein, the term “lipid-based structure” refers to any structure formed by one or more lipids.

The term “lipid” has its common meaning in the art in that it is any organic substance or compound that is soluble in nonpolar solvents, but generally insoluble in polar solvents (e.g. water). Lipids are a diverse group of compounds including, without limitation, fats, waxes, sterols, fat-soluble vitamins, monoglycerides, diglycerides, triglycerides and phospholipids. In an embodiment, the lipids of the lipid-based structures described herein are membrane-forming lipids. By “membrane-forming lipids” it is meant that the lipids, alone or together with other lipids and/or stabilizing molecules, are capable of forming a lipid membrane. The lipid membranes may form closed lipid vesicles or any other structure, such as for example lipid sheets. The lipid-based structures herein may comprise a single type of lipid or two or more different types of lipids.

In an embodiment, the lipid or lipids of the lipid-based structures are amphiphilic lipids, meaning that they possess both hydrophilic and hydrophobic (lipophilic) properties.

Although any lipid as defined above may be used, particularly suitable lipids may include those with at least one fatty acid chain containing at least 4 carbons, and typically about 4 to 28 carbons. The fatty acid chain may contain any number of saturated and/or unsaturated bonds. The lipid may be a natural lipid or a synthetic lipid. Non-limiting examples of lipids may include phospholipids, sphingolipids, sphingomyelin, cerobrocides, gangliosides, ether lipids, sterols, cardiolipin, cationic lipids and lipids modified with poly (ethylene glycol) and other polymers. Synthetic lipids may include, without limitation, the following fatty acid constituents: lauroyl, myristoyl, palmitoyl, stearoyl, arachidoyl, oleoyl, linoleoyl, erucoyl, or combinations of these fatty acids.

In an embodiment, the lipid is a phospholipid or a mixture of phospholipids. Broadly defined, a “phospholipid” is a member of a group of lipid compounds that yield on hydrolysis phosphoric acid, an alcohol, fatty acid, and nitrogenous base.

Phospholipids that may be used include for example, and without limitation, those with at least one head group selected from the group consisting of phosphoglycerol, phosphoethanolamine, phosphoserine, phosphocholine (e.g. DOPC; 1,2-Dioleoyl-sn-glycero-3-phosphocholine) and phosphoinositol. In an embodiment, the phospholipid may be phosphatidylcholine or a mixture of lipids comprising phosphatidylcholine. In an embodiment, the lipid may be DOPC (Lipoid GmbH, Germany) or Lipoid 5100 lecithin. In some embodiments, a mixture of DOPC and unesterified cholesterol may be used. In other embodiments, a mixture of Lipoid 5100 lecithin and unesterified cholesterol may be used.

In an embodiment, the lipid-based structures comprise a synthetic lipid. In an embodiment, the lipid-based structures comprise synthetic DOPC. In another embodiment, the lipid-based structures comprise synthetic DOPC and cholesterol.

When cholesterol is used, the cholesterol may be used in any amount sufficient to stabilize the lipids in the lipid membrane. In an embodiment, the cholesterol may be used in an amount equivalent to about 10% of the weight of phospholipid (e.g. in a DOPC:cholesterol ratio of 10:1 w/w). The cholesterol may stabilize the formation of phospholipid vesicle particles. If a compound other than cholesterol is used, one skilled in the art can readily determine the amount needed.

In an embodiment, the compositions disclosed herein comprise about 120 mg/ml of DOPC and about 12 mg/ml of cholesterol.

Another common phospholipid is sphingomyelin. Sphingomyelin contains sphingosine, an amino alcohol with a long unsaturated hydrocarbon chain. A fatty acyl side chain is linked to the amino group of sphingosine by an amide bond, to form ceramide. The hydroxyl group of sphingosine is esterified to phosphocholine. Like phosphoglycerides, sphingomyelin is amphipathic.

Lecithin, which also may be used, is a natural mixture of phospholipids typically derived from chicken eggs, sheep's wool, soybean and other vegetable sources.

All of these and other phospholipids may be used in the practice of the invention. Phospholipids can be purchased, for example, from Avanti lipids (Alabastar, Ala., USA), Lipoid LLC (Newark, N.J., USA) and Lipoid GmbH (Germany), among various other suppliers.

There are various lipid-based structures which may form, and the compositions disclosed herein may comprise a single type of lipid-based structure or comprise a mixture of different types of lipid-based structures.

In an embodiment, the lipid-based structures may be closed vesicular structures. They are typically spherical or substantially spherical in shape, but other shapes and conformations may be formed and are not excluded. By “substantially spherical” it is meant that the lipid-based structures are close to spherical, but may not be a perfect sphere. Other shapes of the closed vesicular structures include, without limitation, oval, oblong, square, rectangular, triangular, cuboid, crescent, diamond, cylinder or hemisphere shapes. Any regular or irregular shape may be formed. Exemplary embodiments of closed vesicular structures include, without limitation, single layer vesicular structures (e.g. micelles or reverse micelles) and bilayer vesicular structures (e.g. unilamellar or multilamellar vesicles), or various combinations thereof.

By “single layer” it is meant that the lipids do not form a bilayer, but rather remain in a layer with the hydrophobic part oriented on one side and the hydrophilic part oriented on the opposite side. By “bilayer” it is meant that the lipids form a two-layered sheet, such as with the hydrophobic part of each layer internally oriented toward the center of the bilayer with the hydrophilic part externally oriented. Alternatively, the opposite configuration is also possible, i.e. with the hydrophilic part of each layer internally oriented toward the center of the bilayer with the hydrophobic part externally oriented. The term “multilayer” is meant to encompass any combination of single and bilayer structures. The form adopted may depend upon the specific lipid that is used, and whether the composition is or is not water-free.

The closed vesicular structures may be formed from single layer lipid membranes, bilayer lipid membranes and/or multilayer lipid membranes. The lipid membranes are predominantly comprised of and formed by lipids, but may also comprise additional components. For example, and without limitation, the lipid membrane may include stabilizing molecules to aid in maintaining the integrity of the structure. Any available stabilizing molecule may be used.

In an embodiment, the lipid-based structure is a bilayer vesicular structure, such as for example, a liposome. Liposomes are completely closed lipid bilayer membranes. Liposomes may be unilamellar vesicles (possessing a single bilayer membrane), multilamellar vesicles (characterized by multimembrane bilayers whereby each bilayer may or may not be separated from the next by an aqueous layer) or multivesicular vesicles (possessing one or more vesicles within a vesicle). A general discussion of liposomes can be found in Gregoriadis 1990; and Frezard 1999. In an embodiment, the lipid-based structures are liposomes when the compositions herein are not water-free.

In an embodiment, the one or more lipid-based structures are comprised of a single layer lipid assembly. There are various types of these lipid-based structures which may form, and the compositions disclosed herein may comprise a single type of lipid-based structure having a single layer lipid assembly or comprise a mixture of different such lipid-based structures.

In an embodiment, the lipid-based structures herein have a single layer lipid assembly when the compositions herein are water-free.

In an embodiment, the lipid-based structure having a single layer lipid assembly partially or completely surrounds the active agent and/or immunomodulatory agent. As an example, the lipid-based structure may be a closed vesicular structure surrounding the active agent and/or immunomodulatory agent. In an embodiment, the hydrophobic part of the lipids in the vesicular structure is oriented outwards toward the hydrophobic carrier.

As another example, the one or more lipid-based structures having a single layer lipid assembly may comprise aggregates of lipids with the hydrophobic part of the lipids oriented outwards toward the hydrophobic carrier and the hydrophilic part of the lipids aggregating as a core. These structures do not necessarily form a continuous lipid layer membrane. In an embodiment, they are an aggregate of monomeric lipids.

In an embodiment, the one or more lipid-based structures having a single layer lipid assembly comprise reverse micelles. A typical micelle in aqueous solution forms an aggregate with the hydrophilic parts in contact with the surrounding aqueous solution, sequestering the hydrophobic parts in the micelle center. In contrast, in a hydrophobic carrier, an inverse/reverse micelle forms with the hydrophobic parts in contact with the surrounding hydrophobic solution, sequestering the hydrophilic parts in the micelle center. A spherical reverse micelle can package an active agent and/or immunomodulatory agent with hydrophilic affinity within its core (i.e. internal environment).

Without limitation, the size of the lipid-based structures having a single layer lipid assembly is in the range of from 2 nm (20 A) to 20 nm (200 A) in diameter. In an embodiment, the size of the lipid-based structures having a single layer lipid assembly is between about 2 nm to about 10 nm in diameter. In an embodiment, the size of the lipid-based structures having a single layer lipid assembly is about 2 nm, 3 nm, 4 nm, 5 nm, 6 nm, about 7 nm, about 8 nm, about 9 nm, or about 10 nm in diameter. In an embodiment, the maximum diameter of the lipid-based structures is about 4 nm or about 6 nm. In an embodiment, the lipid-based structures of these sizes are reverse micelles.

In an embodiment, one or more of the active agents and/or immunomodulatory agents are inside the lipid-based structures after solubilization in the hydrophobic carrier. By “inside the lipid-based structure” it is meant that the active agent and/or immunomodulatory agent is substantially surrounded by the lipids such that the hydrophilic components of the active agent and/or immunomodulatory agent are not exposed to the hydrophobic carrier. In an embodiment, the active agent and/or immunomodulatory agent inside the lipid-based structure is predominantly hydrophilic.

In an embodiment, one or more of the active agents and/or immunomodulatory agents are outside the lipid-based structures after solubilization in the hydrophobic carrier. By “outside the lipid-based structure”, it is meant that the active agent and/or immunomodulatory agent is not sequestered within the environment internal to the lipid membrane or assembly. In an embodiment, the active agent and/or immunomodulatory agent outside the lipid-based structure is predominantly hydrophobic.

Hydrophobic Carrier

The compositions comprise a hydrophobic carrier.

In an embodiment of a hydrophobic carrier, the carrier may comprise a continuous hydrophobic phase and discontinuous aqueous phase (e.g. an emulsion, such as for example a water-in-oil emulsion). In an embodiment of the hydrophobic carrier, the carrier comprises a continuous hydrophobic phase without a discontinuous phase (e.g. water-free).

The hydrophobic carrier may be an essentially pure hydrophobic substance or a mixture of hydrophobic substances. Hydrophobic substances that are useful in the methods and compositions described herein are those that are pharmaceutically acceptable. The carrier is typically a liquid at room temperature (e.g. about 18-25° C.), but certain hydrophobic substances that are not liquids at room temperature may be liquefied, for example by warming, and may also be useful.

Oil or a mixture of oils is a particularly suitable carrier for use in the methods and compositions disclosed herein. Oils should be pharmaceutically acceptable. Suitable oils include, for example, mineral oils (especially light or low viscosity mineral oil such as Drakeol® 6VR), vegetable oils (e.g., soybean oil), nut oils (e.g., peanut oil), or mixtures thereof. Thus, in an embodiment the hydrophobic carrier is a hydrophobic substance such as vegetable oil, nut oil or mineral oil. Animal fats and artificial hydrophobic polymeric materials, particularly those that are liquid at atmospheric temperature or that can be liquefied relatively easily, may also be used.

In some embodiments, the hydrophobic carrier may be, or comprise, Incomplete Freund's Adjuvant (IFA), a mineral oil-based model hydrophobic carrier. In another embodiment, the hydrophobic carrier may be, or comprise, a mannide oleate in mineral oil solution, such as that commercially available as Montanide® ISA 51 (SEPPIC, France).

In an embodiment, the hydrophobic carrier is mineral oil or a mannide oleate in mineral oil solution.

In an embodiment, the hydrophobic carrier is Montanide® ISA 51.

In an embodiment, the hydrophobic carrier is MS80 oil, which is a mixture of mineral oil (Sigma Aldrich) and Span80 (Fluka). The components can be purchased separately and mixed prior to use.

In an embodiment, the hydrophobic carrier is water-free and is used to prepare a composition that is water-free as described elsewhere herein. Again, as used herein, “water-free” means completely or substantially free of water. In an embodiment, the hydrophobic carrier is completely free of water.

Methods for Preparing the Compositions

The compositions may be prepared by known methods in the art having regard to the present disclosure. Exemplary embodiments for preparing the compositions disclosed herein are described below, including in the examples, without limitation.

As used in this section, the term “active agent and/or immunomodulatory agent” is used generally to describe any of the active agents and/or immunomodulatory agents as described and defined herein. The term “active agent and/or immunomodulatory agent” encompasses both the singular form “active agent and/or immunomodulatory agent” and the plural “active agents and/or immunomodulatory agents”. If multiple active agents and/or immunomodulatory agents are included in the composition, it is not necessary that they all be introduced into the composition in the same way.

In an embodiment for preparing the composition, a lipid preparation is prepared by dissolving or hydrating lipids, or a lipid-mixture, in a suitable solvent with gently shaking. The active agent and/or immunomodulatory agent may then be added to the lipid preparation, either directly (e.g. adding dry active agent and/or immunomodulatory agent) or by first preparing a stock of the active agent and/or immunomodulatory agent dissolved in a suitable solvent. Typically, the active agent and/or immunomodulatory agent is added to, or combined with, the lipid preparation with gently shaking. The active agent and/or immunomodulatory agent/lipid preparation is then dried to form a dry cake, and the dry cake is resuspended in a hydrophobic carrier. The step of drying may be performed by various means known in the art, such as by freeze-drying, lyophilization, rotary evaporation, evaporation under pressure, etc. Low heat drying that does not compromise the integrity of the components can also be used.

The “suitable solvent” is one that is capable of dissolving the respective component (e.g. lipids, active agent/immunomodulatory agent, or both), and can be determined by the skilled person.

In respect of the active agents and/or immunomodulatory agents, in an embodiment the suitable solvent is sodium phosphate buffer or sodium acetate buffer. In another embodiment, DMSO or water may be used. The skilled person can determine other suitable solvents depending on the active agent and/or immunomodulatory agent to be used.

In respect of the lipids, in an embodiment the suitable solvent is a polar protic solvent such as an alcohol (e.g. tert-butanol, n-butanol, isopropanol, n-propanol, ethanol or methanol), water, acetate buffer, phosphate buffer, formic acid or chloroform. In an embodiment, the suitable solvent is 40% tertiary-butanol. The skilled person can determine other suitable solvents depending on the lipids to be used.

In a particular embodiment to prepare the compositions, a lipid-mixture containing DOPC and cholesterol in a 10:1 ratio (w:w) (Lipoid GmBH, Germany) can be dissolved in 40% tertiary-butanol by shaking at 300 RPM at room temperature until dissolved. An active agent/immunomodulatory agent stock can be prepared in DMSO and diluted with 40% tertiary-butanol prior to mixing with the dissolved lipid-mixture. Active agent/immunomodulatory agent stock can then be added to the dissolved lipid-mixture with shaking at 300 RPM for about 5 minutes. The preparation can then be freeze-dried for storage and later reconstitution. Optionally, the preparation can be freeze-dried with cryoprotectants/bulking agents. Cryoprotectants/bulking agents that can be used include, but are not limited to sugars/polysaccharides such as trehalose, sucrose, mannitol, sorbitol, lactose, maltose, raffinose, maltodextrin, pullulan, inulin, ficoll, carboxymethylcellulose, and hydroxyethyl starch; amino acids such as arginine, histidine, phenylalanine, leucine, and isoleucine; bovine serum albumin; buffer salts such as sodium acetate, sodium phosphate, Tris HC1, HEPES, sodium carbonate, sodium citrate, Tris acetate; and polymers such as poly vinyl pyrrolidone, poly vinyl alcohol, hydroxypropyl-β-cyclodextrin, polyacrylamide, and Carbopol®. The freeze-dried cake can then be reconstituted in Montanide® ISA 51 VG (SEPPIC, France) to obtain a clear solution. Typically, the freeze-dried cake is stored (e.g. at −20° C.) until the time of administration, when the freeze-dried cake is reconstituted in the hydrophobic carrier.

In another embodiment, to prepare the compositions the active agent and/or immunomodulatory agent is dissolved in sodium phosphate buffer with 5100 lipids and cholesterol (Lipoid, Germany). These components are then lyophilized to form a dry cake. Just prior to injection, the dry cake is resuspended in ISA51 VG oil (SEPPIC, France) to prepare a water-free oil-based composition.

In another embodiment, to prepare the compositions the active agent and/or immunomodulatory agent is dissolved in sodium phosphate buffer with DOPC and cholesterol (Lipoid, Germany). These components are then lyophilized to form a dry cake. Just prior to injection, the dry cake is resuspended in ISA51 VG oil (SEPPIC, France) to prepare a water-free oil-based composition.

In another embodiment, to prepare the compositions the active agent and/or immunomodulatory agent is mixed with lipid/cholesterol nanoparticles (size ≤110 nm) in sodium phosphate buffer (100 mM, pH 6.0). The lipid may be DOPC. The components are then lyophilized to form a dry cake. Just prior to injection, the dry cake is resuspended in ISA51 VG oil (SEPPIC, France) to prepare a water-free oil-based composition.

In some embodiments, it may be appropriate to include an emulsifier in the hydrophobic carrier to assist in stabilizing the components of the dry cake when they are resuspended in the hydrophobic carrier. The emulsifier is provided in an amount sufficient to resuspend the dry mixture of active agent and/or immunomodulatory agent and lipids in the hydrophobic carrier and maintain the active agent and/or immunomodulatory agent and lipids in a dissolved state in the hydrophobic carrier. For example, the emulsifier may be present at about 5% to about 15% weight/weight or weight/volume of the hydrophobic carrier.

Stabilizers such as sugars, anti-oxidants, or preservatives that maintain the biological activity or improve chemical stability to prolong the shelf life of any of the components, may be added to the compositions.

In an embodiment, methods for preparing the compositions herein may include those disclosed in WO 2009/043165, as appropriate in the context of the present disclosure. In such instances, the active agents and/or immunomodulatory agents as described herein would be incorporated into the compositions in similar fashion as described for antigens in WO 2009/043165.

In an embodiment, methods for preparing the compositions herein may include those disclosed in PCT/CA2017/051335 and PCT/CA2017/051335 involving the use of sized lipid vesicle particles. In such instances, the active agents and/or immunomodulatory agents as described herein would be incorporated into the compositions in similar fashion as described for therapeutic agents in PCT/CA2017/051335 and PCT/CA2017/051335.

### Embodiments

Particular embodiments of the invention include, without limitation, the following:

(1) A method for targeted delivery of an active agent to lymph nodes or lymphoid cells in a lymphatic tissue, said method comprising administering to a subject in need thereof a composition comprising: a) an active agent, wherein the active agent is a small molecule drug; an antibody, an antibody mimetic, or a functional equivalent or functional fragment of any one thereof; or a mixture thereof, b) one or more lipid-based structures, and c) a hydrophobic carrier.

(2) The method of paragraph (1), wherein the composition is water-free.

(3) The method of paragraph (1) or (2), wherein the lymphatic tissue is a lymph node.

(4) The method of paragraph (1) or (2), wherein the lymphatic tissue is a spleen, thymus or mucosal-associated lymphoid tissue.

(5) The method of any one of paragraphs (1) to (3), wherein the active agent is delivered to immune cells in the lymph nodes.

(6) The method of paragraph (5), wherein the immune cells are T-lymphocytes, B-lymphocytes, or both.

(7) The method of any one of paragraphs (1) to (6), wherein the active agent is delivered to the lymph nodes or lymphoid cells in a lymphatic tissue by dendritic cells or macrophages.

(8) The method of paragraph (7), wherein the active agent is delivered to the dendritic cells or macrophages at or near the site of administration of the composition.

(9) The method of any one of paragraphs (1) to (8), wherein the active agent does not directly bind a major histocompatibility complex (MHC) class I protein, an MHC class II protein, or both.

(10) The method of paragraph (9), wherein the active agent does not directly bind an MHC class I protein.

(11) The method of any one of paragraphs (1) to (10), wherein the active agent is delivered intact to the lymph nodes or lymphoid cells.

(12) The method of any one of paragraphs (1) to (11), wherein the active agent binds a checkpoint receptor on the surface of T-lymphocytes.

(13) The method of any one of paragraphs (1) to (12), wherein the active agent is an immunomodulatory agent.

(14) The method of any one of paragraphs (1) to (11) wherein the active agent is a shuttle.

(15) The method of any one of paragraphs (1) to (14) wherein the active agent is a small molecule drug.

(16) The method of any one of paragraphs (1) to (15) wherein the small molecule drug has a molecular weight of about 2000 Daltons or less than 2000 Daltons.

(17) The method of any one of paragraphs (1) to (15) wherein the small molecule drug has a molecular weight of about 900 Daltons or less than 900 Daltons.

(18) The method of any one of paragraphs (15) to (17), wherein the small molecule drug is a cytotoxic agent, an anti-tumor agent, a chemotherapeutic agent, an anti-neoplastic agent, an antiviral agent, an antibacterial agent, an anti-inflammatory agent, an immunomodulatory agent, an immune response checkpoint agent, a biological response modifier, a prodrug, a cytokine, a chemokine, a vitamin, a steroid, a ligand, an analgesic, a radiopharmaceutical, a radioisotope or a dye for visual detection.

(19) The method of paragraph (15) wherein the active agent is epacadostat, rapamycin, doxorubicin, valproic acid, mitoxantrone, vorinostat, cyclophosphamide, irinotecan, cisplatin, methotrexate, tacrolimus or a pharmaceutically acceptable salt of any one thereof

(20) The method of paragraph (19), wherein the active agent is epacadostat.

(21) The method of paragraph (19), wherein the active agent is cyclophosphamide.

(22) The method of paragraph (19), wherein the active agent is rapamycin.

(23) The method of any one of paragraphs (1) to (14), wherein the active agent is an antibody, an antibody mimetic, or a functional equivalent or functional fragment of any one thereof.

(24) The method of paragraph (23), wherein the active agent is an anti-CTLA-4 antibody.

(25) The method of paragraph (24), wherein the anti-CTLA-4 antibody is ipilimumab, tremelimumab, BN-13, UC10-4F10-11, 9D9 or 9H10.

(26) The method of paragraph (23), wherein the active agent is an anti-PD-1 antibody or an anti-PD-L1 antibody.

(27) The method of paragraph (26), wherein the anti-PD-1 antibody is pembrolizumab, nivolumab, pidilizumab, AMP-224, RMP1-4 or J43 and the anti-PD-L1 antibody is atezolizumab, avelumab, BMS-936559 or durvalumab.

(28) The method of paragraph (23), wherein the active agent is a peptide aptamer, an affimer, an affilin, an affibody, an affitin, an alphabody, an anticalin, an avimer, a DARPin, a fynomer, a Kunitz domain peptide, a monobody, a nanoCLAMP, an affinity reagent, a scaffold protein, a monoclonal antibody, a chimeric antibody, a humanized antibody, a F(ab′)₂, a F(ab)₂, a Fab′, a Fab, a Fab₂, a Fab3, a Fv, a scFv, a Dab, an evibody, a minibody, a diabody, a triabody, a tetrabody, or a single-domain antibody (nanobody).

(29) The method of any one of paragraphs (1) to (28), wherein the active agent exhibits systemic delivery in the subject when administered in a composition that does not include one or both of the components (b) and (c).

(30) A method for targeted delivery of an immunomodulatory agent to lymph nodes or lymphoid cells in a lymphatic tissue, said method comprising administering to a subject in need thereof a composition comprising: a) an immunomodulatory agent, b) one or more lipid-based structures, and c) a hydrophobic carrier.

(31) The method of paragraph (30), wherein the composition is water-free.

(32) The method of paragraph (30) or 31), wherein the lymphatic tissue is a lymph node.

(33) The method of paragraph (30) or (31), wherein the lymphatic tissue is a spleen, thymus or mucosal-associated lymphoid tissue.

(34) The method of any one of paragraphs (30) to (32), wherein the immunomodulatory agent is delivered to immune cells in the lymph nodes.

(35) The method of paragraph (34), wherein the immune cells are T-lymphocytes, B-lymphocytes, or both.

(36) The method of any one of paragraphs (30) to (35), wherein the immunomodulatory agent is delivered to the lymph nodes or lymphoid cells in a lymphatic tissue by dendritic cells or macrophages.

(37) The method of paragraph (36), wherein the active agent is delivered to the dendritic cells or macrophages at or near the site of administration of the composition.

(38) The method of any one of paragraphs (30) to (37), wherein the immunomodulatory agent is delivered intact to the lymph nodes or lymphoid cells.

(39) The method of any one of paragraphs (30) to (38), wherein the immunomodulatory agent binds a checkpoint receptor on the surface of T-lymphocytes.

(40) The method of any one of paragraphs (30) to (39), wherein the immunomodulatory agent upregulates, downregulates or reprograms the type of immune response that is activated by an antigen or immunogen.

(41) The method of any one of paragraphs (30) to (40), wherein the immunomodulatory agent is a small molecule drug; an antibody, an antibody mimetic, or a functional equivalent or functional fragment of any one thereof; or a mixture thereof

(42) The method of paragraph (41), wherein the immunomodulatory agent is epacadostat, rapamycin, doxorubicin, valproic acid, mitoxantrone, vorinostat, cyclophosphamide, irinotecan, cisplatin, methotrexate, tacrolimus, an anti-CTLA-4 antibody or an anti-PD-1 antibody.

(43) The method of any one of paragraphs (30) to (42), wherein the immunomodulatory agent exhibits systemic delivery in the subject when administered in a composition that does not include one or both of the components (b) and (c).

(44) The method of any one of paragraphs (1) to (43), wherein the one or more lipid-based structures have a single layer lipid assembly.

(45) The method of paragraph (44), wherein the one or more lipid-based structures having a single layer lipid assembly comprise aggregates of lipids with the hydrophobic part of the lipids oriented outwards toward the hydrophobic carrier and the hydrophilic part of the lipids aggregating as a core.

(46) The method of paragraph (45), wherein the one or more lipid-based structures having a single layer lipid assembly comprise reverse micelles.

(47) The method of any one of paragraphs (1) to (46), wherein the size of the lipid-based structures is between about 2 nm to about 10 nm in diameter.

(48) The method of any one of paragraphs (1) to (47), wherein the hydrophobic carrier is mineral oil or a mannide oleate in mineral oil solution.

(49) The method of any one of paragraphs (1) to (48), wherein the hydrophobic carrier is Montanide® ISA 51.

(50) The method of any one of paragraphs (1) to (49), wherein the administration is by injection.

(51) The method of paragraph (50), wherein the administration is by subcutaneous, intramuscular, or intraperitoneal injection.

(52) The method of any one of paragraphs (1) to (51), which is for modulating an immune response in a subject.

(53) The method of any one of paragraphs (1) to (51), which is for treating or preventing a disease or disorder in the subject.

(54) The method of paragraph (53), wherein the disease or disorder is an infectious disease or cancer.

(55) Use of a composition comprising: a) an active agent, wherein the active agent is a small molecule drug; an antibody, an antibody mimetic, or a functional equivalent or functional fragment of any one thereof; or a mixture thereof; b) one or more lipid-based structures; and c) a hydrophobic carrier, for targeting the active agent to lymph nodes or lymphoid cells in a lymphatic tissue in a subject.

(56) The use of paragraph (55), wherein the composition is water-free.

(57) The use of paragraph (55) or (56), wherein the lymphatic tissue is a lymph node.

(58) The use of paragraph (55) or (56), wherein the lymphatic tissue is a spleen, thymus or mucosal-associated lymphoid tissue.

(59) The use of any one of paragraphs (55) to (58), wherein the active agent is delivered to immune cells in the lymph nodes.

(60) The use of paragraph (59), wherein the immune cells are T-lymphocytes, B-lymphocytes, or both.

(61) The use of any one of paragraphs (55) to (60), wherein the active agent is delivered to the lymph nodes or lymphoid cells in a lymphatic tissue by dendritic cells or macrophages.

(62) The use of paragraph (61), wherein the active agent is delivered to the dendritic cells or macrophages at or near the site of administration of the composition.

(63) The use of any one of paragraphs (55) to (62), wherein the active agent does not directly bind a major histocompatibility complex (WIC) class I protein, an WIC class II protein, or both.

(64) The use of paragraph (63), wherein the active agent does not directly bind an WIC class I protein.

(65) The use of any one of paragraphs (55) to (64), wherein the active agent is delivered intact to the lymph nodes or lymphoid cells.

(66) The use of any one of paragraphs (55) to (65), wherein the active agent binds a checkpoint receptor on the surface of T-lymphocytes.

(67) The use of any one of paragraphs (55) to (66), wherein the active agent is an immunomodulatory agent.

(68) The use of any one of paragraphs (55) to (65) wherein the active agent is a shuttle.

(69) The use of any one of paragraphs (55) to (68) wherein the active agent is a small molecule drug.

(70) The use of any one of paragraphs (55) to (69) wherein the small molecule drug has a molecular weight of about 2000 Daltons or less than 2000 Daltons.

(71) The use of any one of paragraphs (55) to (69) wherein the small molecule drug has a molecular weight of about 900 Daltons or less than 900 Daltons.

(72) The use of paragraph (69) or (70), wherein the small molecule drug is a cytotoxic agent, an anti-tumor agent, a chemotherapeutic agent, an anti-neoplastic agent, an antiviral agent, an antibacterial agent, an anti-inflammatory agent, an immunomodulatory agent, an immune response checkpoint agent, a biological response modifier, a prodrug, a cytokine, a chemokine, a vitamin, a steroid, a ligand, an analgesic, a radiopharmaceutical, a radioisotope or a dye for visual detection.

(73) The use of paragraph (69) wherein the active agent is epacadostat, rapamycin, doxorubicin, valproic acid, mitoxantrone, vorinostat, cyclophosphamide, irinotecan, cisplatin, methotrexate, tacrolimus or a pharmaceutically acceptable salt of any one thereof.

(74) The use of paragraph (73), wherein the active agent is epacadostat.

(75) The use of paragraph (73), wherein the active agent is cyclophosphamide.

(76) The use of paragraph (73), wherein the active agent is rapamycin.

(77) The use of any one of paragraphs (55) to (69), wherein the active agent is an antibody, an antibody mimetic, or a functional equivalent or functional fragment of any one thereof.

(78) The use of paragraph (77), wherein the active agent is an anti-CTLA-4 antibody.

(79) The use of paragraph (78), wherein the anti-CTLA-4 antibody is ipilimumab, tremelimumab, BN-13, UC10-4F10-11, 9D9 or 9H10.

(80) The use of paragraph (77), wherein the active agent is an anti-PD-1 antibody or an anti-PD-L1 antibody.

(81) The use of paragraph (80), wherein the anti-PD-1 antibody is pembrolizumab, nivolumab, pidilizumab, AMP-224, RMP1-4 or J43 and the anti-PD-L1 antibody is atezolizumab, avelumab, BMS-936559 or durvalumab.

(82) The use of paragraph (77), wherein the active agent is a peptide aptamer, an affimer, an affilin, an affibody, an affitin, an alphabody, an anticalin, an avimer, a DARPin, a fynomer, a Kunitz domain peptide, a monobody, a nanoCLAMP, an affinity reagent, a scaffold protein, a monoclonal antibody, a chimeric antibody, a humanized antibody, a F(ab′)₂, a F(ab)₂, a Fab′, a Fab, a Fab₂, a Fab3, a Fv, a scFv, a Dab, an evibody, a minibody, a diabody, a triabody, a tetrabody, or a single-domain antibody (nanobody).

(83) The use of any one of paragraphs (55) to (82), wherein the active agent exhibits systemic delivery in the subject when administered in a composition that does not include one or both of the components (b) and (c).

(84) Use of a composition comprising: a) an immunomodulatory agent; b) one or more lipid-based structures; and c) a hydrophobic carrier, for targeting the immunomodulatory agent to lymph nodes or lymphoid cells in a lymphatic tissue in a subject.

(85) The use of paragraph (84), wherein the composition is water-free.

(86) The use of paragraph (84) or (85), wherein the lymphatic tissue is a lymph node.

(87) The use of paragraph (84) or (85), wherein the lymphatic tissue is a spleen, thymus or mucosal-associated lymphoid tissue.

(88) The use of any one of paragraphs (84) to (87), wherein the immunomodulatory agent is delivered to immune cells in the lymph nodes.

(89) The use of paragraph (88), wherein the immune cells are T-lymphocytes, B-lymphocytes, or both.

(90) The use of any one of paragraphs (84) to (89), wherein the immunomodulatory agent is delivered to the lymph nodes or lymphoid cells in a lymphatic tissue by dendritic cells or macrophages.

(91) The use of paragraph (90), wherein the active agent is delivered to the dendritic cells or macrophages at or near the site of administration of the composition.

(92) The use of any one of paragraphs (84) to (91), wherein the immunomodulatory agent is delivered intact to the lymph nodes or lymphoid cells.

(93) The use of any one of paragraphs (84) to (92), wherein the immunomodulatory agent binds a checkpoint receptor on the surface of T-lymphocytes.

(94) The use of any one of paragraphs (84) to (93), wherein the immunomodulatory agent upregulates, downregulates or reprograms the type of immune response that is activated by an antigen or immunogen.

(95) The use of any one of paragraphs (84) to (94), wherein the immunomodulatory agent is a small molecule drug; an antibody, an antibody mimetic, or a functional equivalent or functional fragment of any one thereof; or a mixture thereof.

(96) The use of paragraph (95), wherein the immunomodulatory agent is epacadostat, rapamycin, doxorubicin, valproic acid, mitoxantrone, vorinostat, cyclophosphamide, irinotecan, cisplatin, methotrexate, tacrolimus, an anti-CTLA-4 antibody or an anti-PD-1 antibody.

(97) The use of any one of paragraphs (84) to (96), wherein the immunomodulatory agent exhibits systemic delivery in the subject when administered in a composition that does not include one or both of the components (b) and (c).

(98) The use of any one of paragraphs (55) to (66), wherein the one or more lipid-based structures have a single layer lipid assembly.

(99) The use of paragraph (98), wherein the one or more lipid-based structures having a single layer lipid assembly comprise aggregates of lipids with the hydrophobic part of the lipids oriented outwards toward the hydrophobic carrier and the hydrophilic part of the lipids aggregating as a core.

(100) The use of paragraph (99), wherein the one or more lipid-based structures having a single layer lipid assembly comprise reverse micelles.

(101) The use of any one of paragraphs (55) to (100), wherein the size of the lipid-based structures is between about 2 nm to about 10 nm in diameter.

(102) The use of any one of paragraphs (55) to (101), wherein the hydrophobic carrier is mineral oil or a mannide oleate in mineral oil solution.

(103) The use of any one of paragraphs (55) to (102), wherein the hydrophobic carrier is Montanide® ISA 51.

(104) The use of any one of paragraphs (55) to (103), wherein the administration is by injection.

(105) The use of paragraph (104), wherein the administration is by subcutaneous, intramuscular, or intraperitoneal injection.

(106) The use of any one of paragraphs (55) to (105), which is for modulating an immune response in a subject.

(107) The use of any one of paragraphs (55) to (105), which is for treating or preventing a disease or disorder in the subject.

(108) The use of paragraph (107), wherein the disease or disorder is an infectious disease or cancer.

The invention is further illustrated by the following non-limiting examples.

### EXAMPLES

The invention will now be described by way of non-limiting examples having regard to the appended drawings.

### Example 1

Pathogen free, C57BL/6 mice, 6-8 weeks of age, were purchased from Charles River Laboratories (St. Constant, PQ) and housed according to institutional guidelines with water and food ad libitum under filter controlled air circulation.

A first composition was prepared with cyclophosphamide (CPA; Sigma-Aldrich, St. Louis, Mo.); a second composition was prepared with R9F-PADRE fusion peptide (FP) which contains the HPV16E7₄₉₋₅₇ peptide antigen (R9F; RAHYNIVTF) conjugated to a universal T-helper peptide PADRE (AKXVAAWTLKAA; wherein X is cyclohexylalanyl), and a DNA based poly I:C polynucleotide adjuvant (a 26-mer sequence of (IC)₁₃, i.e. ICICICICICICICICICICICICIC); and a third composition was prepared with cyclophosphamide, FP and the DNA based polyI:C polynucleotide.

Mice were administered the compositions and lymph node cell counts were analyzed, as described below.

Preparation of Compositions

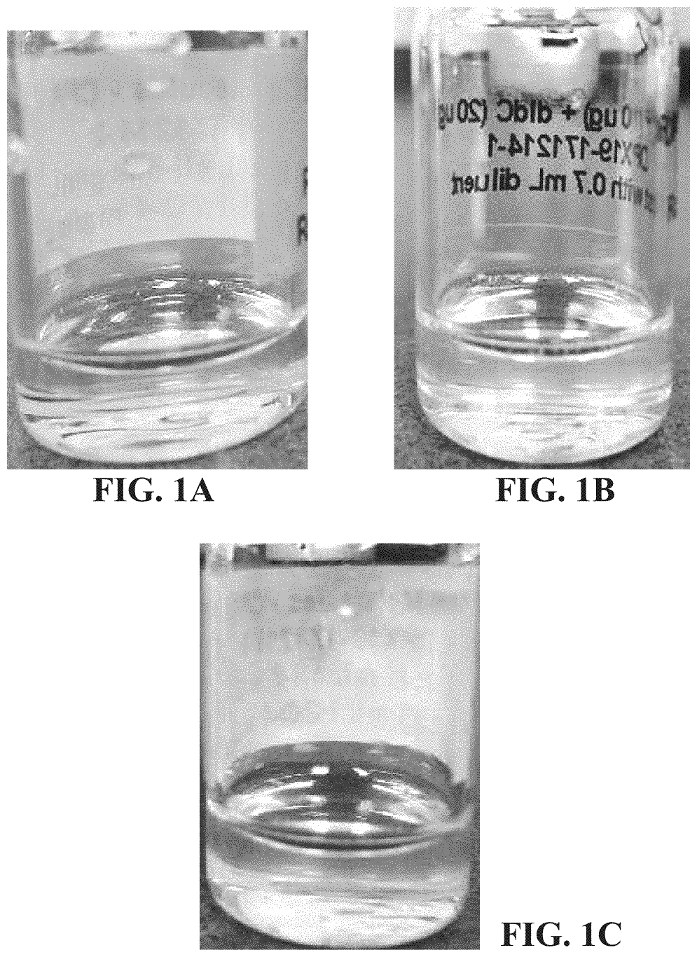

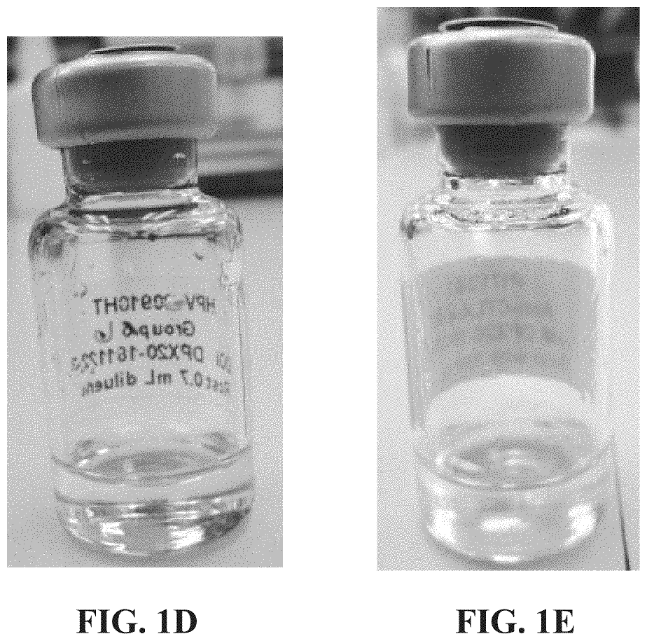

CPA Composition: CPA compositions were prepared by adding CPA drug to a lipid-mixture solution, mixing well and freeze-drying. Briefly, a lipid-mixture (132 mg/mL) containing DOPC and cholesterol in a 10:1 ratio (w:w) (Lipoid GmBH, Germany) was dissolved in 40% tertiary-butanol by shaking well at 300 RPM at room temperature for 1 hour or until dissolved. Next, a CPA drug stock was prepared at 250 mg/mL in DMSO and diluted with 40% tertiary-butanol to obtain a 125 mg/mL stock. Aliquots of 0.5 mL of the prepared lipid-mixture solution were placed into two vials, CPA drug stock was then added (3.2 μL in the first vial and 32 μL in the second vial) to obtain preparations with lower and higher CPA concentrations (0.4 mg/mL and 4.0 mg/mL), and mixed well by shaking at 300 RPM for 5 minutes. Each preparation was then Q.S to 1.0 mL with 40% tertiary-butanol and freeze-dried. The freeze-dried cake was then reconstituted in 0.45 mL of Montanide® ISA 51 VG (SEPPIC,France) to obtain a clear solution (FIG. 1; Panel A).

FP/DNA based PolyI:C Composition: The FP/DNA based polyI:C Composition was prepared by adding FP and DNA based polyI:C polynucleotide adjuvant stock to a lipid-mixture solution, mixing well and freeze-drying. Briefly, a lipid-mixture (132 mg/mL) containing DOPC and cholesterol in a 10:1 ratio (w:w) (Lipoid GmBH, Germany) was dissolved in 40% tertiary-butanol by shaking well at 300 RPM at room temperature for 1 hour or until dissolved. Next, FP stock (10 mg/mL) was prepared in DMSO and DNA based polyI:C polynucleotide adjuvant stock (10 mg/mL) was prepared in sterile water. To a 0.5 mL aliquot of lipid-mixture solution, 10 μL of FP stock was added to obtain 0.1 mg/mL final fill concentration, shaken well at 300 RPM for 5 minutes. To the formed FP-lipid-mixture solution, 20 μL of DNA based polyI:C polynucleotide adjuvant stock was added to obtain 0.2 mg/mL final fill concentration, shaken well at 300 RPM for 5 minutes. Q.S to 1.0 mL with 40% tertiary-butanol and freeze-dried. The freeze-dried cake was then reconstituted in 0.45 mL of Montanide® ISA 51 VG (SEPPIC, France) to obtain a clear to slight hazy solution (FIG. 1; Panel B).

CPA/FP/DNA based PolyI:C Composition: The CPA/FP/DNA based polyI:C polynucleotide composition was prepared by adding FP, CPA and DNA based polyI:C polynucleotide adjuvant stocks to a lipid-mixture solution, mixing well and freeze-drying. Briefly, a lipid-mixture (132 mg/mL) containing DOPC and cholesterol in a 10:1 ratio (w:w) (Lipoid GmBH, Germany) was dissolved in 40% tertiary-butanol by shaking well at 300 RPM at room temperature for 1 hour or until dissolved. Next, FP stock (10 mg/mL) was prepared in DMSO and DNA based polyI:C polynucleotide adjuvant stock (10 mg/mL) was prepared in sterile water. CPA drug stock was prepared at 250 mg/mL in DMSO and diluted with 40% tertiary-butanol to obtain a 125 mg/mL stock. To two 0.5 mL aliquots of lipid-mixture solution, 10 μL of FP stock was added to obtain 0.1 mg/mL fill concentration, shaken well at 300 RPM for 5 minutes. To the formed FP-lipid-mixture solution, CPA drug stock was then added (3.2 μL in the first vial and 32 μL in the second vial) to obtain preparations with lower and higher CPA concentrations (0.4 mg/mL and 4.0 mg/mL), and mixed well by shaking at 300 RPM for 5 minutes. 20 μL of DNA based polyI:C polynucleotide adjuvant stock was then added to each vial to obtain 0.2 mg/mL fill concentration, and shaken well at 300 RPM for 5 minutes. Each preparation was then Q.S to 1.0 mL with 40% tertiary-butanol and freeze-dried. The freeze-dried cake was then reconstituted in 0.45 mL of Montanide® ISA 51 VG (SEPPIC, France) to obtain a clear to slight hazy solution (FIG. 1; Panel C).

HPLC Analysis of Compositions

The HPLC instrument conditions and the gradient profile tested are shown the tables below. Samples were quantified using a 5-point calibration curve in the range of 5 μg/mL to 125 μg/mL CPA.

HPLC Instrument Conditions

| HPLC Model | Agilent 1100 Series or 1200 Series |
|---|---|
| HPLC Column | Phenomenex Gemini-NX C18, 110 Å, |
| | 250 × 4.60 mm with guard cartridge |
| Mobile Phase A | 70% Water/30% Acetonitrile |
| Mobile Phase B | Methanol (wash) |
| Flow Rate | 1.0 mL/min |
| Column Temperature | 60° C. |
| Injection Volume | 50 μL |
| Detector | UV |
| Detection (nm) | 197 |
| Run Time (minutes) | 25 |
| Post Time (minutes) | 15 |

Gradient Profile

| Step | Time | | |
|---|---|---|---|
| Number | (minutes) | % MPA | % MPB |
| 1 | 0.00 | 100 | 0 |
| 2 | 10.00 | 100 | 0 |
| 3 | 10.01 | 0 | 100 |
| 4 | 25.00 | 0 | 100 |

Experimental Conditions:

Solubilize the CPA lyophilisate or the CPA/FP/DNA based PolyI:C lyophilisate with Ultrapure lab water to give 1 mg/mL CPA. Add 3-4 glass beads and vortex vigorously for 1 minute to assure complete homogenization. Transfer 75 mg of solution to a 5 mL volumetric flask and dilute to the mark with Mobile Phase A (theoretical concentration of CPA is 15 μg/mL).

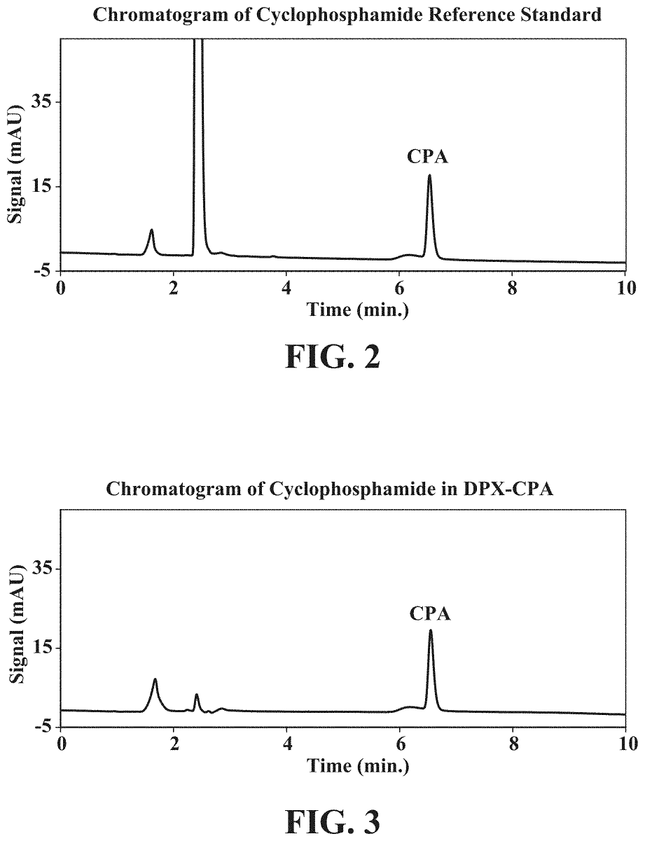

An HPLC chromatogram of a reference standard containing 15 μg/mL CPA is shown in FIG. 2.

A sample of the CPA composition, prepared as above, was characterized quantitatively using the HPLC method. The HPLC chromatogram showing CPA after freeze-drying is shown in FIG. 3. The calculated recovery of CPA from the dried preparation was 102%.

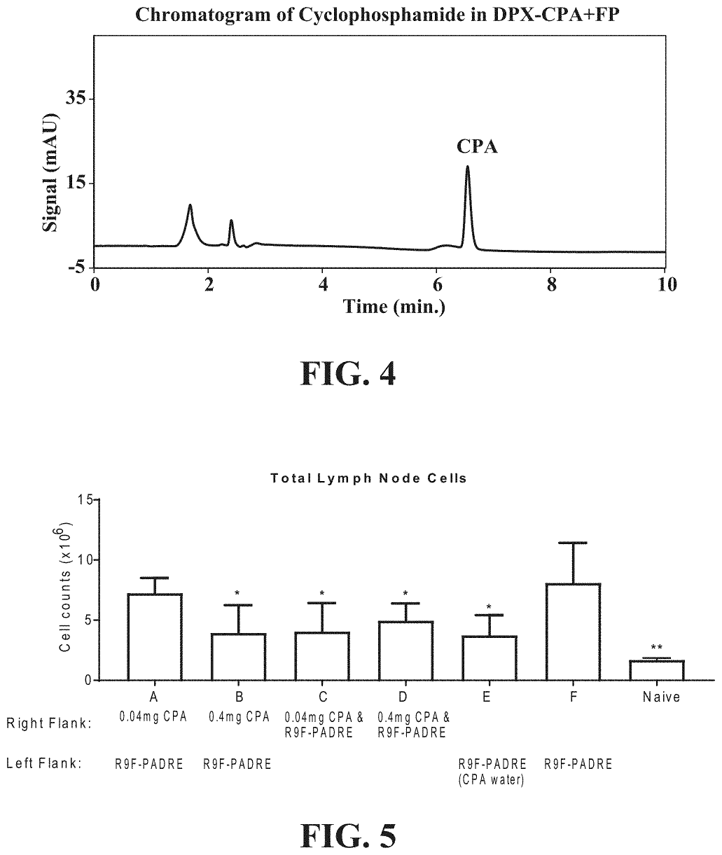

A sample of the CPA/FP/DNA based PolyI:C composition, prepared as above, was characterized quantitatively using the HPLC method. The HPLC chromatogram showing CPA after freeze-drying is shown in FIG. 4. The calculated recovery of cyclophosphamide from the dried preparation was 100%.

In Vivo Studies

Mice in group A (n=5) were injected in the right flank with 50 microliters of the CPA composition containing 0.04 milligrams of CPA and injected in the left flank with 50 microliters of the FP/DNA based PolyI:C polynucleotide composition containing 10 micrograms of FP and 20 micrograms of DNA based polyl:C polynucleotide adjuvant.

Mice in group B (n=5) were injected in the right flank with 50 microliters of the CPA composition containing 0.4 milligrams of CPA and injected in the left flank with 50 microliters of the FP/DNA based PolyI:C polynucleotide composition containing 10 micrograms of FP and 20 micrograms of DNA based polyl:C polynucleotide adjuvant.

Mice in group C (n=5) were injected in the right flank with 50 microliters of the CPA/FP/PolyI:C composition containing 0.04 milligrams of CPA and 10 micrograms of FP and 20 micrograms of DNA based polyI:C polynucleotide adjuvant.

Mice in group D (n=5) were injected in the right flank with 50 microliters of the CPA/FP/PolyI:C composition containing 0.4 milligrams of CPA and 10 micrograms of FP and 20 micrograms of DNA based polyI:C polynucleotide adjuvant.

Mice in group E (n=5) were injected in the left flank with 50 microliters of the FP/PolyI:C composition. For seven days prior to injection of the FP/DNA based PolyI:C composition, these mice also received treatment with 20 mg/kg/day CPA provided in the drinking water, which corresponds to approximately 0.4 milligrams per day for seven days.

Mice in group F (n=5) were injected in the left flank with 50 microliters of the FP/PolyI:C composition.

All mice were terminated 8 days following injections. Inguinal lymph nodes from the side of the mouse receiving the FP antigen were collected and processed into a single cell suspension. Total lymph node counts are shown in FIG. 5.

The lymph nodes of mice in Group F, which were injected with the FP/DNA based PolyI:C composition only, had more cells than the naïve mice, indicative of active immune responses. Mice in group E had significantly lower cell counts compared to group F, as expected when mice are treated with CPA. Mice in groups B, C, and D also had significantly lower cell counts compared to group F. Mice in groups A had lower cell counts than group F, but they were not significant.

This data indicates that CPA delivered in a composition of the invention, comprising lipid-based structures and a hydrophobic carrier, induces a reduction in lymph node cells in mice vaccinated with FP antigen similar to that observed when CPA is delivered through drinking water. However, the equivalent result with the CPA compositions of the invention was achieved with only a single administration, whereas oral CPA required administration over a period of one week with a significantly greater total quantity of CPA. The results evidence the efficiency of the compositions of the invention in providing targeted delivery of the CPA to lymph nodes.

### Example 2

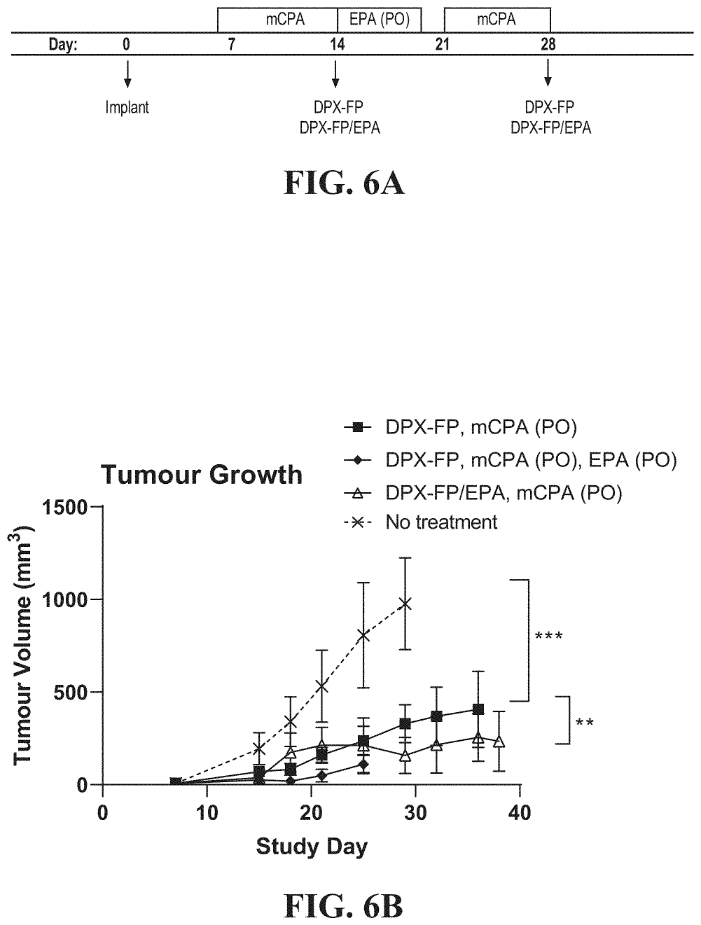

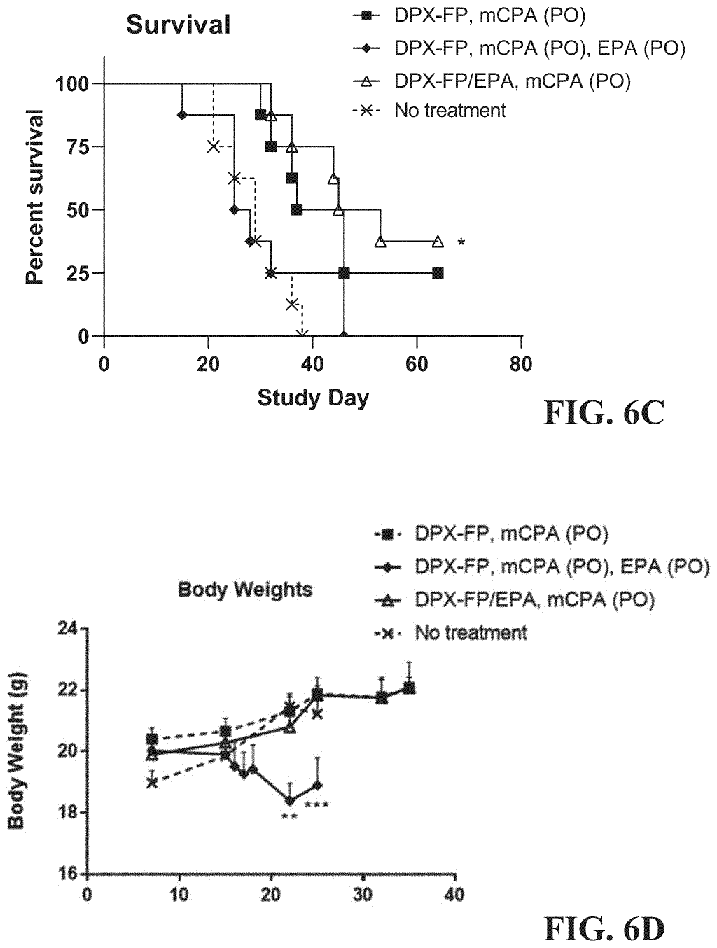

Mice (n=8 per group) were implanted with C3 cancer cells on study day 0. Mice in all treatment groups were treated with 0.4 mg metronomic cyclophosphamide (mCPA) daily for seven days through drinking water starting on study day 7 and 21 (oral administration: PO). One group was untreated as a control. One treatment group was treated with epacadostat (EPA) through oral gavage 6 mg/day for five days starting on study day 14 for a total EPA dose of 30 mg over 5 days. Mice were treated with a composition of the invention (DPX) containing either FP alone (DPX-FP) or FP in combination with EPA (DPX-FP/EPA, 1 mg EPA). DPX was administered by subcutaneous injection to the treatment groups on study day 14 and 28, for a total EPA dose of 2mg over 14 days in the group receiving DPX-FP/EPA. The DPX compositions were prepared according to the methods detailed in Example 1. All DPX-FP and DPX-FP/EPA formulations contained 20 micrograms of DNA based PolyI:C polynucleotide adjuvant. The freeze-dried vials were reconstituted in 0.7 mL of Montanide® ISA 51 VG (SEPPIC, France) to obtain a clear solution (FIG. 1; Panel D). The schedule of experimental treatment is shown in FIG. 6A.

FIG. 6B shows that mice treated with DPX-FP/EPA exhibited significantly lower tumour growth than mice treated with DPX-FP without EPA. As well, mice treated with DPX-FP/EPA exhibited a similar reduction of tumour growth compared to mice treated with DPX-FP and oral EPA (PO). This demonstrates that delivery of EPA in a composition of the invention effectively restricted tumour growth using a lower dose than conventional oral delivery of EPA.

FIG. 6C shows that mice treated with DPX-FP/EPA exhibited a significantly higher survival rate than mice treated with DPX-FP and oral EPA (PO). FIG. 6D shows that mice treated with DPX-FP and oral EPA (PO) exhibited significant weight loss during treatment, demonstrating the toxicity of EPA treatment. FIG. 6D also shows that mice treated with DPX-FP/EPA did not exhibit weight loss during treatment, similar to mice treated with DPX-FP without EPA. Together, this demonstrates that delivery of EPA in a composition of the invention reduced the toxicity of EPA treatment and improved survival.

### Example 3

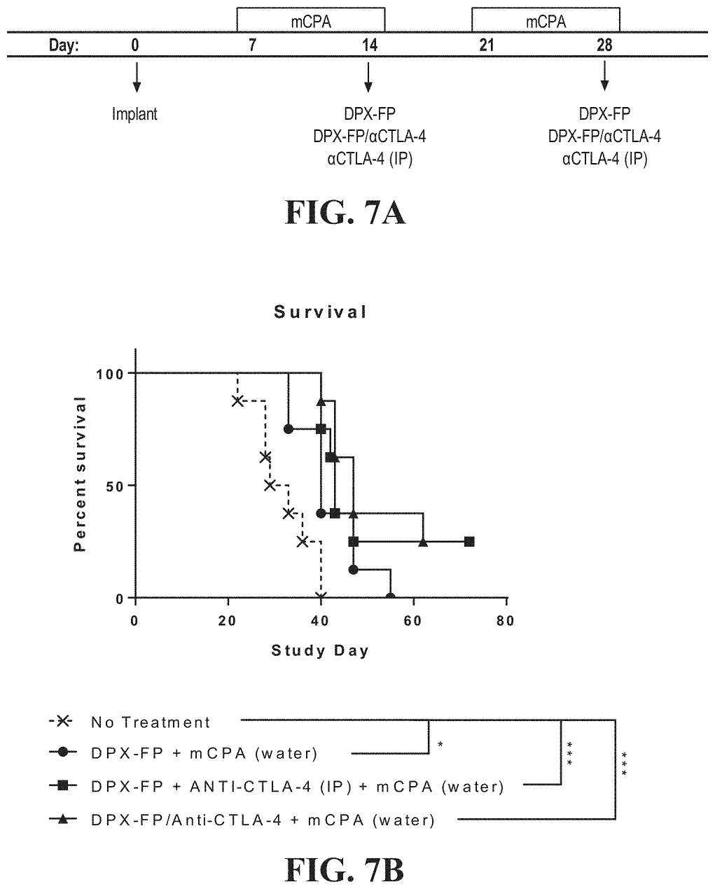

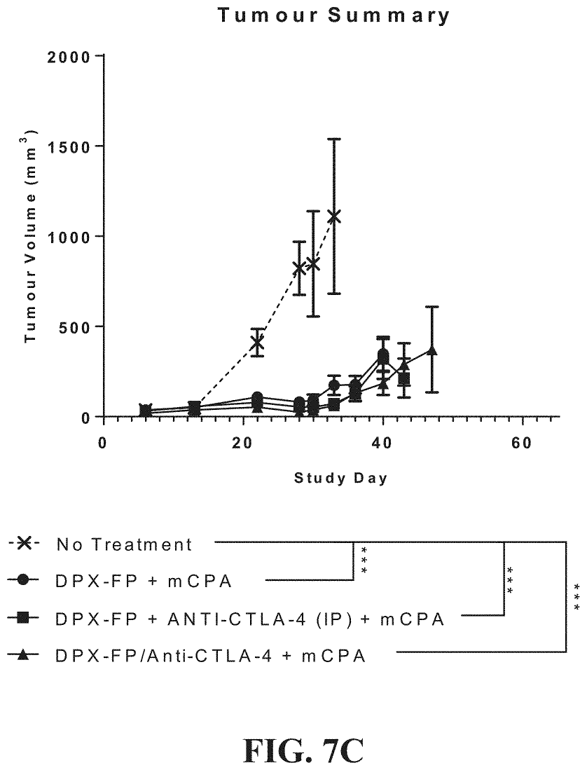

Mice (n=8 per group) were implanted with C3 cancer cells on study day 0 and treated with metronomic cyclophosphamide (mCPA) at 20 mg/kg/day for seven days starting on study day 7 and 21 (oral administration: PO). One group was untreated as a control. The treatment groups received a subcutaneous injection of a composition of the invention (DPX) containing either FP alone (DPX-FP), containing FP and Anti-CTLA-4 (DPX-FP/Anti-CTLA-4, 0.1mg Anti-CTLA-4), or containing FP alone and provided alongside an intra-peritoneal (IP) injection of 0.1 mg Anti-CTLA-4. Both DPX-FP and DPX-FP/Anti-CTLA-4 formulations contained 20 micrograms of DNA based PolyI:C polynucleotide adjuvant. DPX-FP composition was prepared according to the methods detailed in Example 1. For DPX-FP/Anti-CTLA-4 preparation, a 10:1 (w:w) homogenous mixture of DOPC and cholesterol (Lipoid GmbH, Germany) was added to sodium acetate, 50 mM, pH 7.42 at a concentration of 132 mg/ml, with shaking at 300 RPM for about 1 hour to form lipid nanoparticles. The mixture was then extruded by passing the material 25 times through a 200 nm polycarbonate membrane and 10 times through a 100 nm polycarbonate membrane to attain a mean particle size of <120 nm with a pdi of ≤0.1. In two 3-mL vials, added 400 μL 0.1 M sodium acetate pH 6.0, 212.2 μL of the Anti-CTLA-4 solution (7.54 mg/mL), 800 μL of the prepared sized DOPC/Cholesterol lipid nanoparticles. Vortexed gently to mix. To this mixture, added 16 μL of FP (10 mg/mL), 32 μL of DNA based Poly I:C (10 mg/mL) and 139.8 μL sterile water, mixed gently by vortexing. The vials were freeze-dried and then reconstituted with 0.7 mL of Montanide® ISA 51 VG (SEPPIC, France) to obtain a clear solution (FIG. 1; Panel E). Injections in all treatment groups were given on study day 14 and 28. The schedule of experimental treatment is shown in FIG. 7A.

FIGS. 7B and 7C show the unexpected results that mice treated with DPX-FP/Anti-CTLA-4 exhibited a significantly improved survival rate and a significant reduction in tumour growth, compared to untreated mice, equivalent to the survival rate and reduction in tumour growth of mice treated with systemic Anti-CTLA-4 by IP injection. This demonstrates that Anti-CTLA-4 delivered in a composition of the invention effectively restricted tumour growth and improved survival equivalently to administration of Anti-CTLA-4 through systemic delivery by IP injection.

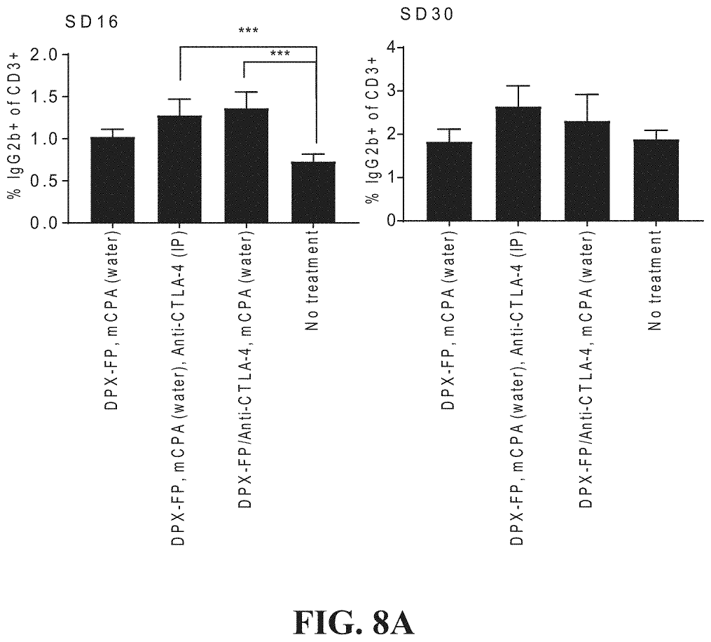

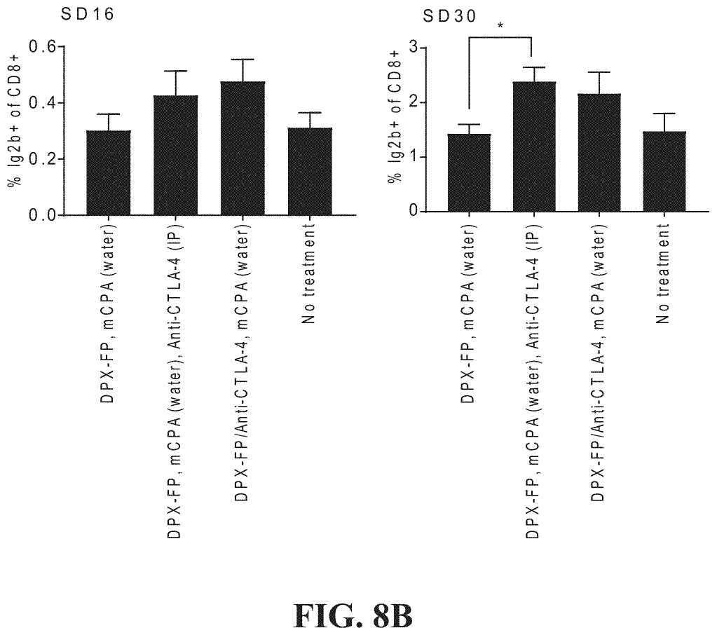

Blood samples were collected from mice from all groups on study day 16 and 30 to assess for Anti-CTLA-4 (IgG2b) bound to circulating T cells. FIGS. 8A and 8B show the unexpected results that mice treated with DPX-FP/Anti-CTLA-4 had similar numbers of circulating CD3+ and CD8+ T cells bound by Anti-CTLA-4 (IgG2b) compared to mice treated with systemic Anti-CTLA-4 by IP injection. This demonstrates that Anti-CTLA-4 delivered in a composition of the invention effectively bound to circulating T cells equivalently to administration of Anti-CTLA-4 through a systemic delivery route such as IP injection.

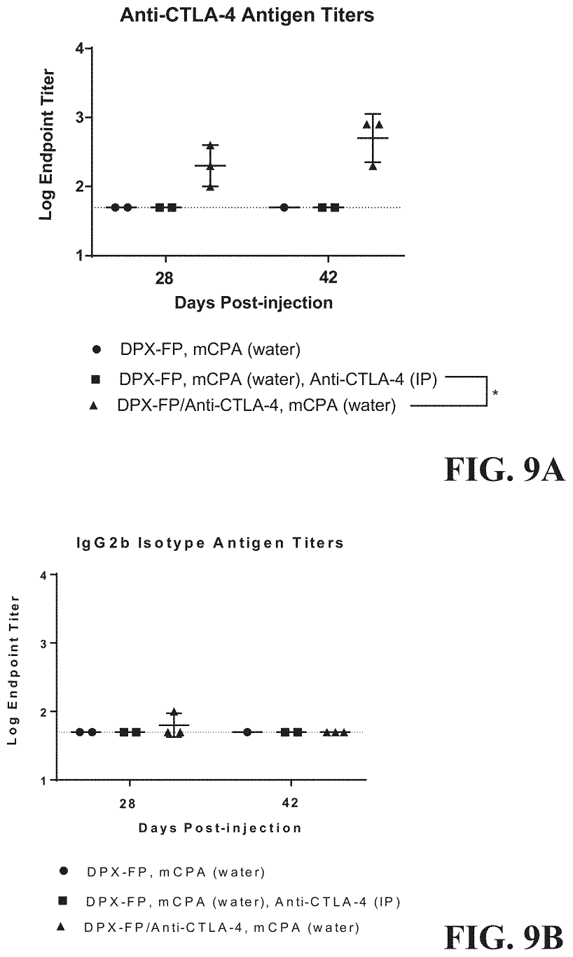

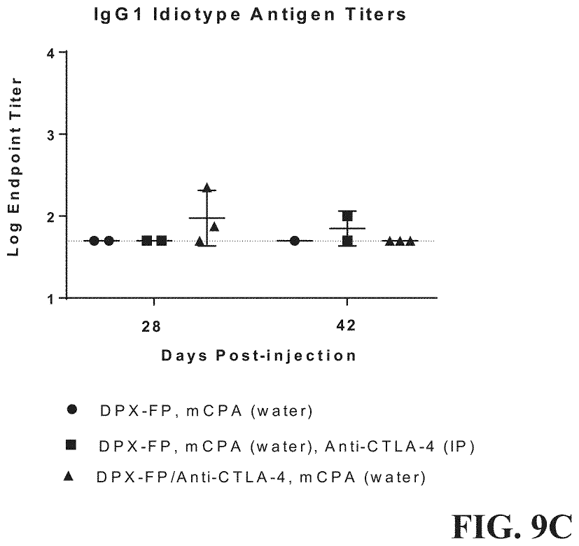

Serum samples were collected at 28 and 42 days post-injection from mice in all groups and were assessed for anti-drug antibody (ADA) formation by bridging ELISA. For the bridging ELISA, briefly, the coating antigen was either anti-CTLA-4 which is a mouse IgG2b, or an IgG2b isotype control or an IgG1 isotype control and the detection antibody was anti-CTLA-4. FIG. 9A shows that mice treated with DPX-FP/Anti-CTLA-4 developed ADA against anti-CTLA-4. This demonstrates that, unexpectedly, the formation of ADA does not impede the effect of Anti-CTLA-4 delivered in a composition of the invention in improving survival (FIG. 7B), restricting tumour growth (FIG. 7C), and binding to circulating T cells (FIGS. 8A and 8B).

### Example 4

C57B1/6 mice (n=3 per time point) were injected subcutaneously on study day 0 with 1 mg of Evans Blue (EVB) dye formulated either in a composition of the invention (DPX, group 1) or in an aqueous solution (group 2). Control groups were injected subcutaneously with DPX not containing EVB (group 3) or not given an injection (group 4). DPX compositions were prepared according to the methods detailed in Example 1. The freeze-dried vial was reconstituted in Montanide® ISA 51 VG (SEPPIC, France). Cells were collected from the vaccine-draining lymph nodes (LN), blood, liver, and spleen from 2-3 mice of each group on study days 1, 2, 5, and 7, and the cells were stained to detect different cell populations by flow cytometry.

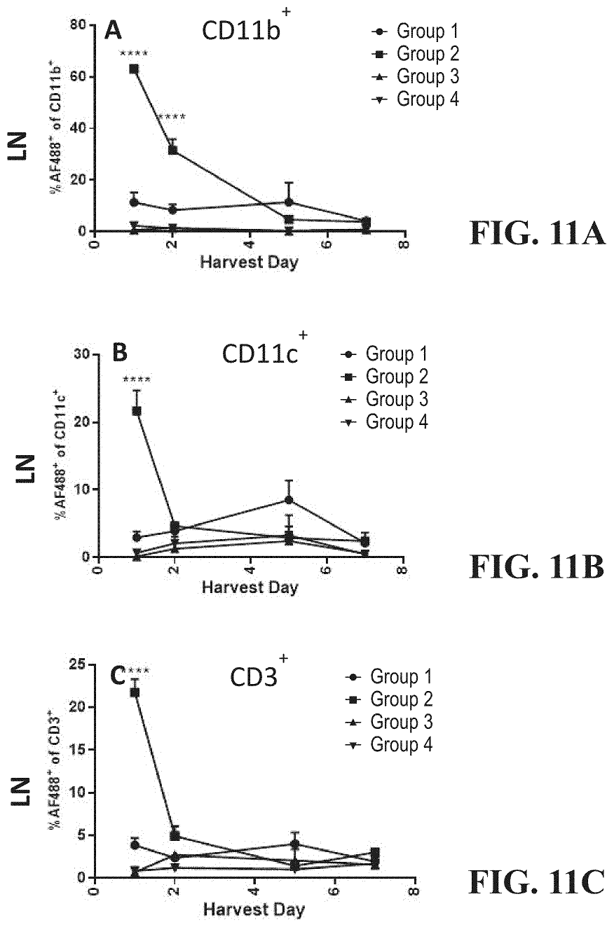

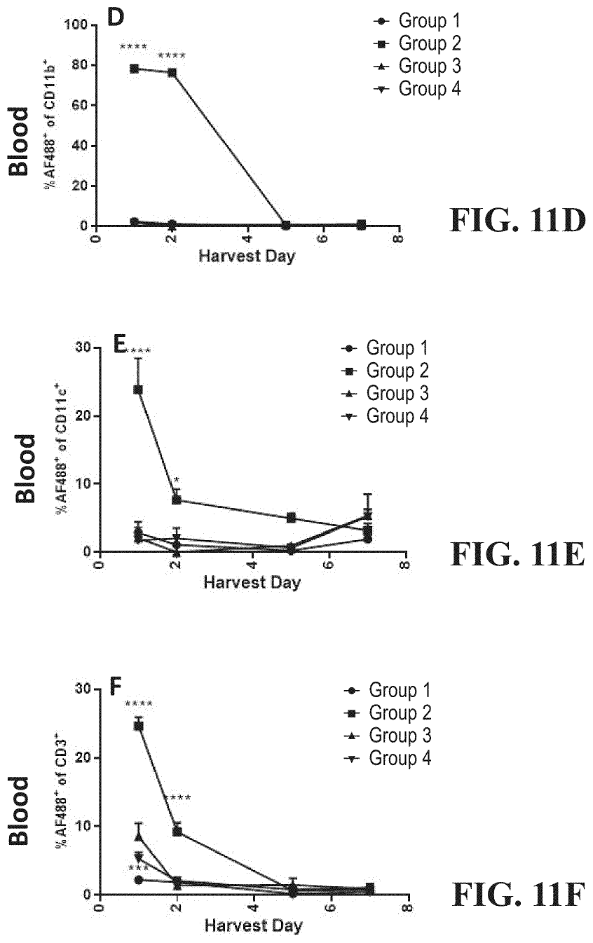

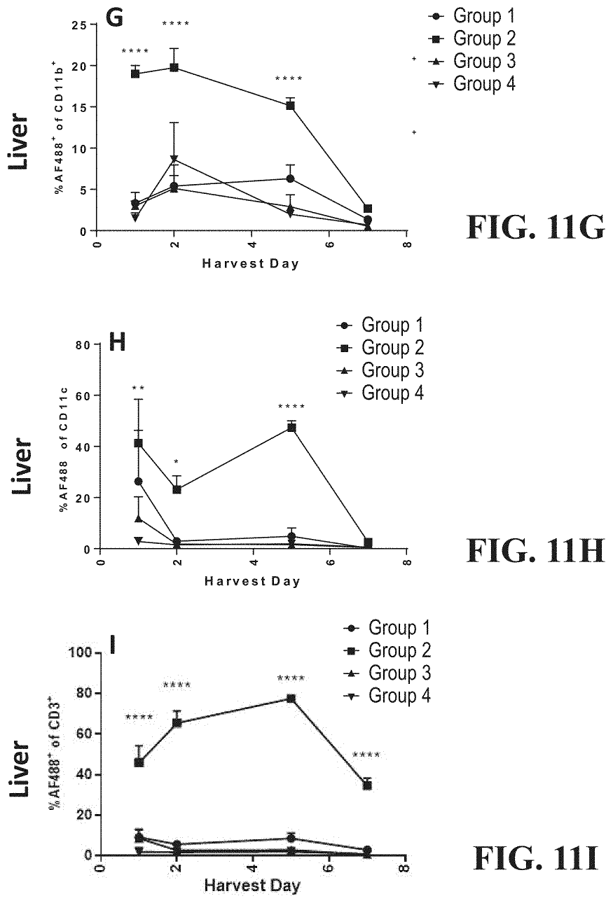

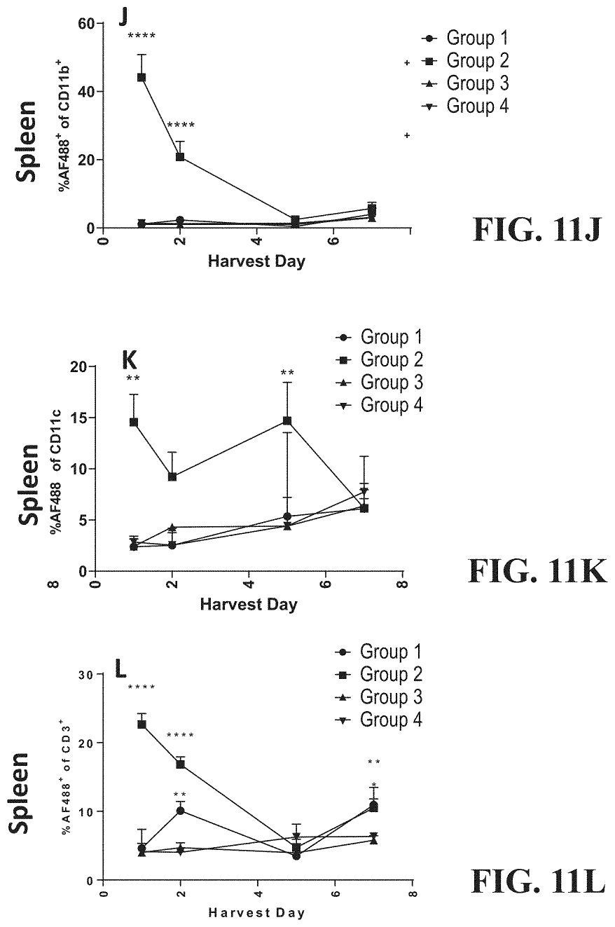

EVB (MW 960.81 g/mol) is a membrane impermeant dye with high affinity for serum albumin that can penetrate into non-viable cells or be actively internalized by phagocytic cells, such as macrophages and dendritic cells. FIG. 11 shows the percentage of different cell types (CD11b+ macrophages; CD11c+ dendritic cells; CD3+ T cells) from different tissues (LN; Blood; Liver; Spleen) and at different time points (days 1, 2, 5, and 7) that contained EVB.

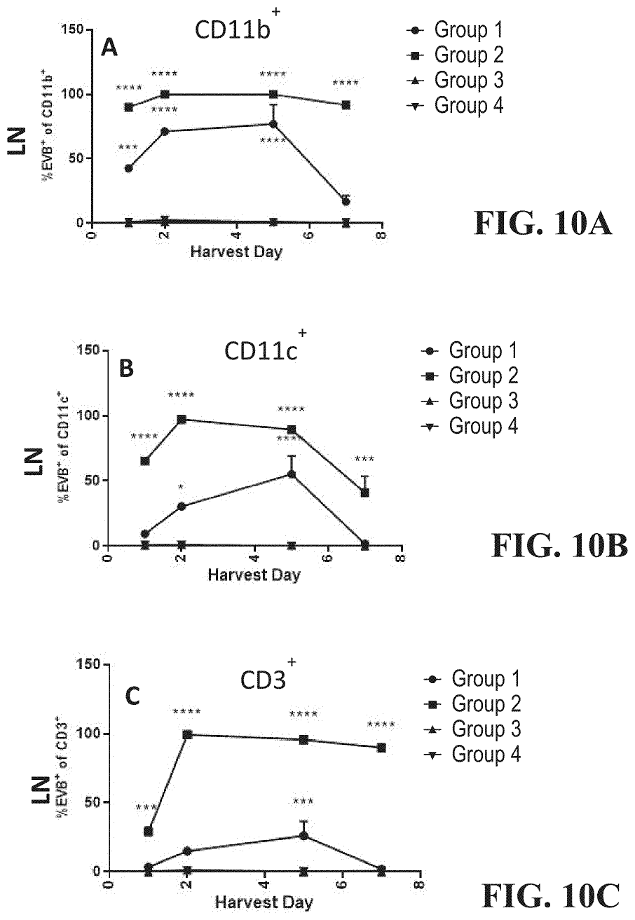

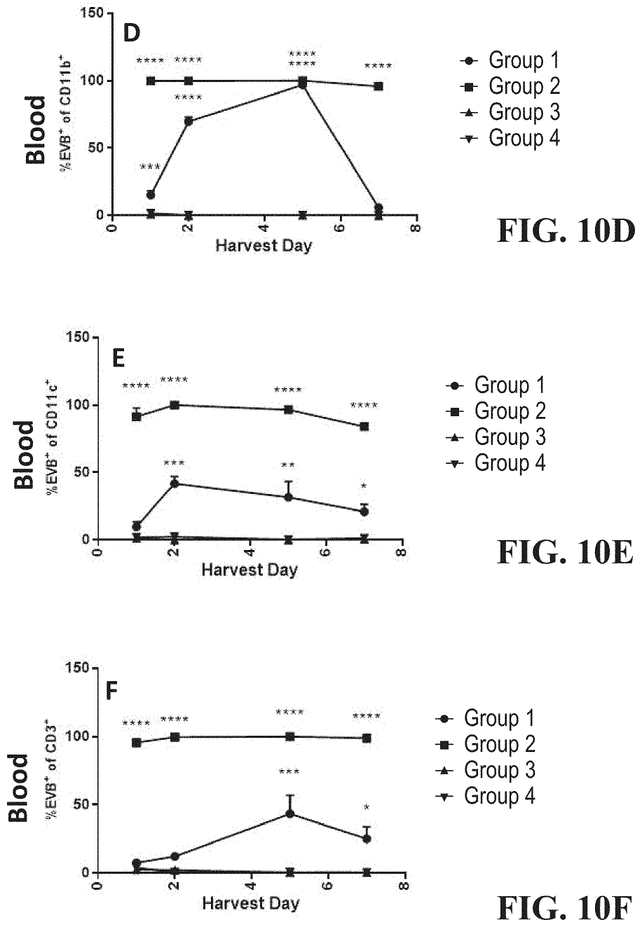

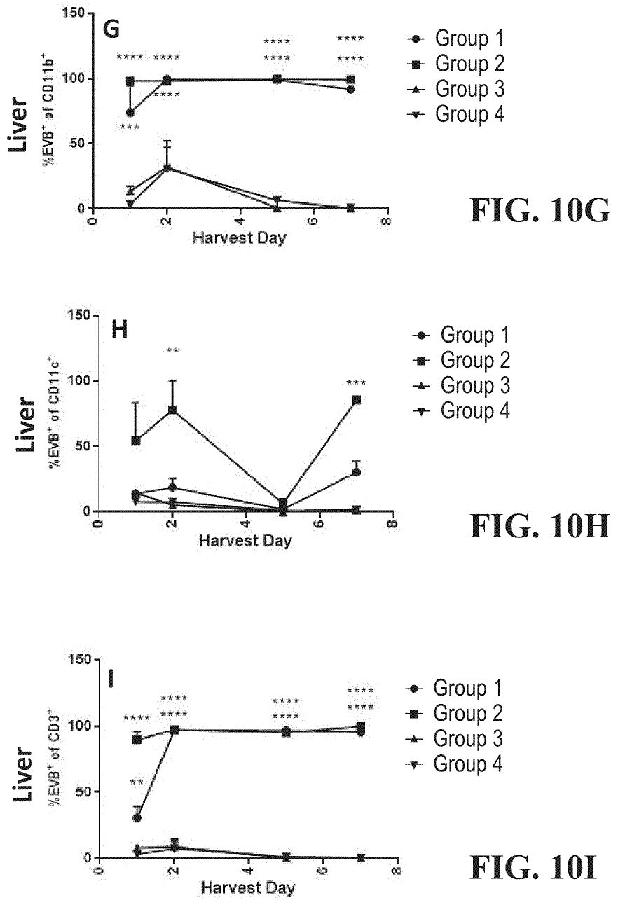

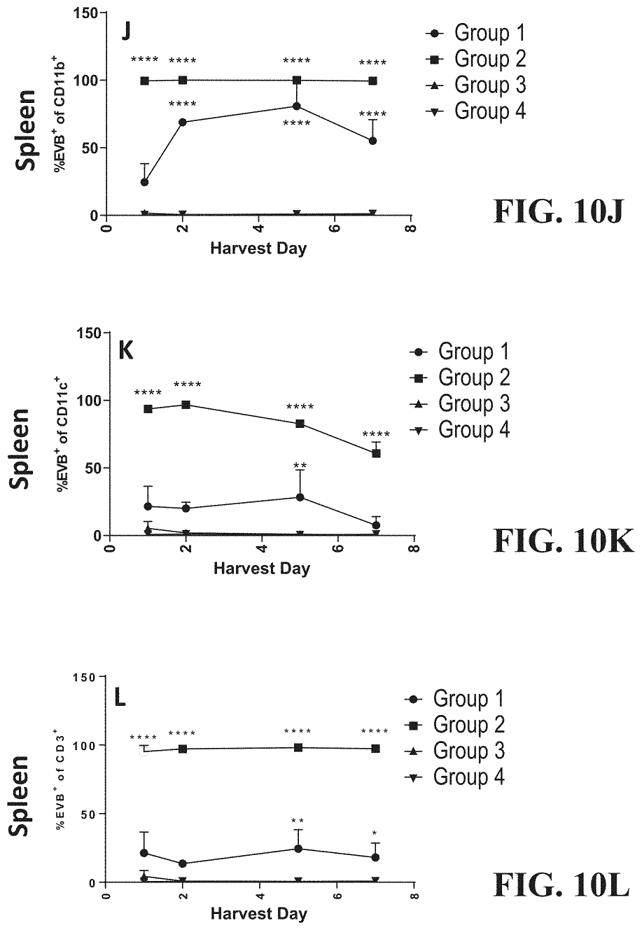

FIG. 10A shows that in the LN group 1 had a significant number of EVB⁺ CD11b⁺ cells on days 1, 2 and 5 compared to group 3, while group 2 had a significant number of EVB⁺ CD11b⁺ cells on all days tested compared to group 3. FIG. 10B shows that in the LN group 1 had a significant number of EVB⁺ dendritic cells on days 2 and 5 compared to group 3, while group 2 had a significant number of EVB⁺ dendritic cells on all days tested compared to group 3. FIG. 10C shows that in the LN group 1 had a significant number of EVB⁺ T cells on day 5 compared to group 3, while group 2 had a significant number of EVB⁺ T cells on all days tested compared to group 3. FIG. 10D shows that in the blood group 1 had a significant number of EVB⁺ CD11b⁺ cells on days 1, 2 and 5 compared to group 3, while group 2 had a significant number of EVB⁺ CD11b⁺ cells on all days tested compared to group 3. FIG. 10E shows that in the blood group 1 had a significant number of EVB⁺ dendritic cells on days 2, 5, and 7 compared to group 3, while group 2 had a significant number of EVB⁺dendritic cells on all days tested compared to group 3. FIG. 1OF shows that in the blood group 1 had a significant number of EVB⁺ T cells on day 5 and 7 compared to group 3, while group 2 had a significant number of EVB⁺ T cells on all days tested compared to group 3. FIG. 10G shows that in the liver both group 1 and group 2 had a significant number of EVB⁺ CD11b⁺ cells on all days tested compared to group 3. FIG. 10H shows that in the liver there was no significance between group 1 and 3 on any day, while group 2 had a significant number of EVB⁺ dendritic cells on days 2 and 7 tested compared to group 3. FIG. 10I shows that in the liver both group 1 and group 2 had a significant number of EVB⁺ T cells on all days tested compared to group 3. FIG. 10J shows that in the spleen group 1 had a significant number of EVB⁺ CD11b⁺ cells on days 2, 5 and 7 compared to group 3, while group 2 had a significant number of EVB⁺ CD11b⁺ cells on all days tested compared to group 3. FIG. 10K shows that in the spleen group 1 had a significant number of EVB⁺dendritic cells on day 5 compared to group 3, while group 2 had a significant number of EVB⁺dendritic cells on all days tested compared to group 3. FIG. 10L shows that in the spleen group 1 had a significant number of EVB⁺ T cells on days 5 and 7 compared to group 3, while group 2 had a significant number of EVB⁺ T cells on all days tested compared to group 3.

FIGS. 10A-C show that in mice injected with EVB in aqueous solution (group 2) cells in the LN were quickly saturated (˜100% of cells EVB+ by day 2), including CD3+ T cells that are not phagocytic. EVB had still not been cleared from the LN by day 7 in group 2. In mice injected with EVB in DPX (group 1), EVB exhibited a sustained release into the LN that peaked at day 5, was largely cleared by day 7, that did not saturate cells in the LN, and was preferentially taken by CD11b+ macrophages and CD11c+ dendritic cells rather than CD3+ T cells. This demonstrates that EVB delivered in a composition of the invention exhibited a sustained release, preferentially in phagocytic cells, to a draining LN compared to EVB delivered in an aqueous solution.

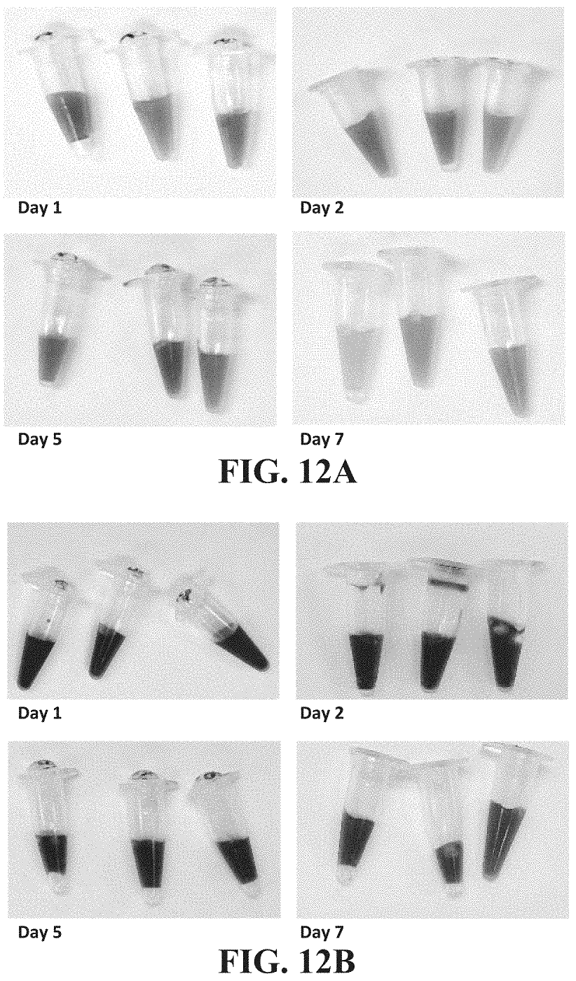

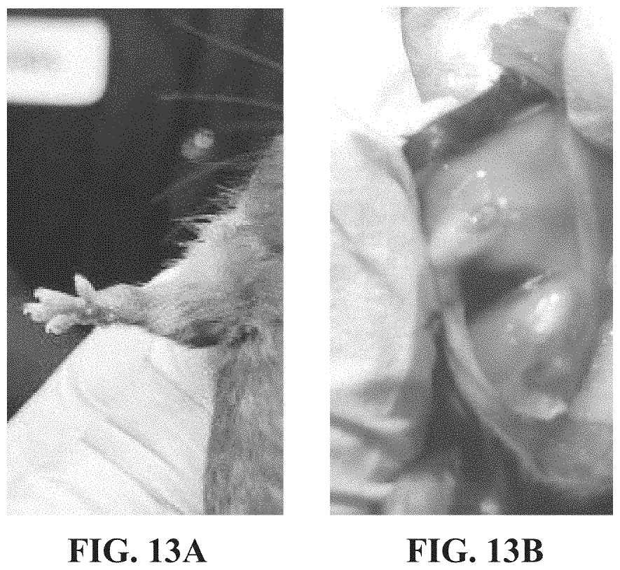

FIGS. 10D-F show that in mice injected with EVB in aqueous solution (group 2) cells in the blood were quickly saturated (˜100% of cells EVB+ by day 2), including CD3+ T cells that are not phagocytic. EVB has still not been cleared from the blood by day 7 in group 2, including in CD11b+ macrophages and CD3+ T cells that were still nearly saturated on day 7. In mice injected with EVB in DPX (group 1), EVB exhibited a lower release into the blood compared to group 2. Group 1 mice did not exhibit saturation of CD11c+ dendritic cells or CD3+ T cells in the blood, and the saturation of CD11b+ macrophages by day 5 was completely cleared by day 7. This demonstrates that EVB delivered in the composition of the invention exhibited a limited systemic release into the blood compared to EVB delivered in an aqueous solution that quickly saturated circulating cells in the blood. FIG. 12A shows that plasma from group 1 mice was light red in colour whereas FIG. 12B shows that plasma from group 2 mice was dark blue, demonstrating that EVB delivered in a composition of the invention was limited in entering circulation compared to EVB delivered in an aqueous solution. FIG. 13A shows that the skin of group 2 mice turned blue whereas FIGS. 13B and 13C show that blue colouration was sequestered at the site of injection and in the draining LN in group 1 mice, demonstrating that EVB delivered in a composition of the invention was preferentially targeted to the draining LN and sequestered from systemic release.

FIGS. 10G-I show that in mice injected with EVB in aqueous solution (group 2) macrophages and T cells in the liver were quickly saturated (˜100% of cells EVB+ by day 1-2) and a large number dendritic cells were EVB+ by day 2. In mice injected with EVB in DPX (group 1), EVB exhibited a slower release into the liver (no saturation on day 1) and there were significantly fewer EVB+CD11c+ dendritic cells in the liver compared to group 2. This demonstrates that EVB delivered in the composition of the invention exhibited a more limited release into the liver compared to EVB delivered in an aqueous solution.

FIGS. 10J-L show that in mice injected with EVB in aqueous solution (group 2) cells in the spleen were quickly saturated (˜100% of cells EVB+ by day 1-2), including CD3+ T cells that are not phagocytic, and was still saturated in CD11b+ macrophages and T cells by day 7. In mice injected with EVB in DPX (group 1), EVB exhibited a sustained release into the spleen that peaked at day 5, began to be cleared by day 7, that did not saturate cells in the spleen, and was preferentially taken by CD11b+ macrophages rather than CD3+ T cells. This demonstrates that EVB delivered in a composition of the invention exhibited a sustained release, preferentially in phagocytic cells, into the spleen to the spleen compared to EVB delivered in an aqueous solution.

### Example 5

C57B1/6 mice (n=2-3 per time point) were injected subcutaneously on study day 0 with 0.1 mg of AlexaFluor488 hydrazide (AF488) dye formulated either in a composition of the invention (DPX, group 1) or in an aqueous solution (group 2). Control groups were injected subcutaneously with DPX not containing AF488 (group 3) or not given an injection (group 4). DPX compositions were prepared according to the methods detailed in Example 1. The freeze-dried vial was reconstituted in Montanide® ISA 51 VG (SEPPIC, France). Cells were collected from the vaccine-draining lymph nodes (LN), blood, liver, and spleen from 2-3 mice of each group on study days 1, 2, 5, and 7, and the cells were stained to detect different cell populations by flow cytometry.

AF488 (MW 773.91 g/mol) is a membrane impermeant fluorescent dye that can be actively internalized by phagocytic cells, such as macrophages and dendritic cells. FIG. 11 shows the percentage of different cell types (CD11b+ macrophages; CD11c+ dendritic cells; CD3+ T cells) from different tissues (LN; Blood; Liver; Spleen) and at different time points (days 1, 2, 5, and 7) that contained AF488.

FIG. 11A shows that in the LN there was no significance between group 1 and 3 on any day, while group 2 had a significant number of AF488⁺CD11b⁺ cells on days 1 and 2 compared to group 3. FIG. 11B shows that in the LN there was no significance between group 1 and 3 on any day, while group 2 had a significant number of AF488⁺ dendritic cells on day 1 compared to group 3. FIG. 11C shows that in the LN Group 1 was not significantly different compared to group 3 on any day, while group 2 had a significant number of AF488⁺ T cells on day 1 compared to group 3. FIG. 11D shows that in the blood there was no significance between group 1 and 3 on any day, while group 2 had a significant number of AF488⁺ CD11b⁺ cells on days 1 and 2 compared to group 3. FIG. 11E shows that in the blood there was no significance between group 1 and 3 on any day, while group 2 had a significant number of AF488⁺ dendritic cells on days 1 and 2 compared to group 3. FIG. 11F shows that in the blood group 1 was not significantly different compared to group 3 on any day, while group 2 had a significant number of AF488⁺ T cells on days 1 and 2 compared to group 3. FIG. 11G shows that in the liver there was no significance between group 1 and 3 on any day, while group 2 had a significant number of AF488⁺ monocytes on days 1, 2, and 5 compared to group 3. FIG. 11H shows that in the liver there was no significance between group 1 and 3 on any day, while group 2 had a significant number of AF488⁺ dendritic cells on days 1, 2, and 5 compared to group 3. FIG. 11I shows that in the liver group 1 was not significantly different compared to group 3 on any day, while group 2 had a significant number of AF488⁺ T cells on all days collected compared to group 3.

FIGS. 11A-C show that in mice injected with AF488 in aqueous solution (group 2) AF488+ cells in the LN quickly peaked at day 1 and AF488 was cleared by day 5-7. In mice injected with AF488 in DPX (group 1), AF488 exhibited a sustained release into the LN that peaked at day 5, preferentially in CD11b+ and CD11c+ cells rather than in CD3+ T cells. This demonstrates that AF488 delivered in a composition of the invention exhibited a sustained release, preferentially in phagocytic cells, to a draining LN compared to AF488 delivered in an aqueous solution.

FIGS. 11D-F show that in mice injected with AF488 in aqueous solution (group 2) AF488+ cells in the blood quickly peaked at day 1 with over 70% of circulating CD11b+cells being AF488+ on days 1-2. In mice injected with AF488 in DPX (group 1), AF488 did not exhibit release into the blood, with AF488+ cells in the blood being comparable to that in the control groups. This demonstrates that AF488 delivered in the composition of the invention was not released into cells in the blood compared to AF488 delivered in an aqueous solution that exhibited a quick and significant release of AF488+ cells into the blood.

FIGS. 11G-I show that in mice injected with AF488 in aqueous solution (group 2) there was a rapid and significant release of AF488 into the spleen in all cell types. In mice injected with AF488 in DPX (group 1), there was very little release of AF488 into the spleen comparable to the control groups.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used throughout herein, the term “about” means reasonably close. For example, “about” can mean within an acceptable standard deviation and/or an acceptable error range for the particular value as determined by one of ordinary skill in the art, which will depend on how the particular value is measured. Further, when whole numbers are represented, about can refer to decimal values on either side of the whole number. When used in the context of a range, the term “about” encompasses all of the exemplary values between the one particular value at one end of the range and the other particular value at the other end of the range, as well as reasonably close values beyond each end.

As used herein, whether in the specification or the appended claims, the transitional terms “comprising”, “including”, “carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the invention disclosed and/or claimed herein.

### REFERENCES

1) Bertschinger, J.; Grabulovski, D.; and Neri, D. (2007) “Selection of single domain binding proteins by covalent DNA display”, Protein Eng Des Sel 20(2):57-68.
2) Berge, S. M.; Bighley, L. D.; and Monkhouse, D. C. (1977) “Pharmaceutical salts”, J Pharm Sci 66(1):1-19.
3) Beste, G.; Schmidt, F. S.; Stibora, T.; and Skerra, A. (1999) “Small antibody-like proteins with prescribed ligand specificities derived from the lipocalin fold”, Proc Natl Acad Sci U S A. 96(5):1898-1903.
4) Boschek, C. B.; Apiyo, D. O.; Soares, T. A.; Engelmann, H. E.; Pefaur, N. B.; Straatsma, T. P.; and Baird, C. L. (2009) “Engineering an ultra-stable affinity reagent based on Top7”, Protein Engineering, Design and Selection 22(5):325-332.
5) Curiel, T. J.; Coukos, G.; Zou, L.; Alvarez, X.; Cheng, P.; Mottram, P.; Evdemon-Hogan, M.; Conejo-Garcia, J. R.; Zhang, L.; Burow, M.; Zhu, Y.; Wei, S.; Kryczek, I.; Daniel, B.; Gordon, A.; Myers, L.; Lackner, A.; Disis, M.L.; Knutson, K. L.; Chen, L.; and Zou, W. (2004a) “Specific recruitment of regulatory T cells in ovarian carcinoma fosters immune privilege and predicts reduced survival”, Nat Med 10(9):942-949.
6) Curiel, T. J.; Morris, C.; Brumlik, M.; Landry, S. J.; Finstad, K.; Nelson, A.; Joshi, V.;

Hawkin, C.; Alarez, X.; Lackner, A.; and Mohamadzadeh, M. (2004b) “Peptides identified through phage display direct immunogenic antigen to dendritic cells”, J Immunol 172:7425-7431.
7) Delcroix, M; and Riley, L. W. (2010) “Cell-Penetrating Peptides for Antiviral Drug Development”, Pharmaceuticals (Basel) 3(3):448-470.
8) Desmet, J.; Verstraete, K.; Bloch, Y.; Lorent, E.; Wen, Y.; Devreese, B.; Vandenbroucke, K.; Loverix, S.; Hettmann, T.; Deroo, S.; Somers, K.; Henderikx, P.; Lasters, I.; and Savvides, S. N. (2014). “Structural basis of IL-23 antagonism by an Alphabody protein scaffold”, Nature Communications 5:5237.
9) Feldwisch, J.; and Tolmachev, V. (2012) “Engineering of affibody molecules for therapy and diagnostics”, Methods Mol Biol 899:103-26.
10) Gebauer, M.; and Skerra, A. (2009) “Engineered protein scaffolds as next-generation antibody therapeutics”, Curr Opinion in Chemical Biology 13:245-255.
11) Grabulovski, D.; Kaspar, M.; and Neri, D. (2007) “A Novel, Non-immunogenic Fyn SH3-derived Binding Protein with Tumor Vascular Targeting Properties”, J Biol Chem 282:3196-3204.
12) Green, L. L.; Hardy, M. C.; Maynard-Currie, C. E.; Tsuda, H.; Louie, D. M.; Mendez, M. J.; Abderrahim, H.; Noguchi, M.; Smith, D. H.; Zeng, Y.; David, N. E.; Sasai, H.; Garza, D.; Brenner, D. G.; Hales, J. F.; McGuinness, R. P.; Capon, D. J.; Klapholz, S.; Jakobovits, A. (1994) “Antigen-specific human monoclonal antibodies from mice engineered with human Ig heavy and light chain YACs”, Nature Genet 7:13-21.
13) Greenfield, E. A. (ed.) (2014) “Antibodies: A Laboratory Manual”, Cold Spring Harbor Laboratory Press, 2^{nd} Edition.
14) Hollinger, P.; and Hudson, P. J. (2005) “Engineered antibody fragments and the rise of single domains”, Nat Biotechnol 23(9):1126-1136.
15) Johnson, K. S.; and Chiswell, D. J. (1993) “Human antibody engineering”, Current Opinion in Structural Biology 3:564-571.
16) Lonberg, N.; Taylor, L. D.; Harding, F. A.; Trounstine, M.; Higgins, K. M.; Schramm, S. R., Kuo, C.-C.; Mashayekh, R.; Wymore, K.; McCabe, J. G.; O'Regan, D. M.; O'Donnell, S.L.; Lapachet, E. S. G.; Bengoechea, T.; Fishwild, D. M.; Carmack, C.E.; Kay, R. M.; and Huszar, D. (1994) “Antigen-specific human antibodies from mice comprising four distinct genetic modifications”, Nature 368:856-859.
17) McCafferty, J.; Griffiths, A. D.; Winter, G.; and Chiswell, D. J. (1990) “Phage antibodies: filamentous phage displaying antibody variable domains”, Nature 348:552-553.
18) Mouratou, B.; Behar, G.; Paillard-Laurance, L.; Colinet, S.; and Pecorari, F. (2012) “Ribosome display for the selection of Sac7d scaffolds”, Methods Mol Biol 805:315-31.
19) Nagaraj, S.; and Gabrilovich, D. I. (2008) “Tumor escape mechanism governed by myeloid-derived suppressor cells”, Cancer Res 68:2561-2563.
20) Pardoll, D. M. (2012) “The blockade of immune checkpoints in cancer immunotherapy”, Nature Reviews Cancer 12:252-264.
21) Schlatter, D.; Brack, S.; Banner, D. W.; Batey, S.; Benz, J.; Bertschinger, J.; Huber, W.; Joseph, C.; Rufer, A. C.; van der Klooster, A.; Weber, M.; Grabulovski, D.; and Hennig, D. (2012) “Generation, characterization and structural data of chymase binding proteins based on the human Fyn kinase SH3 domain”, MAbs 4(4):497-508.
22) Suderman, R. J., Rice, D. A., Gibson, S. D., Strick, E. J., Chao, D. M. (2017) “Development of polyol-responsive antibody mimetics for single-step protein purification”, Protein Expression and Purification 134:114-124.
23) Taylor, L. D.; Carmack, C. E.; Huszar, D.; Higgins, K. M.; Mashayekh, R.; Sequar, G.; Schramm, S. R.; Kuo, C. C.; O'Donnell, S. L.; Kay, R. M.; et al. (1994) “Human immunoglobulin transgenes undergo rearrangement, somatic mutation and class switching in mice that lack endogenous IgM”, Int. Immun. 6:579-591.
24) van der Neut Kolfschoten, M.; Schuurman, J.; Losen. M.; Bleeker, W. K.; Martinez-Martinez, P.; Vermeulen, E.; den Bleker, T. H.; Wiegman, L.; Vink, T.; Aarden, L. A.; De Baets, M. H.; van de Winkel, J. G.; Aalberse, R. C.; Parren, P. W. (2007) “Anti-inflammatory activity of human IgG4 antibodies by dynamic Fab arm exchange”, Science 317(5844):1554-1557.
25) Weidle, U. H.; Auer, J.; Brinkmann, U.; Georges, G.; Tiefenthaler, G. (2013) “The emerging role of new protein scaffold-based agents for treatment of cancer” Cancer Genomics Proteomics 10(4): 155-68.
26) Wishart, D. S.; Feunang, Y. D.; Guo, A. C.; Lo, E. J.; Marcu, A.; Grant, J. R.; Sajed, T.; Johnson, D.; Li, C.; Sayeeda, Z.; Assempour, N.; Iynkkaran, I.; Liu, Y.; Maciejewski, A.; Gale, N.; Wilson, A.; Chin, L.; Cummings, R.; Le, D.; Pon, A.; Knox, C.; and Wilson, M. (2017) DrugBank 5.0: a major update to the DrugBank database for 2018, Nucleic Acids Research 46(D1): D1074-D1082.
27) Yang, L.; Pang, Y.; and Moses, H. L. (2010) “TGF-β and immune cells: an important regulatory axis in the tumor microenvironment and progression”, Trends Immunol 31(6): 220-227.
28) Zhang, D.; Wang, J.; and Xu, D. (2016) “Cell-penetrating peptides as noninvasive transmembrane vectors for the development of novel multifunctional drug-delivery systems”, J. Control Release 229:130-139.
29) Yong, X.; Xiao, Y-F.; Luo, G.; He, B.; Lü, M-H.; Hu, C-J.; Guo, H.; and Yang, S-M. (2012) “Strategies for Enhancing Vaccine-Induced CTL Antitumor Immune Responses”, Biomed Biotechnol 2012:605045.

30) Zahid, M.; and Robbins, P. D. (2012) “Protein transduction domains: applications for molecular medicine”, Curr Gene Ther 12:374-380.

1. A method for targeted delivery of an active agent to lymph nodes or lymphoid cells in a lymphatic tissue, said method comprising administering to a subject in need thereof a composition comprising:
a) i) an active agent, wherein the active agent is a small molecule drug; an antibody, an antibody mimetic, or a functional equivalent or functional fragment of any one thereof; or a mixture thereof, or
ii) an immunomodulatory agent
b) one or more lipid-based structures; and
c) a hydrophobic carrier.

2. The method of claim 1, wherein the composition is water-free.

3-6. (canceled)

7. The method of claim 1, wherein the active agent or immunomodulatory agent is delivered to the lymph nodes or lymphoid cells in a lymphatic tissue by dendritic cells or macrophages.

8. The method of claim 7, wherein the active agent or immunomodulatory agent is delivered to the dendritic cells or macrophages at or near the site of administration of the composition.

9. The method of claim 1, wherein the active agent does not directly bind a major histocompatibility complex (MHC) class I protein, an MHC class II protein, or both.

10. (canceled)

11. The method of claim 1, wherein the active agent or immunomodulatory agent is delivered intact to the lymph nodes or lymphoid cells.

12-14. (canceled)

15. The method of claim 1, wherein the active agent is a small molecule drug.

16. (canceled)

17. The method of claim 15, wherein the small molecule drug is a cytotoxic agent, an anti-tumor agent, a chemotherapeutic agent, an anti-neoplastic agent, an antiviral agent, an antibacterial agent, an anti-inflammatory agent, an immunomodulatory agent, an immune response checkpoint agent, a biological response modifier, a prodrug, a cytokine, a chemokine, a vitamin, a steroid, a ligand, an analgesic, a radiopharmaceutical, a radioisotope or a dye for visual detection.

18. The method of claim 15, wherein the active agent is epacadostat, rapamycin, doxorubicin, valproic acid, mitoxantrone, vorinostat, cyclophosphamide, irinotecan, cisplatin, methotrexate, tacrolimus or a pharmaceutically acceptable salt of any one thereof.

19. The method of claim 18, wherein the active agent is epacadostat.

20. The method of claim 18, wherein the active agent is cyclophosphamide.

21. (canceled)

22. The method of claim 1, wherein the active agent is an antibody, an antibody mimetic, or a functional equivalent or functional fragment of any one thereof.

23. The method of claim 22, wherein the active agent is an anti-CTLA-4 antibody.

24. (canceled)

25. The method of claim 22, wherein the active agent is an anti-PD-1 antibody or an anti-PD-L1 antibody.

26. (canceled)

27. The method of claim 22, wherein the active agent is a peptide aptamer, an affimer, an affilin, an affibody, an affitin, an alphabody, an anticalin, an avimer, a DARPin, a fynomer, a Kunitz domain peptide, a monobody, a nanoCLAMP, an affinity reagent, a scaffold protein, a monoclonal antibody, a chimeric antibody, a humanized antibody, a F(ab′)₂, a F(ab)₂, a Fab′, a Fab, a Fab₂, a Fab3, a Fv, a scFv, a Dab, an evibody, a minibody, a diabody, a triabody, a tetrabody, or a single-domain antibody (nanobody).

28. The method of claim 1, wherein the active agent or immunomodulatory agent exhibits systemic delivery in the subject when administered in a composition that does not include one or both of the components (b) and (c).

29-38. (canceled)

39. The method of claim 1, wherein the immunomodulatory agent upregulates, downregulates or reprograms the type of immune response that is activated by an antigen or immunogen.

40-42. (canceled)

43. The method of claim 1, wherein the one or more lipid-based structures have a single layer lipid assembly and comprise aggregates of lipids with the hydrophobic part of the lipids oriented outwards toward the hydrophobic carrier and the hydrophilic part of the lipids aggregating as a core.

44-46. (canceled)

47. The method of claim 1, wherein the hydrophobic carrier is mineral oil or a mannide oleate in mineral oil solution.

48-49. (canceled)

50. The method of claim 1, wherein the administration is by subcutaneous, intramuscular, or intraperitoneal injection.

51-58. (canceled)
